(19) United States
(12) Patent Application Publication
(10) Pub. No.: US 2024/0199728 A1
(43) Pub. Date: Jun. 20, 2024

(54) METHODS OF TREATING OBESITY, DIABETES, AND LIVER DYSFUNCTION

(71) Applicant: REGENERON PHARMACEUTICALS, INC., Tarrytown, NY (US)

(72) Inventors: Sandra KLEINER, Biberach an der Riß (DE); Jason MASTAITIS, Yorktown Heights, NY (US); Mark W. SLEEMAN, New York, NY (US)

(21) Appl. No.: 18/472,160

(22) Filed: Sep. 21, 2023

Related U.S. Application Data
(60) Provisional application No. 63/376,582, filed on Sep. 21, 2022.
(60) Provisional application No. 63/508,458, filed on Jun. 15, 2023.

(51) Int. Cl. C07K 16/22; A61K 38/26; A61K 39/00; A61P 3/04; A61P 3/10; C07K 16/26

(52) U.S. Cl. CPC C07K 16/22; A61K 38/26; A61P 3/04; A61P 3/10; C07K 16/26; A61K 2039/507

(57) ABSTRACT

The present disclosure relates to compositions and methods for improving glucose control, increasing lean body mass, reducing fat mass, treating obesity, diabetes, and/or treating liver dysfunction in a subject. More specifically, the disclosure relates to compositions comprising a GDF-8 inhibitor and a GLP-1 agonist and uses thereof, as well as to compositions comprising a GDF-8 inhibitor, an Activin A inhibitor, and a GLP-1 agonist and uses thereof.

### CROSS-REFERENCE TO RELATED APPLICATIONS

This application claims priority to U.S. Provisional Patent Application Nos. 63/376,582, filed on Sep. 21, 2022, and 63/508,458, filed on Jun. 15, 2023, the entire contents of each of which are incorporated by reference herein.

### REFERENCE TO A SEQUENCE LISTING XML

This application contains a Sequence Listing which has been submitted electronically in XML format. The Sequence Listing XML is incorporated herein by reference. Said XML file, created on Sep. 19, 2023, is named 40848_0107USU1_SL.xml and is 24.7 kilobytes in size.

### FIELD OF THE DISCLOSURE

The present disclosure relates to compositions and methods for improving glucose control, increasing lean body mass, reducing fat mass, treating obesity, diabetes, and/or treating liver dysfunction in a subject. More specifically, the disclosure relates to compositions comprising a GDF-8 inhibitor and a GLP-1 agonist and uses thereof, as well as to compositions comprising a GDF-8 inhibitor, an Activin A inhibitor, and a GLP-1 agonist and uses thereof.

### BACKGROUND

Obesity is a global problem for over a third of the world population. In the United States of America, the average obesity rate is over 20%. The costs of obesity-related illness are staggering, amounting to $190.2 billion, roughly 21% of annual medical costs in the U.S. Obesity is an epidemic disease characterized by chronic low-grade inflammation associated with dysfunctional (elevated) fat mass. Obesity is an important underlying risk factor for developing other diseases such as heart disease, stroke, and diabetes. Even a modest decrease in body weight (5-10% of initial body weight) lowers the risk for developing obesity-associated diseases such as heart disease and diabetes.

Diabetes mellitus is a chronic condition that is characterized by high blood sugar levels, and insulin resistance. If left untreated, the high blood sugar levels can lead to long-term complications including heart disease, stroke, diabetic retinopathy, and lower limb amputation. Treatment of diabetes involves controlling and reducing blood sugar levels and includes exercise and diet modification along with medications such as insulin and metformin.

Growth and differentiation factor-8 (GDF8, also known as myostatin), is a secreted ligand belonging to the transforming growth factor-β (TGF-β) superfamily of growth factors. GDF8 plays a central role in the development and maintenance of skeletal muscle, acting as a negative regulator of muscle mass. While the myostatin null mouse phenotype demonstrates the importance of GDF8 in the control of muscle size during development, muscle hypertrophy can also be elicited in adult muscle through inhibition of GDF8 with neutralizing antibodies, decoy receptors, or other antagonists. Administration of GDF8 neutralizing antibodies has been reported to result in muscle mass increases of between 10 and 30%. The increased muscle mass seen is due to increased fiber diameter as opposed to myofiber hyperplasia (fiber number). A number of studies have also reported increases in muscle strength or performance commensurate with increased size including twitch and tetanic force. Use of a cleavage resistant version of the GDF8 propeptide also leads to increased muscle size. Antibodies to GDF8 and therapeutic methods are disclosed in, e.g., U.S. Pat. No. 8,840,894. Anti-GDF8 antibodies are also mentioned in, e.g., U.S. Pat. Nos. 6,096,506; 7,320,789; 7,261,893; 7,807,159; 7,888,486; 7,635,760; 7,632,499; in US Patent Appl. Publ. Nos. 2006/0263354; 2007/0178095; 2008/0299126; 2010/0166764; 2009/0148436; and International Patent Appl. Publ. Nos. WO2004/037861; WO2007/047112; WO 2010/070094.

Activins belong to the transforming growth factor-beta (TGF-β) superfamily and exert a broad range of biological effects on cell proliferation, differentiation, metabolism, homeostasis, and apoptosis, as well as immune response and tissue repair. Activin A is a disulfide-linked homodimer (two beta-A chains) that binds to and activates heteromeric complexes of a type I (Act RI-A and Act RI-B) and a type II (Act RII-A and Act RII-B) serine-threonine kinase receptor.

Antibodies to Activin A and uses thereof are disclosed in, e.g., U.S. Pat. Nos. 8,309,082; 9,718,881; and International Patent Appl. Publ. No. WO2008/031061.

Compositions comprising an anti-GDF8 antibody and an anti-Activin A antibody and therapeutic methods are disclosed in, e.g., U.S. Pat. No. 8,871,209.

One of the approaches used for treating obesity and for glycemic control involves glucagon-like peptide (GLP)-1 receptor agonists that target the incretin pathway. Glucagon-like peptide (GLP)-1 is a peptide hormone secreted by intestinal enteroendocrine cells. Upon oral glucose administration, GLP-1 binds to its receptor leading to insulin secretion and a decrease in blood sugar levels (incretin effect). However, GLP-1 is rapidly inactivated and degraded by the enzyme dipeptidyl peptidase 4 (DPP4) and has a very short half-life of 1.5 minutes. Longer-acting derivatives of GLP-1 as well as GLP-1 receptor agonists including fusion proteins comprising GLP-1 have, therefore, been studied for diabetes control. GLP-1 analogues, fusion proteins and GLP-1 receptor agonists are disclosed, for example, in U.S. Pat. Nos. 7,452,966, 8,389,689, 8,496,149, 8,497,240, 8,557,769, 8,883,447, 8,895,694, 9,409,966, US20160194371, US20140024586, US20140073563, US20120148586, US20170114115, US20170112904, US20160361390, US20150313908, US20150259416, WO2017074715, WO2016127887, WO2015021871, WO2014113357, EP3034514, EP2470198, and EP2373681.

Because high fat mass is associated with such serious conditions as congestive heart failure, high blood pressure/hypertension, pulmonary embolism, osteoarthritis, lymphedema, gastro-esophageal reflux disease, chronic renal failure, cancer, fatty-liver disease, and even depression, there remains a need for therapies that reduce total fat and/or android fat mass in subjects. Furthermore, there remains a need for agents that treat obesity and diabetes while also treating related liver issues.

### BRIEF SUMMARY OF THE DISCLOSURE

In one aspect, the disclosure provides a composition comprising a Growth and Differentiation Factor-8 (GDF-8) inhibitor and an incretin inhibitor. In another aspect, the disclosure provides a composition comprising a GDF-8 inhibitor, an Activin A inhibitor, and an incretin inhibitor.

In another aspect, the disclosure provides a composition comprising a Growth and Differentiation Factor-8 (GDF-8) inhibitor and a Glucagon-like peptide-1 (GLP-1) agonist. In another aspect, the disclosure provides a composition comprising a GDF-8 inhibitor, an Activin A inhibitor, and a GLP-1 agonist.

In one embodiment of a composition according to the disclosure, the GDF-8 inhibitor is a GDF8-specific binding protein. In another embodiment, the GDF8 inhibitor is an antibody or antigen-binding fragment thereof that specifically binds GDF-8. In a further embodiment, the anti-GDF8 antibody or antigen-binding fragment thereof comprises the heavy chain complementarity determining regions (HCDRs) of a heavy chain variable region (HCVR) comprising SEQ ID NO:4, and the light chain complementarity determining regions (LCDRs) of a light chain variable region (LCVR) comprising SEQ ID NO:5. In still a further embodiment, the anti-GDF8 antibody or antigen-binding fragment thereof comprises heavy chain complementarity determining regions (HCDR1, HCDR2, and HCDR3) comprising the amino acid sequences of SEQ ID NO:6, SEQ ID NO:7, and SEQ ID NO:8, respectively, and three light chain complementarity determining regions (LCDR1, LCDR2, and LCDR3) comprising the amino acid sequences of SEQ ID NO:9, TTS, and SEQ ID NO: 11, respectively.

In one embodiment of a composition according to the disclosure, the Activin A inhibitor is an Activin A-specific binding protein. In another embodiment, the Activin A inhibitor is an antibody or antigen-binding fragment thereof that specifically binds Activin A. In a further embodiment, the anti-Activin A antibody or antigen-binding fragment thereof comprises the heavy chain complementarity determining regions (HCDRs) of a heavy chain variable region (HCVR) comprising SEQ ID NO:12, and the light chain complementarity determining regions (LCDRs) of a light chain variable region (LCVR) comprising SEQ ID NO:13. In still a further embodiment, the anti-Activin A antibody or antigen-binding fragment thereof comprises heavy chain complementarity determining regions (HCDR1, HCDR2, and HCDR3) comprising the amino acid sequences of SEQ ID NO: 14, SEQ ID NO: 15, and SEQ ID NO: 16, respectively, and three light chain complementarity determining regions (LCDR1, LCDR2, and LCDR3) comprising the amino acid sequences of SEQ ID NO: 17, GAS, and SEQ ID NO: 19, respectively.

In one embodiment of a composition according to the disclosure, the GLP-1 agonist is a GLP-1 receptor agonist. In another embodiment, the GLP-1 agonist is selected from the group consisting of Exenatide (long-acting), Dulaglutide, Liraglutide, Tirzepatide, and Semaglutide. In a further embodiment, the GLP-1 agonist is a GLP-1-specific binding protein. In still a further embodiment, the GLP-1 agonist is an antibody or antigen-binding fragment thereof that specifically binds GLP-1.

In one embodiment, a composition according to the disclosure is for use in improving glucose control, increasing lean body mass, reducing fat mass, treating obesity, treating diabetes, and/or treating liver issues associated with increased fat mass, obesity, and/or diabetes in a subject. In another embodiment, a composition according to the disclosure is for use in improving glucose control, increasing lean body mass, reducing fat mass, treating obesity, and/or treating diabetes, without exacerbating liver issues associated with increased fat mass, obesity, and/or diabetes in the subject.

In one aspect, the disclosure provides a method for improving glucose control, increasing lean body mass, reducing fat mass, reducing total cholesterol, reducing LDL cholesterol, increasing HDL cholesterol, treating obesity, treating diabetes, and/or treating liver issues associated with increased fat mass, obesity, and/or diabetes in a subject, comprising administering a composition comprising a GDF8 inhibitor and a GLP-1 agonist to the subject.

In another aspect, the disclosure provides a method for improving glucose control, increasing lean body mass, reducing fat mass, treating obesity, treating diabetes, and/or treating liver issues associated with increased fat mass, obesity, and/or diabetes in a subject, comprising administering a GDF8 inhibitor, an Activin A inhibitor, and a GLP-1 agonist to the subject.

In one embodiment of a method according to the disclosure, improving glucose control is demonstrated by/measured by reducing glycosylated hemoglobin (HbA1C). In another embodiment of a method according to the disclosure, the GDF8 inhibitor and the GLP-1 agonist, and the Activin A inhibitor, if present, are administered to the subject in a single composition. In still another embodiment, the GDF8 inhibitor and the GLP-1 agonist, and the Activin A inhibitor, if present, are administered to the subject in at least two separate compositions. In still another embodiment, the GDF8 inhibitor and the GLP-1 agonist, and the Activin A inhibitor, if present, are administered to the subject in three separate compositions.

In one embodiments of a method according to the disclosure, the GDF-8 inhibitor is a GDF8-specific binding protein. In another embodiment, the GDF8 inhibitor is an antibody or antigen-binding fragment thereof that specifically binds GDF-8. In a further embodiment, the anti-GDF8 antibody or antigen-binding fragment thereof comprises the heavy chain complementarity determining regions (HCDRs) of a heavy chain variable region (HCVR) comprising SEQ ID NO:4, and the light chain complementarity determining regions (LCDRs) of a light chain variable region (LCVR) comprising SEQ ID NO:5. In still a further embodiment, the anti-GDF8 antibody or antigen-binding fragment thereof comprises heavy chain complementarity determining regions (HCDR1, HCDR2, and HCDR3) comprising SEQ ID NO:6, SEQ ID NO:7, and SEQ ID NO:8, respectively, and three light chain complementarity determining regions (LCDR1, LCDR2, and LCDR3) comprising SEQ ID NO:9, TTS, and SEQ ID NO:11, respectively.

In one embodiment of a method according to the disclosure, the Activin A inhibitor is an Activin A-specific binding protein. In another embodiment, the Activin A inhibitor is an antibody or antigen-binding fragment thereof that specifically binds Activin A. In a further embodiment, the anti-Activin A antibody or antigen-binding fragment thereof comprises the heavy chain complementarity determining regions (HCDRs) of a heavy chain variable region (HCVR) comprising SEQ ID NO:12, and the light chain complementarity determining regions (LCDRs) of a light chain variable region (LCVR) comprising SEQ ID NO:13. In still a further embodiment, the anti-Activin A antibody or antigen-binding fragment thereof comprises heavy chain complementarity determining regions (HCDR1, HCDR2, and HCDR3) comprising SEQ ID NO: 14, SEQ ID NO: 15, and SEQ ID NO: 16, respectively, and three light chain complementarity determining regions (LCDR1, LCDR2, and LCDR3) comprising SEQ ID NO:17, GAS, and SEQ ID NO:19, respectively.

In one embodiment of a method according to the disclosure, the GLP-1 agonist is a GLP-1 receptor agonist. In another embodiment, the GLP-1 agonist is selected from the group consisting of Exenatide (long-acting), Dulaglutide, Liraglutide, Tirzepatide, and Semaglutide. In still another embodiment, the GLP-1 agonist is a GLP-1-specific binding protein. In still another embodiment, the GLP-1 agonist is an antibody or antigen-binding fragment thereof that specifically binds GLP-1.

In one embodiment of a method according to the disclosure, the subject, at 12 weeks from administration of the inhibitor(s) and the agonist, exhibits at least one parameter change selected from the group consisting of:
i) at least about 35% decrease in fat mass;
ii) at least about 6% increase in lean mass;
iii) at least about 15% decrease in fasting glucose;
iv) at least about 6% decrease in HbA1c;
v) at least about 14% decrease in LDL;
vi) at least about 14% increase in LDL;
vii) at least about 35% decrease in NEFA; and
viii) at least about 55% decrease in TG.
   In another embodiment, the subject, at 12 weeks from administration of the inhibitor(s) and the agonist, exhibits at least one parameter change selected from the group consisting of:
i) at least about 25% decrease in fasting glucose;
iv) at least about 25% decrease in HbA1c;
v) at least about 50% decrease in LDL;
vi) at least about 60% increase in LDL;
vii) at least about 50% decrease in NEFA; and
viii) at least about 65% decrease in TG.

In certain embodiments of a composition or method according to the disclosure, a single antigen-binding molecule comprises a GDF8-specific binding domain and an Activin A-specific binding domain. In one embodiment of this aspect of the disclosure, the antigen-binding molecule is a bispecific antibody comprising a first variable domain that specifically binds GDF8 and a second variable domain that specifically binds Activin A.

In one aspect, the present disclosure provides a use of a GDF-8 inhibitor and a GLP-1 agonist in the preparation of a medicament for improving glucose control, increasing lean body mass, reducing fat mass, treating obesity, treating diabetes, and/or treating liver issues associated with increased fat mass, obesity, and/or diabetes in a subject. In another aspect, the present disclosure provides a use of a GDF-8 inhibitor, an Activin A inhibitor, and a GLP-1 agonist in the preparation of a medicament for improving glucose control, increasing lean body mass, reducing fat mass, treating obesity, treating diabetes, and/or treating liver issues associated with increased fat mass, obesity, and/or diabetes in a subject.

Other embodiments of the present disclosure will become apparent from a review of the ensuing detailed description.

### BRIEF DESCRIPTION OF THE FIGURES

FIGS. 1A-1C show changes in body weight (FIG. 1A), fat mass (FIG. 1B), and lean mass (FIG. 1C) as measured by MRI overtime. Semaglutide via osmotic pump: 7 μg/day. IgG4^{P} (isotype control): 20 mg/kg. REGN1033 (α-GDF8): 10 mg/kg. REGN2477 (α-Activin A): 10 mg/kg.

FIGS. 2A and 2B show the percent change of (FIG. 2A) muscle and (FIG. 2B) adipose tissue weight compared to control at the end of the study. For final muscle weight (FIG. 2A), TA weight (% change vs. control) is shown in the left-hand bar graph, and quad weight (% change vs. control) is shown in the righthand bar graph. For final adipose tissue weight (FIG. 2B), subQ adipose weight (% change vs. control) is shown in the left-hand bar graph, and gonadal adipose weight (% change vs. control) is shown in the righthand bar graph.

FIGS. 3A-3C show the percent change of other peripheral organ weight (FIG. 3A pancreas, FIG. 3B heart, FIG. 3C spleen) compared to control at the end of the study.

FIGS. 4A-4C shows the fed blood glucose and insulin levels over time of the study. FIG. 4A fed blood glucose and FIG. 4B fed insulin, both over time; FIG. 4C fed glucagon at day 20.

FIGS. 5A and 5B show glucose tolerance measurement (day 23) and fasting glucose (day 23 and day 26). FIG. 5A shows blood glucose over time. FIG. 5B shows (left-hand bar graph) AUC glucose, (middle bar graph) 4 hr fasted blood glucose, (righthand bar graph) 6 hr fasted blood glucose.

FIGS. 6A and 6B show (FIG. 6A) pancreas weight (left-hand bar graph), beta cell mass (middle bar graph), and alpha cell mass (righthand bar graph) as determined by IHC for either insulin (beta cells) or glucagon (alpha cells). Representative picture for alpha cell staining shown in FIG. 6B.

FIGS. 7A-7F show measurement of circulating ALT (FIG. 7A), AST (FIG. 7B), Cholesterol (Chol) (FIG. 7C), Triglyceride (Trig) (FIG. 7D), and free fatty acids (NEFA) (FIG. 7E) at the end of the study. Measurement of triglycerides from liver tissue (FIG. 7F).

FIGS. 8A and 8B show (FIG. 8A) H&E staining of liver tissue and (FIG. 8B) histological quantification of lipid droplets.

FIGS. 9A and 9B show (FIG. 9A) liver α-SMA (smooth muscle actin) staining as indicator of fibrosis and (FIG. 9B) quantification of same.

FIG. 10 shows a timeline for an obese NHP study of the body weight, liver, and metabolic effects of adding on myostatin/activin A blockade onto GLP-1R agonism.

FIGS. 11A and 11B show that adding on anti-Myostatin treatment onto Semaglutide leads to greater weight loss than Semaglutide monotherapy. FIG. 11A shows the % change in body weight by group from day 0 (D0) on; FIG. 11B shows the % change in body weight by group from the start of antibody dosing (day 14 (D14)) on.

FIGS. 12A-12C show that adding anti-Myostatin treatment onto Semaglutide leads to greater fat loss than Semaglutide monotherapy, and that adding anti-Activin A on top of anti-Myostatin+Semaglutide also increases lean mass. Total mass (FIG. 12A), total fat mass (FIG. 12B), and total lean mass (FIG. 12C) are shown over time for each treatment group. The numbers in the line graphs constitute the percent change at week 12 (W12) from baseline.

FIGS. 13A-13C show that the triple combination of Semaglutide, anti-Myostatin treatment, and anti-Activin A treatment showed the greatest reduction in HbA1c % after 12 weeks of treatment. Fasting glucose (FIG. 13A), HbA1c (FIG. 13B), and insulin (FIG. 13C) are shown over time for each treatment group. Numbers above each group are % change from baseline at 12 weeks.

FIGS. 14A-14E show that the triple combination of Semaglutide, anti-Myostatin treatment, and anti-Activin A treatment showed the largest reduction in LDL, the largest increase in HDL after 12 weeks of treatment. Total cholesterol (FIG. 14A), LDL (FIG. 14B), HDL (FIG. 14C), NEFA (FIG. 14D), and TG (FIG. 14E) are shown over time for each treatment group. Numbers above each group are % change from baseline at 12 weeks.

FIGS. 15A-15C show the AST (FIG. 15A), ALT (FIG. 15B), and AST/ALT ratio (FIG. 15C) for the different treatment groups through week 12.

FIGS. 16A and 16B show the total energy intake (7-day average) (FIG. 16A) and the cumulative energy intake (FIG. 16B) over time (through week 12) for the different treatment groups.

FIGS. 17A and 17B show the total water intake (7-day average) (FIG. 17A) and the cumulative water intake (FIG. 17B) over time (through week 12) for the different treatment groups.

FIG. 18 shows that myostatin (GDF8) and activin A blockade synergistically increases muscle mass in mice.

FIGS. 19A-19D show the changes in thigh muscle volume in postmenopausal women (FIG. 19A), in total lean mass in obese non-human primates (FIG. 19B), in android fat mass in postmenopausal women (FIG. 19C), and in total fat mass in obese non-human primates (FIG. 19D) over time, each group receiving various treatments.

FIGS. 20A and 20B show raw energy expenditure (FIG. 20A) and energy expenditure per kg lean mass (FIG. 20B) in obese non-human primates receiving various treatment combinations.

FIGS. 21A and 21B show the energy expenditure vs. total lean mass for the non-combination treatment groups (vehicle, semaglutide, trevogrumab+garetosmab) and for the combination treatment groups (semaglutide+trevogrumab, semaglutide+trevogrumab+garetosmab) (FIG. 21A above for both sets of treatment groups, below left for combination treatment groups, below right for non-combination treatment groups); and the energy expenditure vs. lean mass percent change from baseline for the non-combination treatment groups (vehicle, semaglutide, trevogrumab+garetosmab) and for the combination treatment groups (semaglutide+trevogrumab, semaglutide+trevogrumab+garetosmab) (FIG. 21B above for both sets of treatment groups, below left for non-combination treatment groups, below right for combination treatment groups).

FIGS. 22A-22F show the measurements of HbA1c (FIG. 22A), LDL-C (FIG. 22B), ApoB (FIG. 22C), HbA1c change vs. baseline (FIG. 22D), LDL-C change vs. baseline (FIG. 22E), ApoB change vs. baseline (FIG. 22F) in the obese non-human primate treatment groups of FIGS. 21A and 21B.

FIG. 23 schematically depicts the study design for the obese non-human primate study of the body weight, liver, and metabolic effects of adding on myostatin/activin A blockade onto GLP-1R agonism.

### DETAILED DESCRIPTION

Before the present disclosure is described, it is to be understood that this disclosure is not limited to particular methods and experimental conditions described, as such methods and conditions may vary. It is also to be understood that the terminology used herein is for the purpose of describing particular embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. As used herein, the term “about,” when used in reference to a particular recited numerical value, means that the value may vary from the recited value by no more than 1%. For example, as used herein, the expression “about 100” includes 99 and 101 and all values in between (e.g., 99.1, 99.2, 99.3, 99.4, etc.). Ranges can be expressed herein as from “about” or “approximately” one particular value and/or to “about” or “approximately” another particular value. When such a range is expressed, another embodiment includes from the one particular value and/or to the other particular value.

By “comprising” or “containing” or “including” is meant that at least the named compound, element, particle, or method step is present in the composition or article or method, but does not exclude the presence of other compounds, materials, particles, or method steps, even if the other such compounds, material, particles, or method steps have the same function as what is named.

### Antigen-Specific Binding Proteins

The present disclosure relates to compositions comprising agonists and antigen-specific binding proteins. More specifically, in certain embodiments, the present disclosure provides compositions comprising a GLP-1 agonist and a GDF-8-specific binding protein, as well as compositions comprising a GLP-1 agonist and a GDF-8-specific binding protein and an Activin A-specific binding protein.

As used herein, the expression “antigen-specific binding protein” means a protein comprising at least one domain that specifically binds a particular antigen. Exemplary categories of antigen-specific binding proteins include antibodies, antigen-binding portions of antibodies, peptides that specifically interact with a particular antigen (e.g., peptibodies), receptor molecules that specifically interact with a particular antigen, and proteins comprising a ligand-binding portion of a receptor that specifically binds a particular antigen.

The present disclosure includes antigen-specific binding proteins that specifically bind GDF-8, i.e., “GDF-8-specific binding proteins”. The term “GDF-8” (also referred to as “growth and differentiation factor-8” and “myostatin”) means the protein having the amino acid sequence of SEQ ID NO:25 (mature protein) (SEQ ID NO:1). According to the present disclosure, GDF8-specific binding proteins specifically bind GDF-8 but do not bind other ActRIIB ligands such as GDF3, BMP2, BMP4, BMP7, BMP9, BMP10, GDF11, Activin A, Activin B, Activin AB, Nodal, etc.

The present disclosure also includes antigen-specific binding proteins that specifically bind Activin A, i.e., “Activin A-specific binding proteins”. Activins are homo- and hetero-dimeric molecules comprising βA and/or βB subunits. The βA subunit has the amino acid sequence of SEQ ID NO:2 and the βB subunit has the amino acid sequence of SEQ ID NO:3. Activin A is a homodimer of two βA subunits; Activin B is a homodimer of two βB subunits; and Activin βB is a heterodimer of one βA subunit and one βB subunit. An Activin A-specific binding protein may be an antigen-specific binding protein that specifically binds the βA subunit. Since the βA subunit is found in both Activin A and Activin AB molecules, an “Activin A-specific binding protein” can be an antigen-specific binding protein that specifically binds Activin A as well as Activin AB (by virtue of its interaction with the βA subunit). Therefore, according to the present disclosure, an Activin A-specific binding protein specifically binds Activin A, or Activin A and Activin AB, but does not bind other ActRIIB ligands such as Activin B, GDF3, GDF8, BMP2, BMP4, BMP7, BMP9, BMP10, GDF11, Nodal, etc.

In the context of the present disclosure, molecules such as ActRIIB-Fc (e.g., “ACE-031”), which comprise the ligand-binding portion of the ActRIIB receptor, are not considered “GDF8-specific binding proteins” or “Activin A-specific binding proteins”, because such molecules bind multiple ligands besides GDF8, Activin A, and Activin AB.

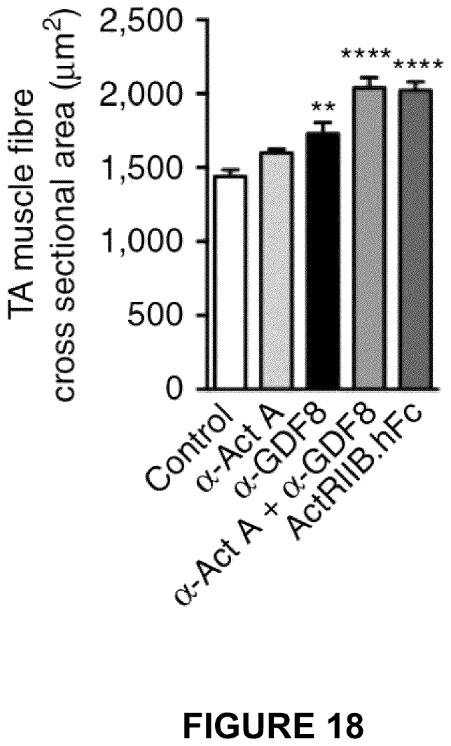

In one embodiment, the myostatin (GDF8) and activin A inhibition can alternatively be provided by an ActRIlb-Fc molecule or an antibody that binds to ActRIIB. When male CB17 SCID mice were treated with anti-activin A antibody, anti-GDF8 antibody, anti-activin A antibody+anti-GDF8 antibody, or ActRIIB.hFc, the increase in TA muscle for the anti-GDF8+anti-Activin A combination were substantially greater than the increases in these parameters observed in the anti-GDF8 or anti-Activin A monotherapy subjects. The ActRIIB-Fc-treated animals also showed substantially greater increase in muscle mass (FIG. 18). In another embodiment, the antibody that binds to ActRIIB is bimagrumab.

The present disclosure includes antigen-specific binding proteins that specifically bind GLP-1 and/or GLP-1R, i.e., “GLP-1-specific binding proteins”. The term “GLP-1R” refers to the glucagon-like peptide 1 receptor and includes recombinant GLP-1R protein or a fragment thereof. GLP-1R has a sequence of 463 residues (NCBI accession no. NP_002053, SEQ ID NO:20). Donnelly, 2011, Br J Pharmacol 166(1):27-41 (2011). Glucagon-like peptide 1 (GLP-1) is a 31-amino acid peptide hormone released from intestinal L cells following nutrient consumption. The binding of GLP-1 to GLP-1R potentiates glucose-induced secretion of insulin from pancreatic beta cells, increases insulin expression, inhibits beta-cell apoptosis, promotes beta-cell neogenesis, reduces glucagon secretion, delays gastric emptying, promotes satiety and increases peripheral glucose disposal.

### Antigen-Binding Molecules With Two Different Antigen-Specific Binding Domains

The present disclosure also includes antigen-binding molecules comprising two different antigen-specific binding domains. In particular, the present disclosure includes antigen-binding molecules comprising a GDF8-specific binding domain and an Activin A-specific binding domain. The term “antigen-specific binding domain,” as used herein, includes polypeptides comprising or consisting of: (i) an antigen-binding fragment of an antibody molecule, (ii) a peptide that specifically interacts with a particular antigen (e.g., a peptibody), and/or (iii) a ligand-binding portion of a receptor that specifically binds a particular antigen. For example, the present disclosure includes bispecific antibodies with one arm comprising a first heavy chain variable region/light chain variable region (HCVR/LCVR) pair that specifically binds GDF8 and another arm comprising a second HCVR/LCVR pair that specifically binds Activin A. Thus, in a composition comprising a GDF-8-specific binding protein and an Activin A-specific binding protein (and a GLP-1 agonist) may, in fact, comprise a single binding protein comprising both a GDF8-specific binding domain and an Activin A-specific binding domain.

### Specific Binding

The term “specifically binds” or the like, as used herein, means that an antigen-specific binding protein, or an antigen-specific binding domain, forms a complex with a particular antigen characterized by a dissociation constant (K_{D}) of 500 pM or less, and does not bind other unrelated antigens under ordinary test conditions. “Unrelated antigens” are proteins, peptides or polypeptides that have less than 95% amino acid identity to one another. Methods for determining whether two molecules specifically bind one another are well known in the art and include, for example, equilibrium dialysis, surface plasmon resonance, and the like. For example, an antigen-specific binding protein or an antigen-specific binding domain, as used in the context of the present disclosure, includes molecules that bind a particular antigen (e.g., GDF-8, or Activin A and/or AB, or GLP-1/GLP-1R) or a portion thereof with a K_{D} of less than about 500 pM, less than about 400 pM, less than about 300 PM, less than about 200 pM, less than about 100 pM, less than about 90 pM, less than about 80 pM, less than about 70 pM, less than about 60 pM, less than about 50 pM, less than about 40 pM, less than about 30 pM, less than about 20 pM, less than about 10 pM, less than about 5 pM, less than about 4 pM, less than about 2 pM, less than about 1 pM, less than about 0.5 pM, less than about 0.2 pM, less than about 0.1 pM, or less than about 0.05 PM, as measured in a surface plasmon resonance assay.

As used herein, an antigen-specific binding protein or antigen-specific binding domain “does not bind” to a specified molecule if the protein or binding domain, when tested for binding to the molecule at 25° C. in a surface plasmon resonance assay, exhibits a K_{D} of greater than 1000 pM, or fails to exhibit any binding in such an assay or equivalent thereof.

The term “surface plasmon resonance”, as used herein, refers to an optical phenomenon that allows for the analysis of real-time interactions by detection of alterations in protein concentrations within a biosensor matrix, for example using the BIAcore™ system (Biacore Life Sciences division of GE Healthcare, Piscataway, NJ).

The term “K_{D}”, as used herein, means the equilibrium dissociation constant of a particular protein-protein interaction (e.g., antibody-antigen interaction). Unless indicated otherwise, the K_{D} values disclosed herein refer to K_{D} values determined by surface plasmon resonance assay at 25° C.

### Antibodies and Antigen-Binding Fragments of Antibodies

As indicated above, an antigen-specific binding protein can comprise or consist of an antibody or antigen-binding fragment of an antibody. Furthermore, in the case of antigen-binding molecules comprising two different antigen-specific binding domains, one or both of the antigen-specific binding domains may comprise or consist of an antigen-binding fragment of an antibody.

The term “antibody”, as used herein, is intended to refer to immunoglobulin molecules comprising four polypeptide chains, two heavy (H) chains and two light (L) chains inter-connected by disulfide bonds, as well as multimers thereof (e.g., IgM). Each heavy chain comprises a heavy chain variable region (abbreviated herein as HCVR or V_{H}) and a heavy chain constant region. The heavy chain constant region comprises three domains, C_{H}1, C_{H}2 and C_{H}3. Each light chain comprises a light chain variable region (abbreviated herein as LCVR or V_{L}) and a light chain constant region. The light chain constant region comprises one domain (C_{L}1). The V_{H} and V_{L} regions can be further subdivided into regions of hypervariability, termed complementarity determining regions (CDRs), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. In different embodiments of the disclosure, the FRs of the antibodies of the disclosure (or antigen-binding portion thereof) may be identical to the human germline sequences, or may be naturally or artificially modified. An amino acid consensus sequence may be defined based on a side-by-side analysis of two or more CDRs.

The term “antibody,” as used herein, also includes antigen-binding fragments of full antibody molecules. The terms “antigen-binding portion” of an antibody, “antigen-binding fragment” of an antibody, and the like, as used herein, include any naturally occurring, enzymatically obtainable, synthetic, or genetically engineered polypeptide or glycoprotein that specifically binds an antigen to form a complex. Antigen-binding fragments of an antibody may be derived, e.g., from full antibody molecules using any suitable standard techniques such as proteolytic digestion or recombinant genetic engineering techniques involving the manipulation and expression of DNA encoding antibody variable and optionally constant domains. Such DNA is known and/or is readily available from, e.g., commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

Non-limiting examples of antigen-binding fragments include: (i) Fab fragments; (ii) F(ab′)2 fragments; (iii) Fd fragments; (iv) Fv fragments; (v) single-chain Fv (scFv) molecules; (vi) dAb fragments; and (vii) minimal recognition units consisting of the amino acid residues that mimic the hypervariable region of an antibody (e.g., an isolated complementarity determining region (CDR) such as a CDR3 peptide), or a constrained FR3-CDR3-FR4 peptide. Other engineered molecules, such as domain-specific antibodies, single domain antibodies, domain-deleted antibodies, chimeric antibodies, CDR-grafted antibodies, diabodies, triabodies, tetrabodies, minibodies, nanobodies (e.g., monovalent nanobodies, bivalent nanobodies, etc.), small modular immunopharmaceuticals (SMIPs), and shark variable IgNAR domains, are also encompassed within the expression “antigen-binding fragment,” as used herein.

An antigen-binding fragment of an antibody will typically comprise at least one variable domain. The variable domain may be of any size or amino acid composition and will generally comprise at least one CDR which is adjacent to or in frame with one or more framework sequences. In antigen-binding fragments having a V_{H} domain associated with a V_{L} domain, the V_{H} and V_{L} domains may be situated relative to one another in any suitable arrangement. For example, the variable region may be dimeric and contain V_{H}-V_{H}, V_{H}-V_{L} or V_{L}-V_{L} dimers. Alternatively, the antigen-binding fragment of an antibody may contain a monomeric V_{H} or V_{L} domain.

In certain embodiments, an antigen-binding fragment of an antibody may contain at least one variable domain covalently linked to at least one constant domain. Non-limiting, exemplary configurations of variable and constant domains that may be found within an antigen-binding fragment of an antibody of the present disclosure include: (i) V_{H}-C_{H}1; (ii) V_{H}-C_{H}2; (iii) V_{H}-C_{H}3; (iv) V_{H}-C_{H}1-C_{H}2; (V) V_{H}-C_{H}1-C_{H}2-C_{H}3; (vi) V_{H}-C_{H}2-C_{H}3; (vii) V_{H}-C_{L}; (viii) V_{L}-C_{H}1; (ix) V_{L}-C_{H}2; (x) V_{L}-C_{H}3; (xi) V_{L}-C_{H}1-C_{H}2; (xii) V_{L}-C_{H}1-C_{H}2-C_{H}3; (xiii) V_{L}-C_{H}2-C_{H}3; and (xiv) V_{L}-C_{L}. In any configuration of variable and constant domains, including any of the exemplary configurations listed above, the variable and constant domains may be either directly linked to one another or may be linked by a full or partial hinge or linker region. A hinge region may consist of at least 2 (e.g., 5, 10, 15, 20, 40, 60 or more) amino acids which result in a flexible or semi-flexible linkage between adjacent variable and/or constant domains in a single polypeptide molecule.

Moreover, an antigen-binding fragment of an antibody of the present disclosure may comprise a homo-dimer or hetero-dimer (or other multimer) of any of the variable and constant domain configurations listed above in non-covalent association with one another and/or with one or more monomeric V_{H} or V_{L} domain (e.g., by disulfide bond(s)).

The molecules of the present disclosure may comprise or consist of human antibodies and/or recombinant human antibodies, or fragments thereof. The term “human antibody”, as used herein, includes antibodies having variable and constant regions derived from human germline immunoglobulin sequences. Human antibodies may nonetheless include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody”, as used herein, is not intended to include antibodies in which CDR sequences derived from the germline of another mammalian species, such as a mouse, have been grafted onto human framework sequences.

The molecules of the present disclosure may comprise or consist of recombinant human antibodies or antigen-binding fragments thereof. The term “recombinant human antibody”, as used herein, is intended to include all human antibodies that are prepared, expressed, created or isolated by recombinant means, such as antibodies expressed using a recombinant expression vector transfected into a host cell (described further below), antibodies isolated from a recombinant, combinatorial human antibody library (described further below), antibodies isolated from an animal (e.g., a mouse) that is transgenic for human immunoglobulin genes (see e.g., Taylor, et al., (1992) Nucl. Acids Res. 20:6287-6295) or antibodies prepared, expressed, created or isolated by any other means that involves splicing of human immunoglobulin gene sequences to other DNA sequences. Such recombinant human antibodies have variable and constant regions derived from human germline immunoglobulin sequences. In certain embodiments, however, such recombinant human antibodies are subjected to in vitro mutagenesis (or, when an animal transgenic for human Ig sequences is used, in vivo somatic mutagenesis) and thus the amino acid sequences of the V_{H} and V_{L} regions of the recombinant antibodies are sequences that, while derived from and related to human germline V_{H} and V_{L} sequences, may not naturally exist within the human antibody germline repertoire in vivo.

All amino acid abbreviations used in this disclosure are those accepted by the United States Patent and Trademark Office as set forth in 37 C.F.R. § 1.822 (B)(J). The amino acid sequence of an antibody or antigen-binding fragment thereof can be numbered using any known numbering schemes, including those described by Kabat, et al., (“Kabat” numbering scheme); Al-Lazikani, et al., 1997, J. Mol. Biol. 273:927-948 (“Chothia” numbering scheme); MacCallum, et al., 1996, J. Mol. Biol. 262:732-745 (“Contact” numbering scheme); Lefranc, et al., 2003, Dev. Comp. Immunol. 27:55-77 (“IMGT” numbering scheme); and Honegge and Pluckthun, 2001, J. Mol. Biol. 309:657-70 (“AHo” numbering scheme).

An “agonist” antibody or antigen-binding fragment thereof as used herein is an antibody or fragment that increases or enhances at least one biological activity of the antigen, for example, GLP-1 and/or GLP-1-R. Such increase or enhancement may be mediated by the antibody itself or, if the antibody is part of an antibody drug conjugate or antibody-tethered drug conjugate, by the payload or linker-payload. For example, the agonist antibody or fragment may elicit stimulation of the adenylate cyclase pathway resulting in increased synthesis of cyclic AMP and release of insulin if the cell is a mammalian pancreatic beta cell. Other biological activities of GLP-1R may be cAMP-dependent activation of protein kinase A (PKA) and/or cAMP-regulated guanine nucleotide exchange factor 2 (Epac2). An agonist antibody or fragment may also reduce glucose levels or reduce body weight upon administration to a subject.

### Anti-GDF8 Antibodies and Antigen-Binding Fragments Thereof

In certain specific embodiments of the present disclosure, the GDF-8 inhibitor is a GDF8-specific binding protein, and the protein, or the GDF8-specific binding domain, comprises or consists of an anti-GDF8 antibody or antigen-binding fragment thereof. Anti-GDF8 antibodies are mentioned in, e.g., U.S. Pat. Nos. 6,096,506; 7,320,789; 7,261,893; 7,807,159; 7,888,486; 7,635,760; 7,632,499; in US Patent Appl. Publ. Nos. 2007/0178095; 2010/0166764; 2009/0148436; and International Patent Appl. Publ. No. WO 2010/070094. Anti-GDF8 antibodies are also described in U.S. patent application Ser. No. 13/115,170, filed on May 25, 2011, and published as US 20110293630, including the antibodies designated 8D12, H4H1657N2, and H4H1669P. In one embodiment, the anti-GDF8 antibody is REGN1033, also known as H4H1657N2. Any of the anti-GDF8 antibodies mentioned and/or described in any of the foregoing patents or publications, or antigen-binding fragments thereof, can be used in the context of the present disclosure, so long as such antibodies and/or antigen-binding fragments “specifically bind” GDF8, as that expression is defined herein.

In one embodiment, the anti-GDF8 antibody or antigen-binding fragment thereof comprises the heavy chain complementarity determining regions (HCDRs) of a heavy chain variable region (HCVR) comprising SEQ ID NO:4, and the light chain complementarity determining regions (LCDRs) of a light chain variable region (LCVR) comprising SEQ ID NO:5. In another embodiment, the anti-GDF8 antibody or antigen-binding fragment thereof comprises heavy chain complementarity determining regions (HCDR1, HCDR2, and HCDR3) comprising SEQ ID NO:6, SEQ ID NO:7, and SEQ ID NO:8, respectively, and three light chain complementarity determining regions (LCDR1, LCDR2, and LCDR3) comprising SEQ ID NO:9, TTS, and SEQ ID NO: 11, respectively.

### Anti-Activin A Antibodies and Antigen-Binding Fragments Thereof

In certain specific embodiments of the present disclosure, the Activin A inhibitor is an Activin A-specific binding protein, and the protein, or the Activin A-specific binding domain, comprises or consists of an antibody or antigen-binding fragment thereof that specifically binds Activin A. In certain embodiments, the Activin A-specific binding protein specifically binds the βA subunit. An antigen-specific binding protein that specifically binds the βA subunit may recognize both Activin A (βA/βA homodimer) and Activin AB (βA/βB heterodimer). Thus, according to the present disclosure, an Activin A-specific binding protein may bind both Activin A and Activin AB (but not Activin B). Anti-Activin A antibodies are mentioned in, e.g., US Patent Appl. Publ. No 2009/0234106. In one embodiment, the anti-Activin A antibody is REGN2477, also referred to as H4H10446P2. In another embodiment, the anti-Activin A antibody is REGN2376, also referred to as H4H10430P. Another anti-Activin A antibody is designated “MAB3381” and is available commercially from R&D Systems, Inc, Minneapolis, MN. MAB3381 specifically binds Activin A (homodimer) as well as Activin AB (heterodimer). Any of the aforementioned anti-Activin A antibodies, or antigen-binding fragments thereof, can be used in the context of the present disclosure, so long as such antibodies and/or antigen-binding fragments “specifically bind” Activin A and/or Activin AB, as defined herein.

In one embodiment, the anti-Activin A antibody or antigen-binding fragment thereof comprises the heavy chain complementarity determining regions (HCDRs) of a heavy chain variable region (HCVR) comprising SEQ ID NO: 12, and the light chain complementarity determining regions (LCDRs) of a light chain variable region (LCVR) comprising SEQ ID NO:13. In another embodiment, the anti-Activin A antibody or antigen-binding fragment thereof comprises heavy chain complementarity determining regions (HCDR1, HCDR2, and HCDR3) comprising SEQ ID NO: 14, SEQ ID NO: 15, and SEQ ID NO: 16, respectively, and three light chain complementarity determining regions (LCDR1, LCDR2, and LCDR3) comprising SEQ ID NO:17, GAS, and SEQ ID NO: 19, respectively.

### Glucagon-Like Peptide (GLP)-1 Agonists/Glucagon-Like Peptide (GLP)-1 Receptor Agonists

Incretins are gut-derived hormones that are released in response to the ingestion of nutrients and that stimulate insulin secretion together with hyperglycemia. In some embodiments of the compositions and methods according to the disclosure, incretin inhibitors are used in combination with other agents (e.g., GDF-8 inhibitor, or GDF-8 inhibitor+Activin A inhibitor). In further embodiments of the compositions and methods according to the disclosure, GLP-1 agonists or dipeptidyl peptidose IV (DPP-4) inhibitors are used in combination with other agents (e.g., GDF-8 inhibitor, or GDF-8 inhibitor+Activin A inhibitor).

The term “GLP-1”, also called as “glucagon-like peptide 1”, refers to the 31-amino acid peptide hormone released from intestinal L cells following nutrient consumption. GLP-1 binds to GLP-1 receptor and potentiates the glucose-induced secretion of insulin from pancreatic beta cells, increases insulin expression, inhibits beta-cell apoptosis, promotes beta-cell neogenesis, reduces glucagon secretion, delays gastric emptying, promotes satiety and increases peripheral glucose disposal.

As used herein, the term “GLP-1 agonist” refers to a compound that promotes, upregulates, or simulates the activity of GLP-1. GLP-1 agonists can activate GLP-1R and include GLP-1 mimetics, peptides variants, antibodies (including antibodies tethered to ligands), and fusion proteins. GLP-1 agonists include GLP-1 receptor agonists (GLP-1 RAs). The GLP-1 agonists described/used herein are GLP-1 receptor agonists. Indeed, for the purposes of the instant disclosure, the expressions “GLP-1 agonist” and “GLP-1R agonist” are used interchangeably. As used herein, the term “GLP-1 receptor agonist” refers to a compound that binds to GLP-1 receptor. GLP-1 receptor agonists increase glucose-dependent insulin secretion and decrease inappropriate glucagon secretion, delay gastric emptying, and increase satiety (Trujillo, et al., 2021, Ther Adv Endocrinol Metab 12:1-15). GLP-1 agonists may, for example, be selected from small molecule and peptide GLP-1R agonists and allosteric modulators (Graaf, et al., 2016, Pharmacol Rev 68:954-1013).

GLP-1 agonists for use in the instant disclosure include peptide agonists now on the market. In certain embodiments, the GLP-1 agonists mimic the action of glucagon-like peptide 1. Known GLP-1 receptor agonists include Albiglutide, Exenatide (short-acting and long-acting), Efpeglenatide, ITCA650, Lixisenatide, Liraglutide, Dulaglutide, and Semaglutide. In certain embodiments, the GLP-1 agonist is selected from the group consisting of Exenatide (long-acting), Dulaglutide, Liraglutide, and Semaglutide. In a further embodiment of a composition or method according to the disclosure, the GLP-1 agonist is Semaglutide. Semaglutide (sold under the brand name Ozempic, among others) is a glucagon-like peptide-1 receptor agonist that increases the production and secretion of insulin, thus increasing sugar metabolism. In one embodiment, the GLP-1 agonist for us in a method or composition according to the disclosure is a modified peptide drug, for example, Tirzepatide, that activates both the Glucagon-like peptide-1 (GLP-1) and glucose-dependent insulinotropic polypeptide (GIP) receptors.

In another embodiment, the GPL-1 agonist/receptor agonist for use in a composition or method according to the disclosure is an antibody or antigen-binding fragment thereof that specifically binds GLP-1.

In certain embodiments, the GLP-1 agonist for use in compositions and methods according to the invention is an antibody-drug conjugate (ADC) that specifically binds the glucagon-like peptide 1 receptor (GLP-1R) protein. In a further embodiment, the antibody or antigen-binding fragment thereof of the ADC specifically targets the extracellular domain of GLP-1R, with a GLP-1 peptidomimetic functionally activating GLP-1R.

An antibody-tethered drug conjugate (ATDC) or antibody-drug conjugate (ADC) refers to an antibody or antigen-binding fragments thereof tethered, by a linker or without a linker, to a payload (e.g., a GLP-1 peptidimimetic). An antibody-payload conjugate refers to such an antibody or fragment linked to a payload whereas an antibody-linker-payload conjugate refers to an antibody or fragment conjugated to a payload via a linker. An antibody or antigen-binding fragment referred to herein includes embodiments wherein said antibody or fragment is be conjugated to a payload or linker-payload.

### Bioequivalents

In certain embodiments, the GDF-8 inhibitor, Activin A inhibitor, and/or GLP-1 agonist of the present disclosure encompass proteins having amino acid sequences that vary from those of the described GDF-8 inhibitor, Activin A inhibitor, and/or GLP-1 agonist, but that retain the ability to bind GDF-8, Activin A, and GLP-1, respectively. Such variants comprise one or more additions, deletions, or substitutions of amino acids when compared to parent sequence, but exhibit biological activity that is essentially equivalent to that of the described GDF-8 inhibitor, Activin A inhibitor, and/or GLP-1 agonist.

Two proteins are considered bioequivalent if, for example, they are pharmaceutical equivalents or pharmaceutical alternatives whose rate and extent of absorption do not show a significant difference when administered at the same molar dose under similar experimental conditions, either single dose or multiple doses. Some proteins will be considered equivalents or pharmaceutical alternatives if they are equivalent in the extent of their absorption but not in their rate of absorption and yet may be considered bioequivalent because such differences in the rate of absorption are intentional and are reflected in the labeling, are not essential to the attainment of effective body drug concentrations on, e.g., chronic use, and are considered medically insignificant for the particular drug product studied.

In one embodiment, two GDF8 inhibitor, Activin A inhibitor, or GLP-1 agonist proteins are bioequivalent, if there are no clinically meaningful differences in their safety, purity, or potency.

In one embodiment, two GDF8 inhibitor, Activin A inhibitor, or GLP-1 agonist proteins are bioequivalent, if a patient can be switched one or more times between the reference product and the biological product without an expected increase in the risk of adverse effects, including a clinically significant change in immunogenicity, or diminished effectiveness, as compared to continued therapy without such switching.

In one embodiment, two GDF8 inhibitor, Activin A inhibitor, or GLP-1 agonist proteins are bioequivalent, if they both act by a common mechanism or mechanisms of action for the condition or conditions of use, to the extent that such mechanisms are known.

Bioequivalence may be demonstrated by in vivo and/or in vitro methods. Bioequivalence measures include, e.g., (a) an in vivo test in humans or other mammals, in which the concentration of the protein or its metabolites is measured in blood, plasma, serum, or other biological fluid as a function of time; (b) an in vitro test that has been correlated with and is reasonably predictive of human in vivo bioavailability data; (c) an in vivo test in humans or other mammals in which the appropriate acute pharmacological effect of the protein (or its target) is measured as a function of time; and (d) in a well-controlled clinical trial that establishes safety, efficacy, or bioavailability or bioequivalence of an antigen-binding protein.

Bioequivalent variants of the GDF8 inhibitor, Activin A inhibitor, and/or GLP-1 agonist proteins of the disclosure may be constructed by, for example, making various substitutions of residues or sequences or deleting terminal or internal residues or sequences not needed for biological activity. For example, cysteine residues not essential for biological activity can be deleted or replaced with other amino acids to prevent formation of unnecessary or incorrect intramolecular disulfide bridges upon renaturation. In other contexts, bioequivalent proteins may include variants comprising amino acid changes, which modify the glycosylation characteristics of the proteins, e.g., mutations that eliminate or remove glycosylation.

### Pharmaceutical Compositions and Methods of Administration

The present disclosure includes pharmaceutical compositions comprising a GDF8 inhibitor and a GLP-1 agonist. The present disclosure also includes pharmaceutical compositions comprising a GDF8 inhibitor, an Activin A inhibitor, and a GLP-1 agonist. The pharmaceutical compositions of the disclosure are formulated with suitable carriers, excipients, and other agents that provide suitable transfer, delivery, tolerance, and the like. A multitude of appropriate formulations can be found in, e.g., Remington's Pharmaceutical Sciences, Mack Publishing Company, Easton, PA. Suitable formulations include, for example, powders, pastes, ointments, jellies, waxes, oils, lipids, lipid (cationic or anionic) containing vesicles (such as LIPOFECTIN™), DNA conjugates, anhydrous absorption pastes, oil-in-water and water-in-oil emulsions, emulsions carbowax (polyethylene glycols of various molecular weights), semi-solid gels, and semi-solid mixtures containing carbowax. Additional suitable formulations are also described in Powell, et al., “Compendium of excipients for parenteral formulations” PDA (1998) J Pharm Sci Technol 52:238-311.

Various delivery systems are known and can be used to administer the pharmaceutical compositions of the present disclosure, e.g., encapsulation in liposomes, microparticles, microcapsules, recombinant cells capable of expressing the mutant viruses, receptor mediated endocytosis (see, e.g., Wu, et al., 1987, J. Biol. Chem. 262:4429-4432). Methods of administration include, but are not limited to, intradermal, intramuscular, intraperitoneal, intravenous, subcutaneous, intranasal, epidural, and oral routes. The compositions may be administered by any convenient route, for example by infusion or bolus injection, by absorption through epithelial or mucocutaneous linings (e.g., oral mucosa, rectal and intestinal mucosa, etc.) and may be administered together with other biologically active agents.

A pharmaceutical composition of the present disclosure can be delivered subcutaneously or intravenously with a standard needle and syringe. In addition, with respect to subcutaneous delivery, a pen delivery device readily has applications in delivering a pharmaceutical composition of the present disclosure. Such a pen delivery device can be reusable or disposable. A reusable pen delivery device generally utilizes a replaceable cartridge that contains a pharmaceutical composition. Once all of the pharmaceutical composition within the cartridge has been administered and the cartridge is empty, the empty cartridge can readily be discarded and replaced with a new cartridge that contains the pharmaceutical composition. The pen delivery device can then be reused. In a disposable pen delivery device, there is no replaceable cartridge. Rather, the disposable pen delivery device comes prefilled with the pharmaceutical composition held in a reservoir within the device. Once the reservoir is emptied of the pharmaceutical composition, the entire device is discarded.

Numerous reusable pen and autoinjector delivery devices have applications in the subcutaneous delivery of a pharmaceutical composition of the present disclosure. Examples include, but are not limited to AUTOPEN™ (Owen Mumford, Inc., Woodstock, UK), DISETRONIC™ pen (Disetronic Medical Systems, Bergdorf, Switzerland), HUMALOG MIX 75/25™ pen, HUMALOG™ pen, HUMALIN 70/30™ pen (Eli Lilly and Co., Indianapolis, IN), NOVOPEN™ I, II and III (Novo Nordisk, Copenhagen, Denmark), NOVOPEN JUNIOR™ (Novo Nordisk, Copenhagen, Denmark), BD™ pen (Becton Dickinson, Franklin Lakes, NJ), OPTIPEN™, OPTIPEN PRO™, OPTIPEN STARLET™, and OPTICLIK™ (sanofi-aventis, Frankfurt, Germany), to name only a few. Examples of disposable pen delivery devices having applications in subcutaneous delivery of a pharmaceutical composition of the present disclosure include, but are not limited to the SOLOSTAR™ pen (sanofi-aventis), the FLEXPEN™ (Novo Nordisk), and the KWIKPEN™ (Eli Lilly), the SURECLICK™ Autoinjector (Amgen, Thousand Oaks, CA), the PENLET™ (Haselmeier, Stuttgart, Germany), the EPIPEN (Dey, L.P.), and the HUMIRA™ Pen (Abbott Labs, Abbott Park IL), to name only a few.

In certain situations, the pharmaceutical compositions of the present disclosure can be delivered in a controlled release system. In one embodiment, a pump may be used (see Langer, supra; Sefton, 1987, CRC Crit. Ref. Biomed. Eng. 14:201). In another embodiment, polymeric materials can be used; see, Medical Applications of Controlled Release, Langer and Wise (eds.), 1974, CRC Pres., Boca Raton, Florida. In yet another embodiment, a controlled release system can be placed in proximity of the composition's target, thus requiring only a fraction of the systemic dose (see, e.g., Goodson, 1984, in Medical Applications of Controlled Release, supra, vol. 2, pp. 115-138). Other controlled release systems are discussed in the review by Langer, 1990, Science 249:1527-1533.

The injectable preparations may include dosage forms for intravenous, subcutaneous, intracutaneous and intramuscular injections, drip infusions, etc. These injectable preparations may be prepared by known methods. For example, the injectable preparations may be prepared, e.g., by dissolving, suspending or emulsifying the antibody or its salt described above in a sterile aqueous medium or an oily medium conventionally used for injections. As the aqueous medium for injections, there are, for example, physiological saline, an isotonic solution containing glucose and other auxiliary agents, etc., which may be used in combination with an appropriate solubilizing agent such as an alcohol (e.g., ethanol), a polyalcohol (e.g., propylene glycol, polyethylene glycol), a nonionic surfactant [e.g., polysorbate 80, HCO-50 (polyoxyethylene (50 mol) adduct of hydrogenated castor oil)], etc. As the oily medium, there are employed, e.g., sesame oil, soybean oil, etc., which may be used in combination with a solubilizing agent such as benzyl benzoate, benzyl alcohol, etc. The injection thus prepared is preferably filled in an appropriate ampoule.

Advantageously, the pharmaceutical compositions for oral or parenteral use described above are prepared into dosage forms in a unit dose suited to fit a dose of the active ingredients. Such dosage forms in a unit dose include, for example, tablets, pills, capsules, injections (ampoules), suppositories, etc.

### Dosage

The amount of active ingredient (e.g., GDF8 inhibitor, Activin A inhibitor, GLP-1 agonist) that can be administered to a subject is, generally, a therapeutically effective amount. As used herein, the phrase “therapeutically effective amount” means a dose of inhibitor, for example, antigen-specific binding protein and/or antigen-binding molecule, and/or of agonist that results in a detectable change in one or more of the following parameters: lean body mass (increase), fat mass (decrease), body weight (decrease), skeletal muscle mass (increase), plasma ALT and/or AST (decrease), liver triglyceride content/steatosis (decrease), and smooth-muscle actin in liver (decrease). In specific embodiments, the therapeutically effective amount of GDF8 inhibitor vs. Activin A inhibitor vs. GLP-1 agonist means the amount that achieves a distinct effect. For example, in one embodiment, the therapeutically effective amount of GLP-1 agonist is the amount that results in one or more of: (a) reduction of high sugar levels to normal levels; and/or (b) a detectable improvement in one or more symptoms or indicia of diabetes; and/or (c) an improvement in the liver dysfunction associated with NASH and/or treatment of NASH; and/or (d) an improvement in cholesterol, LDL cholesterol, and/or HDL cholesterol.

The dose of active ingredient (e.g., GDF8 inhibitor, Activin A inhibitor, GLP-1 agonist) may vary depending upon the age and the size of a subject to be administered, target disease, conditions, route of administration, and the like.

In the case of antibodies of the present disclosure (e.g., anti-GDF8 antibodies, anti-Activin A antibodies, anti-GLP-1 antibodies, anti-GLP-1R antibodies, or bispecific antibodies), a therapeutically effective amount can be from about 0.05 mg to about 600 mg; e.g., about 0.05 mg, about 0.1 mg, about 1.0 mg, about 1.5 mg, about 2.0 mg, about 10 mg, about 20 mg, about 30 mg, about 40 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 310 mg, about 320 mg, about 330 mg, about 340 mg, about 350 mg, about 360 mg, about 370 mg, about 380 mg, about 390 mg, about 400 mg, about 410 mg, about 420 mg, about 430 mg, about 440 mg, about 450 mg, about 460 mg, about 470 mg, about 480 mg, about 490 mg, about 500 mg, about 510 mg, about 520 mg, about 530 mg, about 540 mg, about 550 mg, about 560 mg, about 570 mg, about 580 mg, about 590 mg, or about 600 mg, of the respective antibody.

The amount of antibody of the present disclosure (e.g., anti-GDF8 antibodies, anti-Activin A antibodies, anti-GLP-1 antibodies, anti-GLP-1R antibodies, or bispecific antibodies) contained within the individual doses may be expressed in terms of milligrams of antibody per kilogram of patient body weight (i.e., mg/kg). For example, the anti-GDF8, anti-Activin A, anti-GDF8/anti-Activin A bispecific, and/or anti-GLP-1 antibodies of the present disclosure may be administered to a patient at a dose of about 0.0001 to about 50 mg/kg of patient body weight (e.g. 0.0001 mg/kg, 0.001 mg/kg, 0.01 mg/kg, 0.1 mg/kg, 0.5 mg/kg, 1.0 mg/kg, 1.5 mg/kg, 2.0 mg/kg, 2.5 mg/kg, 3.0 mg/kg, 3.5 mg/kg, 4.0 mg/kg, 4.5 mg/kg, 5.0 mg/kg, 5.5 mg/kg, 6.0 mg/kg, 6.5 mg/kg, 7.0 mg/kg, 7.5 mg/kg, 8.0 mg/kg, 8.5 mg/kg, 9.0 mg/kg, 9.5 mg/kg, 10.0 mg/kg, 10.5 mg/kg, 11.0 mg/kg, 11.5 mg/kg, etc.).

The GLP-1 agonists of the present disclosure may, in certain embodiments, be administered to a subject (patient) at a dose of about 0.05 mg/mL to about 5 mg/ml (e.g., about: 0.1 mg/mL, 0.2 mg/mL, 0.3 mg/mL, 0.4 mg/mL, 0.5 mg/mL, 0.6 mg/mL, 0.7 mg/mL, 0.8 mg/mL, 0.9 mg/mL, 1.0 mg/mL, 1.1 mg/mL, 1.2 mg/mL, 1.3 mg/mL, 1.4 mg/mL, 1.5 mg/mL, 1.6 mg/mL, 1.7 mg/mL, 2.0 mg/mL, 2.5 mg/mL, 3.0 mg/ml, 3.5 mg/mL, 4.0 mg/mL, 4.5 mg/mL, or 5 mg/mL).

The GLP-1 agonists of the present disclosure may, in certain embodiments, be administered to a subject (patient) at a dose of about 0.000001 mg/kg of body weight to about 50 mg/kg of body weight of the subject. In additional embodiments, the GLP-1 agonists (GLP-1R agonists) of the present disclosure are administered to a subject (patient) at a dose of about 0.00001 mg/kg of body weight to about 10 mg/kg of body weight of the subject. In still further embodiments, the GLP-1 agonists (GLP-1R agonists) of the present disclosure are administered to a subject (patient) at a dose of about 1 mg/kg of body weight of the subject. In still further embodiments, the GLP-1 agonists (GLP-1R agonists) of the present disclosure are administered to a subject (patient) at a dose of about 0.1 mg/kg of body weight of the subject. In still further embodiments, the GLP-1 agonists (GLP-1R agonists) of the present disclosure are administered to a subject (patient) at a dose of about 10 μg/kg of body weight of the subject. In certain embodiments, a GLP-1 receptor agonist of the present disclosure may be administered at one or more doses comprising between about 0.01 mg to about 60 mg. In additional embodiments, a GLP-1 receptor agonist of the present disclosure may be administered at one or more doses comprising between about 0.1 mg to about 6 mg.

In a specific embodiment, the anti-GDF8 antibody or antigen-binding fragment thereof is administered at a concentration of about 10 mg/kg to about 100 mg/kg. In another embodiment, the anti-GDF8 antibody or antigen-binding fragment thereof is administered at a concentration of about 50 mg/kg. In another specific embodiment, the anti-Activin A antibody or antigen-binding fragment thereof is administered at a concentration of about 10 mg/kg to about 100 mg/kg. In another embodiment, the anti-Activin A antibody or antigen-binding fragment thereof is administered at a concentration of about 50 mg/kg. In still another specific embodiment, the GLP-1 agonist is administered at a concentration of about 1 μg/kg to about 100 μg/kg. In yet another specific embodiment, the GLP-1 agonist is administered at a concentration of about 10 μg/kg. In a further embodiment, the GLP-1 agonist is administered at a concentration of about 0.833 mg/mL or about 7 μg/day (infusion). In still a further embodiment, the anti-GDF8 antibody or antigen-binding fragment thereof is administered at a concentration of about 50 mg/kg, the anti-Activin A antibody or antigen-binding fragment thereof is administered at a concentration of about 50 mg/kg, and the GLP-1 agonist is administered at a concentration of about 10 μg/kg.

The compositions of the present disclosure may comprise equal amounts of GDF8-specific binding protein and Activin A-specific binding protein. Alternatively, the amount of GDF8-specific binding protein in the composition may be less than or greater than the amount of Activin A-specific binding protein. Alternatively, the compositions of the present disclosure may comprise no Activin A-specific binding protein. A person of ordinary skill in the art, using routine experimentation and based on the instant disclosure, will be able to determine the appropriate amounts of the individual components in the compositions of the present disclosure necessary to produce a desired therapeutic effect.

### Therapeutic Methods

As used herein, the terms “treat”, “treating”, or “treatment” refer to the reduction or amelioration of the severity of at least one symptom or indication of a disease or disorder associated with GDF-8, Activin A, and/or GLP-1. In some embodiments, such a disease or disorder is obesity, diabetes, liver dysfunction, or some other condition associated with hyperglycemia. In one embodiment, the diabetes is Type 2 diabetes. The terms may also refer to inhibition of progression of disease or of worsening of symptoms. The terms may further refer to positive prognosis of disease, i.e., the subject may be free of a symptom or indication or may have reduced intensity of a symptom or indication upon administration of a therapeutic agent such as a composition (or compositions) according to the disclosure, i.e., a composition comprising a GDF8 inhibitor and a GLP-1 agonist, or a composition comprising a GDF8 inhibitor and an Activin A inhibitor and a GLP-1 agonist. The therapeutic agent may be administered at a therapeutic dose to the subject.

The terms “prevent”, “preventing” or “prevention” refer to inhibition of manifestation of any symptoms or indications of a disease or disorder associated with GDF-8, Activin A, and/or GLP-1. The terms may also refer to inhibition of manifestation of a symptom or indication of a disease or disorder associated with GDF-8, Activin A, and/or GLP-1 in a subject at risk for developing such a disease or disorder. In some embodiments, such a disease or disorder is obesity, diabetes, liver dysfunction, or some other condition associated with hyperglycemia. In one embodiment, the diabetes is Type 2 diabetes.

The present disclosure includes compositions and methods of treating conditions or afflictions which can be cured, alleviated or improved by increasing lean body mass and/or reducing fat mass in an individual, or by favorably altering glucose control, by specifically binding GDF8 and Activin A and exerting agonist activity on GLP-1. For example, the present disclosure includes compositions and methods for improving glucose control, increasing lean body mass, reducing fat mass, treating diabetes, and/or treating obesity in a subject, and/or treating liver issues associated with increased fat mass, obesity, and/or diabetes in a subject, the methods comprising, in certain embodiments, administering to the subject a composition comprising a GDF-8 inhibitor and a GLP-1 agonist, and in additional embodiments, administering to the subject a composition comprising a GDF-8 inhibitor, an Activin A inhibitor, and a GLP-1 agonist. The present disclosure also includes compositions and methods for improving glucose control, increasing lean body mass, reducing fat mass, treating diabetes, and/or treating obesity in a subject, and/or treating liver issues associated with increased fat mass, obesity, and/or diabetes in a subject, the methods comprising, in certain embodiments, administering to the subject a composition comprising a GDF-8 inhibitor and a GLP-1 agonist, and in additional embodiments, administering to the subject a composition comprising a GDF-8 inhibitor, an Activin A inhibitor, and a GLP-1 agonist, in a single composition or in more than one composition (for example, wherein each inhibitor and agonist is in a separate composition, or wherein the two inhibitors are in one composition, and the agonist is in another composition).

Treating liver issues may include lowering signs/symptoms of liver damage (for example, ALT/AST), lowering liver triglycerides, lowering steatosis, and/or lowering fibrosis (in the liver). In further embodiments, treating liver issues includes treating liver dysfunction, for example, hepatitis (A, B, C, D, and E), fatty liver disease (alcoholic and nonalcoholic), autoimmune disease (autoimmune hepatitis, primary biliary cirrhosis, primary sclerosing cholangitis), genetic conditions (hemochromatosis, Wilson's disease, alpha-1 antitrypsin deficiency), drug-induced liver disease, cancer (for example, hepatocellular carcinoma), cirrhosis, and liver failure. Any of the GDF-8 inhibitors (for example, GDF8-specific binding proteins) and Activin A inhibitors (for example, Activin A-specific binding proteins) and GLP-1 agonists (for example, GLP-1 receptor agonists) disclosed or referred to herein can be used in the context of these aspects of the disclosure. For example, the therapeutic methods of the present disclosure include administering to a subject an anti-GDF8 antibody and a GLP-1 agonist, or an anti-GDF8 antibody and an anti-Activin A antibody and a GLP-1 agonist.

The present disclosure also includes methods of managing or treating liver conditions or issues associated with elevated fat mass, obesity, and diabetes by administering a GDF8 inhibitor, and a GLP-1 agonist, or a GDF8 inhibitor, an Activin A inhibitor, and a GLP-1 agonist to a subject in need thereof. The management or treatment of liver conditions or issues is, in certain embodiments, in the form of decrease in plasma ALT and/or AST (markers of liver damage), decrease in liver triglyceride content/steatosis, and/or decrease in smooth-muscle active in liver (marker of fibrosis). Thus, in certain embodiments, administration of a composition according to the disclosure reduces liver damage or risk thereof, reduces steatosis or risk thereof, and/or reduces liver fibrosis or risk thereof in a subject.

In methods which comprise administering a GDF-8 inhibitor and a GLP-1 agonist, or a GDF-8 inhibitor and an Activin A inhibitor and a GLP-1 agonist, to a subject, the GDF-8 inhibitor and the Activin A inhibitor (if present) and the GLP-1 agonist may be administered to the subject at the same or substantially the same time, e.g., in a single therapeutic dosage, or in two or more separate dosages that are administered simultaneously or sequentially, e.g., in separate therapeutic dosages separated in time from one another, or in two or three separate doses that are administered simultaneously or sequentially.

The compositions of the present disclosure may be administered to a subject along with one or more additional therapeutic agents, including, e.g., growth factor inhibitors, immunosuppressants, anti-inflammatory agents, metabolic inhibitors, enzyme inhibitors, cytotoxic/cytostatic agents, and medicaments that control blood sugar levels (for example, metformin). The additional therapeutic agent may, in some embodiments, be selected from the group consisting of an insulin or insulin analogue, a biguanide (e.g., metformin), a thiazolidinedione, a sulfonylurea (e.g., chlorpropamide), a glinide (e.g., nateglinide), an alpha glucosidase inhibitor, a DPP4 inhibitor (e.g., sitagliptin), pramlintide, bromocriptine, a SGLT2 inhibitor (e.g., canagliflozin), an anti-hypertensive drug, a statin, aspirin, dietary modification, exercise, and a dietary supplement. The additional therapeutic agent(s) may be administered prior to, concurrent with, or after the administration of the GDF-8 inhibitor, the Activin A inhibitor (if present), and the GLP-1 agonist (or the composition(s) comprising the same) of the present disclosure.

Exemplary diseases, disorders and conditions that can be treated with the compositions of the present disclosure include, but are not limited to, sarcopenia, cachexia (either idiopathic or secondary to other conditions, e.g., cancer, chronic renal failure, or chronic obstructive pulmonary disease), muscle injury, muscle wasting and muscle atrophy, e.g., muscle atrophy or wasting caused by or associated with disuse, immobilization, bed rest, injury, medical treatment or surgical intervention (e.g., hip fracture, hip replacement, knee replacement, etc.) or by necessity of mechanical ventilation. The compositions of the disclosure may also be used to treat, prevent or ameliorate diseases such as cancer, obesity, diabetes, arthritis, multiple sclerosis, muscular dystrophy, amyotrophic lateral sclerosis, Parkinson's disease, osteoporosis, osteoarthritis, osteopenia, metabolic syndromes (including, but not limited to diabetes, obesity, nutritional disorders, organ atrophy, chronic obstructive pulmonary disease, and anorexia). The compositions of the disclosure may also be used to treat, prevent or ameliorate diseases such as diabetes mellitus, obesity, insulin resistance, hypertension, dyslipidemia, Type 2 diabetes, Type 1 diabetes, pre-diabetes, cardiovascular disease, atherosclerosis, congestive heart failure, coronary heart disease, arteriosclerosis, peripheral artery disease, stroke, respiratory dysfunction, renal disease, fatty liver disease, non-alcoholic steatohepatitis (NASH), and metabolic syndrome. NASH is a non-alcoholic fatty liver disease (NAFLD), characterized by steatosis of the liver accompanied by inflammation and hepatocyte ballooning, which can lead to advanced fibrosis, cirrhosis and hepatocellular carcinoma (Paternostro and Trauner, 2022, J Intern Med 0:1-15).

The compositions of the disclosure may also be used to treat, prevent or ameliorate diseases such as liver dysfunction, for example, hepatitis (A, B, C, D, and E), fatty liver disease (alcoholic and nonalcoholic), autoimmune disease (autoimmune hepatitis, primary biliary cirrhosis, primary sclerosing cholangitis), genetic conditions (hemochromatosis, Wilson's disease, alpha-1 antitrypsin deficiency), drug-induced liver disease, cancer (for example, hepatocellular carcinoma), cirrhosis, and liver failure.

### Administration Regimens

According to certain embodiments of the present disclosure, multiple doses of the compositions of the present disclosure (e.g., compositions comprising a GDF8 inhibitor and a GLP-1 agonist or compositions comprising a GDF8 inhibitor and an Activin A inhibitor and a GLP-1 agonist), may be administered to a subject over a defined time course. The methods according to this aspect of the disclosure comprise sequentially administering to a subject multiple doses of the compositions of the present disclosure. As used herein, “sequentially administering” means that each dose of the compositions of the present disclosure are administered to the subject at a different point in time, e.g., on different days separated by a predetermined interval (e.g., hours, days, weeks or months). The present disclosure includes methods which comprise sequentially administering to the patient an initial dose of a composition of the present disclosure, followed by one or more secondary doses of the composition, and optionally followed by one or more tertiary doses of the composition.

The terms “initial dose,” “secondary doses,” and “tertiary doses,” refer to the temporal sequence of administration of the compositions of the present disclosure. Thus, the “initial dose” is the dose which is administered at the beginning of the treatment regimen (also referred to as the “baseline dose”); the “secondary doses” are the doses which are administered after the initial dose; and the “tertiary doses” are the doses which are administered after the secondary doses. The initial, secondary, and tertiary doses may all contain the same amount of active ingredient(s), but will generally differ from one another in terms of frequency of administration. In certain embodiments, however, the amount of active ingredient(s) contained in the initial, secondary and/or tertiary doses will vary from one another (e.g., adjusted up or down as appropriate) during the course of treatment.

In one exemplary embodiment of the present disclosure, each secondary and/or tertiary dose is administered 1 to 30 (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or more) days after the immediately preceding dose. The phrase “the immediately preceding dose,” as used herein, means, in a sequence of multiple administrations, the dose(s) of the compositions of the present disclosure which are administered to a subject prior to the administration of the very next dose in the sequence with no intervening doses.

The methods according to this aspect of the disclosure may comprise administering to a patient any number of secondary and/or tertiary doses of the compositions of the present disclosure. For example, in certain embodiments, only a single secondary dose is administered to the patient. In other embodiments, two or more (e.g., 2, 3, 4, 5, 6, 7, 8, or more) secondary doses are administered to the patient. Likewise, in certain embodiments, only a single tertiary dose is administered to the patient. In other embodiments, two or more (e.g., 2, 3, 4, 5, 6, 7, 8, or more) tertiary doses are administered to the patient.

In embodiments involving multiple secondary doses, each secondary dose may be administered at the same frequency as the other secondary doses. For example, each secondary dose may be administered to the patient 1 to 29 days after the immediately preceding dose. Similarly, in embodiments involving multiple tertiary doses, each tertiary dose may be administered at the same frequency as the other tertiary doses. For example, each tertiary dose may be administered to the patient 1 to 60 days after the immediately preceding dose. Alternatively, the frequency at which the secondary and/or tertiary doses are administered to a patient can vary over the course of the treatment regimen. The frequency of administration may also be adjusted during the course of treatment by a physician depending on the needs of the individual patient following clinical examination.

### Sequences

The sequences referred to herein have SEQ ID NOs and sequences as shown in the following informal sequence table:

| | SEQ ID NO | amino acid sequence |
|---|---|---|
| GDF-8 (mature | 1 | Asp Phe Gly Leu Asp Cys Asp Glu His Ser Thr |
| protein) | | Glu Ser Arg Cys Cys Arg Tyr Pro Leu Thr Val Asp |
| | | Phe Glu Ala Phe Gly Trp Asp Trp Ile Ile Ala Pro |
| | | Lys Arg Tyr Lys Ala Asn Tyr Cys Ser Gly Glu Cys |
| | | Glu Phe Val Phe Leu Gln Lys Tyr Pro His Thr His |
| | | Leu Val His Gln Ala Asn Pro Arg Gly Ser Ala Gly |
| | | Pro Cys Cys Thr Pro Thr Lys Met Ser Pro Ile Asn |
| | | Met Leu Tyr Phe Asn Gly Lys Glu Gln Ile Ile Tyr |
| | | Gly Lys Ile Pro Ala Met Val Val Asp Arg Cys Gly |
| | | Cys Ser |
| βA subunit of Activin | 2 | Met Pro Leu Leu Trp Leu Arg Gly Phe Leu Leu |
| | | Ala Ser Cys Trp Ile Ile Val Arg Ser Ser Pro Thr |
| | | Pro Gly Ser Glu Gly His Ser Ala Ala Pro Asp Cys |
| | | Pro Ser Cys Ala Leu Ala Ala Leu Pro Lys Asp Val |
| | | Pro Asn Ser Gln Pro Glu Met Val Glu Ala Val Lys |
| | | Lys His Ile Leu Asn |
| | | Met Leu His Leu Lys Lys Arg Pro Asp Val Thr Gln |
| | | Pro Val Pro Lys Ala Ala Leu Leu Asn Ala Ile Arg |
| | | Lys Leu His Val Gly Lys Val Gly Glu Asn Gly Tyr |
| | | Val Glu Ile Glu Asp Asp Ile Gly Arg Arg Ala Glu |
| | | Met Asn Glu Leu Met Glu Gln Thr Ser Glu Ile Ile |
| | | Thr Phe Ala Glu Ser Gly Thr Ala Arg Lys Thr Leu |
| | | His Phe Glu Ile Ser Lys Glu Gly Ser Asp Leu Ser |
| | | Val Val Glu Arg Ala Glu Val Trp Leu Phe Leu Lys |
| | | Val Pro Lys Ala Asn Arg Thr Arg Thr Lys Val Thr |
| | | Ile Arg Leu Phe Gln Gln Gln Lys His Pro Gln Gly |
| | | Ser Leu Asp Thr Gly Glu Glu Ala Glu Glu Val Gly |
| | | Leu Lys Gly Glu Arg Ser Glu Leu Leu Leu Ser Glu |
| | | Lys Val Val Asp Ala Arg Lys Ser Thr Trp His Val |
| | | Phe Pro Val Ser Ser Ser Ile Gln Arg Leu Leu Asp |
| | | Gln Gly Lys Ser Ser Leu Asp Val Arg Ile Ala Cys |
| | | Glu Gln Cys Gln Glu Ser Gly Ala Ser Leu Val Leu |
| | | Leu Gly Lys Lys Lys Lys Lys Glu Glu Glu Gly Glu |
| | | Gly Lys Lys Lys |
| | | Gly Gly Gly Glu Gly Gly Ala Gly Ala Asp Glu Glu |
| | | Lys Glu Gln Ser His Arg Pro Phe Leu Met Leu |
| | | Gln Ala Arg Gln Ser Glu Asp His Pro His Arg Arg |
| | | Arg Arg Arg Gly Leu Glu Cys Asp Gly Lys Val Asn |
| | | Ile Cys Cys Lys Lys Gln Phe Phe Val Ser Phe Lys |
| | | Asp Ile Gly Trp Asn Asp Trp Ile Ile Ala Pro Ser |
| | | Gly Tyr His Ala Asn Tyr Cys Glu Gly Glu Cys Pro |
| | | Ser His Ile Ala Gly Thr Ser Gly Ser Ser Leu Ser |
| | | Phe His Ser Thr Val Ile Asn His Tyr Arg Met Arg |
| | | Gly His Ser Pro Phe Ala Asn Leu Lys Ser Cys |
| | | Cys Val Pro Thr Lys Leu Arg Pro Met Ser Met |
| | | Leu Tyr Tyr Asp Asp Gly Gln Asn Ile Ile Lys Lys |
| | | Asp Ile Gln Asn Met Ile Val Glu Glu Cys Gly Cys |
| | | Ser |
| βB subunit of Activin | 3 | Met Asp Gly Leu Pro Gly Arg Ala Leu Gly Ala Ala |
| | | Cys Leu Leu Leu Leu Ala Ala Gly Trp Leu Gly Pro |
| | | Glu Ala Trp Gly Ser Pro Thr Pro Pro Pro Thr Pro |
| | | Ala Ala Gln Pro Pro Pro Pro Pro Pro Gly Ser Pro |
| | | Gly Gly Ser Gln Asp Thr Cys Thr Ser Cys Gly Gly |
| | | Phe Arg Arg Pro Glu Glu Leu Gly Arg Val Asp Gly |
| | | Asp Phe Leu Glu Ala Val Lys Arg His Ile Leu Ser |
| | | Arg Leu Gln Met Arg Gly Arg Pro Asn Ile Thr His |
| | | Ala Val Pro Lys Ala Ala Met Val Thr Ala Leu Arg |
| | | Lys Leu His Ala Gly Lys Val Arg Glu Asp Gly Arg |
| | | Val Glu Ile Pro His Leu Asp Gly |
| | | His Ala Ser Pro Gly Ala Asp Gly Gln Glu Arg Val |
| | | Ser Glu Ile Ile Ser Phe Ala Glu Thr Asp Gly Leu |
| | | Ala Ser Ser Arg Val Arg Leu Tyr Phe Phe Ile Ser |
| | | Asn Glu Gly Asn Gln Asn Leu Phe Val Val Gln |
| | | Ala Ser Leu Trp Leu Tyr Leu Lys Leu Leu Pro Tyr |
| | | Val Leu Glu Lys Gly |
| | | Ser Arg Arg Lys Val Arg Val Lys Val Tyr Phe Gln |
| | | Glu Gln Gly His Gly Asp Arg Trp Asn Met Val Glu |
| | | Lys Arg Val Asp Leu Lys Arg Ser Gly Trp His Thr |
| | | Phe Pro Leu Thr Glu Ala Ile Gln Ala Leu Phe Glu |
| | | Arg Gly Glu Arg Arg Leu Asn Leu Asp Val Gln |
| | | Cys Asp Ser Cys Gln |
| | | Glu Leu Ala Val Val Pro Val Phe Val Asp Pro Gly |
| | | Glu Glu Ser His Arg Pro Phe Val Val Val Gln Ala |
| | | Arg Leu Gly Asp Ser Arg His Arg Ile Arg Lys Arg |
| | | Gly Leu Glu Cys Asp Gly Arg Thr Asn Leu Cys |
| | | Cys Arg Gln Gln Phe Phe Ile Asp Phe Arg Leu Ile |
| | | Gly Trp Asn Asp Trp |
| | | Ile Ile Ala Pro Thr Ser Tyr Tyr Gly Asn Tyr Cys |
| | | Glu Gly Ser Cys Pro Ala Tyr Leu Ala Gly Val Pro |
| | | Gly Ser Ala Ser Ser Phe His Thr Ala Val Val Asn |
| | | Gln Tyr Arg Met Arg Gly Leu Asn Pro Gly Thr Val |
| | | Asn Ser Cys Cys Ile Pro Thr Lys Leu Ser Thr Met |
| | | Ser Met Leu Tyr |
| | | Phe Asp Asp Glu Tyr Asn Ile Val Lys Arg Asp Val |
| | | Pro Asn Met Ile Val Glu Glu Cys Gly Cys Ala Ser |
| | | Pro Gly Lys |
| anti-GDF8 ab HCVR | 4 | Glu Val Gln Val Leu Glu Ser Gly Gly Asp Leu Val Gln |
| | | Pro Gly Gly Ser Leu Arg Leu Ser Cys Ala Ala Ser Gly |
| | | Phe Thr Phe Ser Ala Tyr Ala Met Thr Trp Val Arg Gln |
| | | Ala Pro Gly Lys Gly Leu Glu Trp Val Ser Ala Ile Ser |
| | | Gly Ser Gly Gly Ser Ala Tyr Tyr Ala Asp Ser Val Lys |
| | | Gly Arg Phe Thr Ile Ser Arg Asp Asn Ser Lys Asn Thr |
| | | Val Tyr Leu Gln Met Asn Ser Leu Arg Ala Glu Asp Thr |
| | | Ala Val Tyr Tyr Cys Ala Lys Asp Gly Ala Trp Lys Met |
| | | Ser Gly Leu Asp Val Trp Gly Gln Gly Thr Thr Val Ile |
| | | Val Ser Ser |
| anti-GDF8 ab LCVR | 5 | Asp Ile Gln Met Thr Gln Ser Pro Ala Ser Leu Ser Ala |
| | | Ser Val Gly Asp Arg Val Thr Ile Thr Cys Arg Ala Ser |
| | | Gln Asp Ile Ser Asp Tyr Leu Ala Trp Tyr Gln Gln Lys |
| | | Pro Gly Lys Ile Pro Arg Leu Leu Ile Tyr Thr Thr Ser |
| | | Thr Leu Gln Ser Gly Val Pro Ser Arg Phe Arg Gly Ser |
| | | Gly Ser Gly Thr Asp Phe Thr Leu Thr Ile Ser Ser Leu |
| | | Gln Pro Glu Asp Val Ala Thr Tyr Tyr Cys Gln Lys Tyr |
| | | Asp Ser Ala Pro Leu Thr Phe Gly Gly Gly Thr Lys Val |
| | | Glu Ile Lys |
| anti-GDF8 ab HCDR1 | 6 | Gly Phe Thr Phe Ser Ala Tyr Ala |
| anti-GDF8 ab HCDR2 | 7 | Ile Ser Gly Ser Gly Gly Ser Ala |
| anti-GDF8 ab HCDR3 | 8 | Ala Lys Asp Gly Ala Trp Lys Met Ser Gly Leu Asp Val |
| anti-GDF8 ab LCDR1 | 9 | Gln Asp Ile Ser Asp Tyr |
| anti-GDF8 ab LCDR2 | | Thr Thr Ser |
| anti-GDF8 ab LCDR3 | 11 | Gln Lys Tyr Asp Ser Ala Pro Leu Thr |
| anti-Activin A ab | 12 | Gln Val Gln Leu Gln Glu Ser Gly Pro Gly Leu Val Lys |
| REGN2477* HCVR | | Pro Ser Glu Thr Leu Ser Leu Thr Cys Thr Val Ser Gly |
| | | Gly Ser Phe Ser Ser His Phe Trp Ser Trp Ile Arg Gln |
| | | Pro Pro Gly Lys Gly Leu Glu Trp Ile Gly Tyr Ile Leu |
| | | Tyr Thr Gly Gly Thr Ser Phe Asn Pro Ser Leu Lys Ser |
| | | Arg Val Ser Met Ser Val Gly Thr Ser Lys Asn Gln Phe |
| | | Ser Leu Lys Leu Ser Ser Val Thr Ala Ala Asp Thr Ala |
| | | Val Tyr Tyr Cys Ala Arg Ala Arg Ser Gly Ile Thr Phe |
| | | Thr Gly Ile Ile Val Pro Gly Ser Phe Asp Ile Trp Gly Gln |
| | | Gly Thr Met Val Thr Val Ser Ser |
| anti-Activin A ab | 13 | Glu Ile Val Leu Thr Gln Ser Pro Gly Thr Leu Ser Leu |
| REGN2477 LCVR | | Ser Pro Gly Glu Arg Ala Thr Leu Ser Cys Arg Ala Ser |
| | | Gln Ser Val Ser Ser Ser Tyr Leu Ala Trp Tyr Gln Gln |
| | | Lys Pro Gly Gln Ala Pro Arg Leu Leu Ile Tyr Gly Ala |
| | | Ser Ser Arg Ala Thr Gly Ile Pro Asp Arg Phe Ser Gly |
| | | Ser Gly Ser Gly Thr Asp Phe Thr Leu Thr Ile Ser Arg |
| | | Leu Glu Pro Glu Asp Phe Ala Val Tyr Tyr Cys Gln Gln |
| | | Tyr Gly Ser Ser Pro Trp Thr Phe Gly Gln Gly Thr Lys |
| | | Val Glu Ile Lys |
| anti-Activin A ab | 14 | Gly Gly Ser Phe Ser Ser His Phe |
| REGN2477 HCDR1 | | |
| anti-Activin A ab | 15 | Ile Leu Tyr Thr Gly Gly Thr |
| REGN2477 HCDR2 | | |
| anti-Activin A ab | 16 | Ala Arg Ala Arg Ser Gly Ile Thr Phe Thr Gly Ile Ile Val |
| REGN2477 HCDR3 | | Pro Gly Ser Phe Asp Ile |
| anti-Activin A ab | 17 | Gln Ser Val Ser Ser Ser Tyr |
| REGN2477 LCDR1 | | |
| anti-Activin A ab | | Gly Ala Ser |
| REGN2477 LCDR2 | | |
| anti-Activin A ab | 19 | Gln Gln Tyr Gly Ser Ser Pro Trp Thr |
| REGN2477 LCDR3 | | |
| anti-Activin A ab | 21 | |
| REGN2376** HCVR | | |
| anti-Activin A ab | 22 | |
| REGN2376 LCVR | | |
| anti-Activin A ab | 23 | |
| REGN2376 HCDR1 | | |
| anti-Activin A ab | 24 | |
| REGN2376 HCDR2 | | |
| anti-Activin A ab | 25 | |
| REGN2376 HCDR3 | | |
| anti-Activin A ab | 26 | |
| REGN2376 LCDR1 | | |
| anti-Activin A ab | | Asp Ala Ser |
| REGN2376 LCDR2 | | |
| anti-Activin A ab | 28 | |
| REGN2376 LCDR3 | | |

*= H4H10446P2;
**= H4H10430P

### EXAMPLES

The following examples are put forth so as to provide those of ordinary skill in the art with a complete disclosure and description of how to make and use the methods and compositions of the disclosure, and are not intended to limit the scope of what the inventors regard as their disclosure. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

### Example 1: In Vivo Efficacy Study to Examine the Effect of REGN1033 & REGN2477 on Body Composition and Glucose Metabolism Alone and in Combination With the GLP-1 Agonist Semaglutide

In an effort to study the effects of GDF8 and Activin A inhibition with and without GLP-1 agonism on body composition, a treatment study using a diet-induced obesity (DIO) mouse model was performed. DIO mice that have been fed a high fat diet for at least 20 weeks were distributed into 4 different groups balanced by body fat content as determined by MRI and blood glucose. On day 0, all mice were implanted with osmotic pumps (Alzet, Cat #2004), in which the stainless steel flow moderators were replaced with PEEK tubing (DURECT Corporation, Cat #0002496) to allow for MRI measurements during the experiment. Pumps for groups 1 and 2 contained PBS, while pumps used for groups 3 and 4 contained Semaglutide (0.833 mg/mL for 7 ug/day infusion). The next day (day 1), mice were injected with their respective antibodies: group 1 and group 3: Isotype control (at 20 mg/kg); group 2 and group 4: anti-GDF8 antibody (REGN1033) & anti-Activin A antibody (REGN2477) (at 10 mg/kg each). The antibody injections were repeated on days 4 and 7 and once weekly after that.

Body composition using MRI and fed blood glucose using a glucometer from tail bleeds were measured weekly in the morning. Mice were also bled for insulin measurements on a weekly basis. An oral glucose tolerance test (OGTT) was performed on day 23. For the OGTT, mice were fasted for 4 hours, and baseline glucose was measured. Subsequently, 2 g/kg glucose was administered to the mice by oral gavage, and glucose was measured again after 30, 60, 90, and 120 minutes. On day 26, 6 hour fasted blood glucose was measured, and mice were bled for insulin assessment as described above. At the end of the study (day 28/29), mice were sacrificed, and tissue (skeletal muscle, adipose, pancreas, heart, spleen, liver) was collected for weight and further analysis. Mouse plasma was used to measure circulating levels of ALT, AST, non-esterified fatty acids (NEFAs), triglycerides (trigs), and cholesterol (chol) on the Advia Chemistry Analyzer from Siemens. One lobe of the liver was fresh frozen to allow for triglyceride extraction and quantification using standard protocols. Another lobe of the liver and the pancreas were fixed in 10% formalin, embedded in paraffin, and sectioned for liver histology. H&E, as well as smooth muscle actin (SMA), staining was performed, and slides were subjected to quantification using Halo software. Paraffin sections of the pancreas were stained for insulin and glucagon to determine beta cell and alpha cell mass, respectively, using Halo software.

### Results

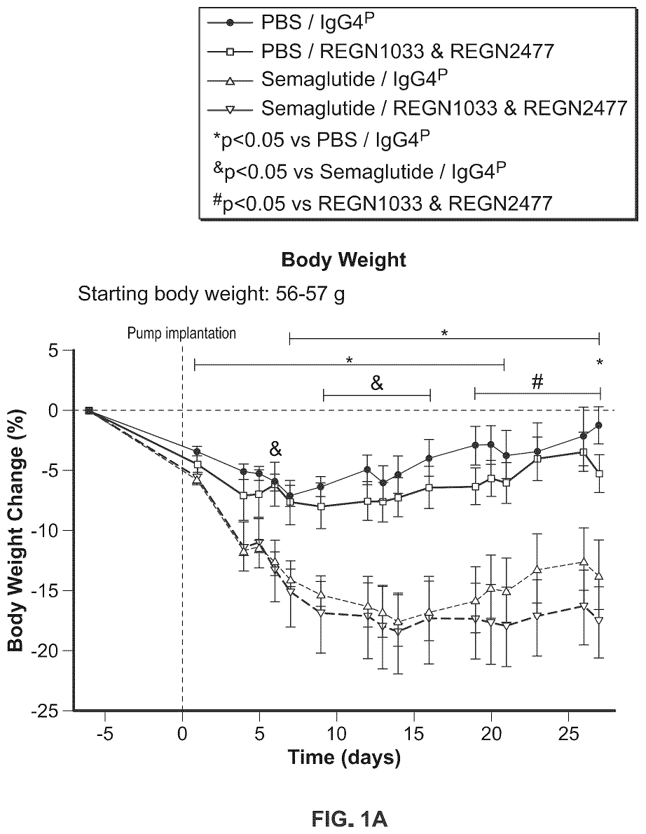

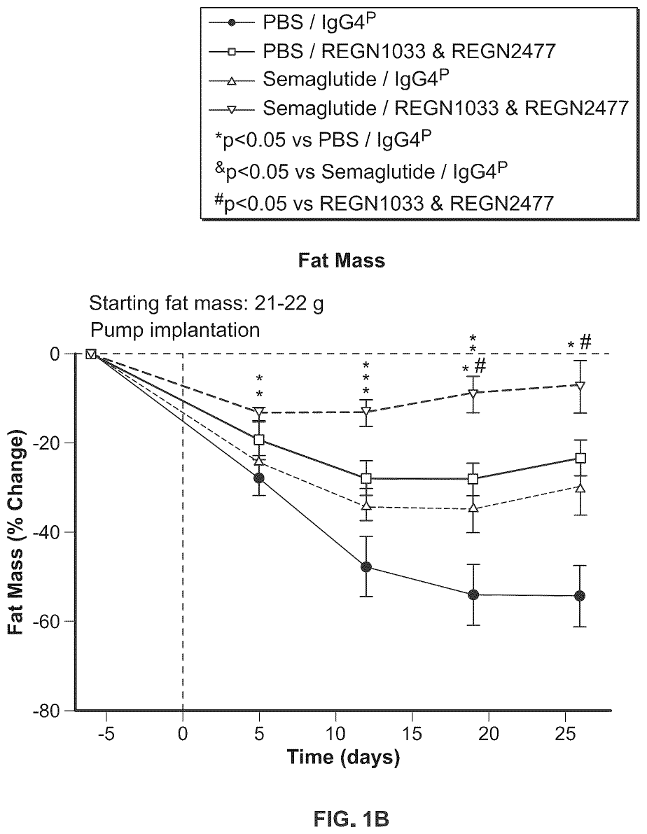

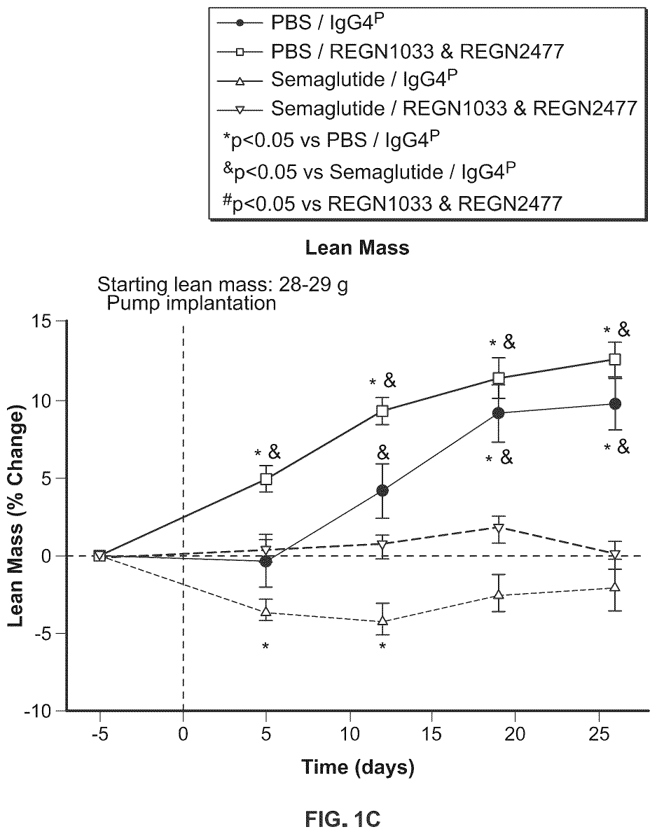

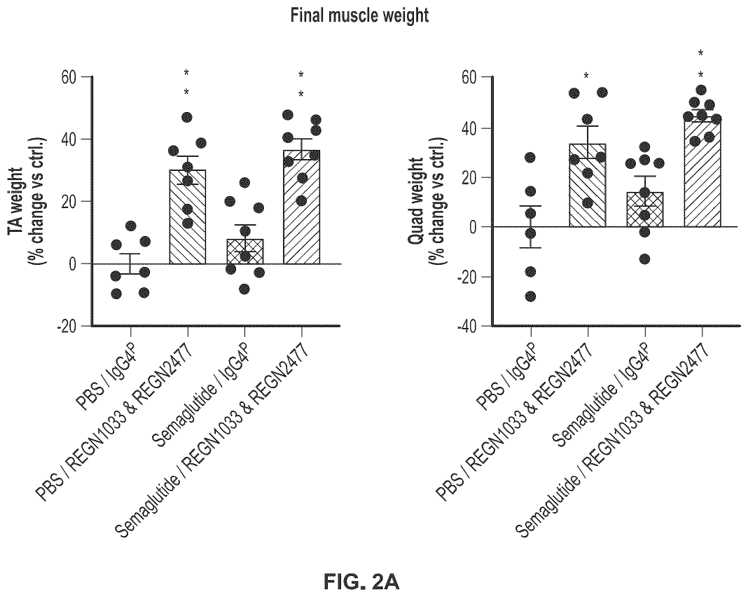

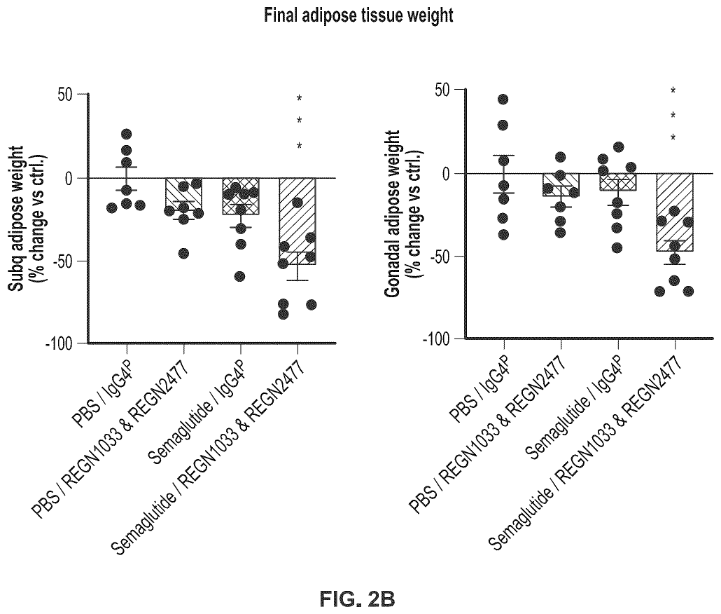

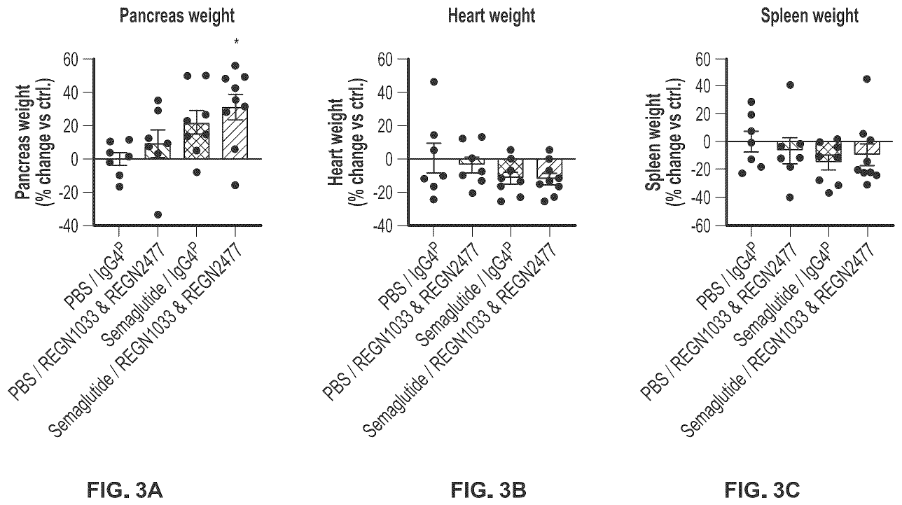

Treatment with REGN1033 & REGN2477 in DIO mice resulted in about 10% increase in lean mass and 15% loss in fat mass compared to the control group (Group 1). Overall, body weight was not changed in the isotype group. Semaglutide treatment decreased body weight (approximately 14% as compared with the control group), which was associated with a decrease in fat mass (approximately 18% as compared with the control group) and a smaller decrease in lean mass (approximately 3-4% as compared with the control group) (FIGS. 1A-1C). The combination of REGN1033 & REGN2477 and Semaglutide (group 4) seemed to have additive effects on fat loss (approximately 50% as compared with the control group), while maintaining the increase in lean mass (8% as compared with the control group) observed with REGN1033 and REGN2477 alone. Because of the increase in lean mass, body weight loss of group 4 was similar to what was observed in mice treated with Semaglutide alone (group 3). The increase in skeletal muscle weight (approximately 40% as compared with the control group) at the end of the study confirmed that the larger lean mass mediated by REGN1033 & REGN2477 was a result of an increase in muscle mass, and not heart or spleen mass (FIG. 2A). In addition, there was a significant decrease in subcutaneous and gonadal fat pads (50% as compared with the control group) only in group 4 when REGN1033 & REGN2477 was combined with Semaglutide treatment (FIG. 2B).

TABLE 1
Body weight changes over time

| | | | | PBS/REGN1033 & | | | Semaglutide/ | | | Semaglutide/REGN1033 & | | |
|---|---|---|---|---|---|---|---|---|---|---|---|---|
| Group | PBS/IgG4^{P} | | | REGN2477 | | | IgG4^{P} | | | REGN2477 | | |
| Day | Mean | Sem | N | Mean | Sem | N | Mean | Sem | N | Mean | Sem | N |
| −6 | 0 | 0 | 5 | 0 | 0 | 7 | 0 | 0 | 8 | 0 | 0 | [symbol] |
| 1 | −3.42 | 0.37 | 8 | −4.47 | [symbol] | 7 | [symbol] | 0.25 | [symbol] | −5.73 | 0.57 | 8 |
| 4 | [symbol] | [symbol] | [symbol] | [symbol] | 2.05 | 7 | [symbol] | 0.45 | [symbol] | −11.32 | 2.05 | [symbol] |
| 5 | −5.28 | [symbol] | [symbol] | −6.94 | 1.95 | 7 | −11.33 | [symbol] | 8 | −11.09 | 2.04 | [symbol] |
| 6 | −6.90 | [symbol] | 8 | [symbol] | 1.79 | 7 | −12.46 | 0.74 | [symbol] | [symbol] | [symbol] | 8 |
| 7 | −7.08 | 0.53 | 5 | −7.64 | 1.86 | 7 | [symbol] | 0.75 | 8 | −15.28 | 2.74 | 5 |
| 9 | −6.35 | 0.82 | 8 | −7.97 | [symbol] | 7 | −15.25 | 1.03 | 5 | −16.97 | [symbol] | 8 |
| 12 | −4.92 | 1.23 | 8 | −7.55 | [symbol] | 7 | −16.20 | 1.88 | [symbol] | −17.28 | 3.40 | 8 |
| 13 | −5.99 | 1.42 | 5 | −7.60 | [symbol] | 7 | −15.72 | 2.15 | 8 | [symbol] | 3.45 | 5 |
| 14 | [symbol] | 1.61 | 8 | −7.23 | 1.55 | 7 | −17.49 | 2.12 | 5 | [symbol] | [symbol] | 8 |
| [symbol] | −3.98 | 1.54 | [symbol] | [symbol] | 1.74 | 7 | −16.71 | [symbol] | 8 | [symbol] | 3.64 | [symbol] |
| 19 | −2.88 | 1.61 | [symbol] | −6.31 | [symbol] | 7 | −15.74 | 2.74 | 8 | −17.50 | 3.14 | [symbol] |
| 20 | −2.86 | [symbol] | 8 | [symbol] | 1.52 | 7 | [symbol] | 2.66 | 5 | −17.81 | [symbol] | 8 |
| 21 | −3.76 | [symbol] | 5 | [symbol] | 1.89 | 7 | [symbol] | [symbol] | 8 | −18.05 | [symbol] | [symbol] |
| 23 | −3.42 | 2.40 | 8 | −4.04 | 1.78 | 7 | −13.16 | 2.90 | 8 | −17.26 | 3.20 | 8 |
| 26 | −2.14 | 2.43 | 8 | −3.48 | [symbol] | 7 | −12.53 | 2.80 | [symbol] | −18.43 | 3.10 | 8 |
| 27 | [symbol] | 1.51 | 7 | [symbol] | 1.61 | 7 | [symbol] | 2.59 | 8 | −17.54 | 2.99 | 5 |

[symbol]

indicates data missing or illegible when filed

TABLE 2
Lean mass changes over time

| | | | | PBS/REGN1033 & | | | Semaglutide/ | | | Semaglutide/REGN1033 & | | |
|---|---|---|---|---|---|---|---|---|---|---|---|---|
| Group | PBS/IgG4^{P} | | | REGN2477 | | | IgG4^{P} | | | REGN2477 | | |
| Day | Mean | Sem | N | Mean | Sem | N | Mean | Sem | N | Mean | Sem | N |
| −6 | 0 | 0 | 5 | 0 | 0 | 7 | 0 | 0 | 8 | 0 | 0 | 8 |
| 5 | 0.27 | 0.72 | 8 | 4.92 | 0.85 | 7 | −3.50 | 0.70 | 8 | −0.36 | 1.70 | 8 |
| 12 | 0.56 | 0.75 | 8 | 9.28 | 0.88 | 7 | −4.09 | 1.01 | 5 | 4.15 | 1.74 | 5 |
| 19 | [symbol] | 0.87 | 5 | 11.40 | 1.30 | 7 | −2.43 | 1.15 | 8 | [symbol] | 1.84 | 8 |
| 25 | −0.03 | 0.92 | 8 | 12.58 | [symbol] | 7 | −1.93 | [symbol] | 8 | 9.75 | 1.63 | [symbol] |

[symbol]

indicates data missing or illegible when filed

TABLE 3
Fat mass changes over time

| | | | | PBS/REGN1033 & | | | Semaglutide/ | | | Semaglutide/REGN1033 & | | |
|---|---|---|---|---|---|---|---|---|---|---|---|---|
| Group | PBS/IgG4^{P} | | | REGN2477 | | | IgG4^{P} | | | REGN2477 | | |
| Day | Mean | Sem | N | Mean | Sem | N | Mean | Sem | N | Mean | Sem | N |
| −6 | 0 | 0 | 8 | 0 | 0 | 7 | 0 | 0 | 8 | 0 | 0 | 8 |
| 5 | [symbol] | 1.46 | 8 | −19.18 | [symbol] | 7 | −23.75 | 0.83 | 8 | −27.70 | 3.98 | 8 |
| 12 | −13.12 | 2.82 | 8 | −27.93 | [symbol] | 7 | −33.78 | 3.65 | 8 | −47.77 | [symbol] | [symbol] |
| 19 | [symbol] | [symbol] | 8 | [symbol] | 3.53 | 7 | −34.24 | 5.83 | 8 | −53.98 | 8.77 | 8 |
| 26 | −7.36 | [symbol] | 8 | −23.39 | 4.01 | 7 | −29.52 | [symbol] | 8 | −54.22 | [symbol] | [symbol] |

[symbol]

indicates data missing or illegible when filed

TABLE 4
Final organ weights

| | | | | PBS/REGN1033 & | | | Semaglutide/ | | | Semaglutide/REGN1033 & | | |
|---|---|---|---|---|---|---|---|---|---|---|---|---|
| Group | PBS/IgG4^{P} | | | REGN2477 | | | IgG4^{P} | | | REGN2477 | | |
| Tissue | Mean | Sem | N | Mean | Sem | N | Mean | Sem | N | Mean | Sem | N |
| Tbialis anterior | 0.00 | 3.229 | 7 | 30.20 | [symbol] | 7 | [symbol] | [symbol] | 8 | [symbol] | 3.40 | 5 |
| Gastroc [symbol] mius | 0.00 | 8.40 | 8 | 34.05 | [symbol] | 7 | [symbol] | 5.72 | 8 | 44.82 | 2.44 | 8 |
| Subq Fat | 0.00 | [symbol] | 7 | [symbol] | [symbol] | 7 | −22.58 | [symbol] | 5 | −53.05 | 8.35 | 8 |
| Gonadal Fat | 0.00 | 11.28 | 7 | [symbol] | 5.96 | 7 | −10.56 | [symbol] | 8 | −41.33 | 9.55 | 5 |
| Pancre [symbol] | 0.00 | 3.88 | 7 | [symbol] | 8.34 | 7 | 22.02 | 7.19 | 8 | 31.90 | [symbol] | 8 |
| Heart | 0.00 | 9.06 | 7 | −4.14 | 4.52 | 7 | [symbol] | [symbol] | 5 | [symbol] | 3.69 | 8 |
| Spleen | 0.00 | 7.35 | 7 | [symbol] | 9.24 | 7 | [symbol] | 5.25 | 8 | [symbol] | [symbol] | 5 |

[symbol]

indicates data missing or illegible when filed

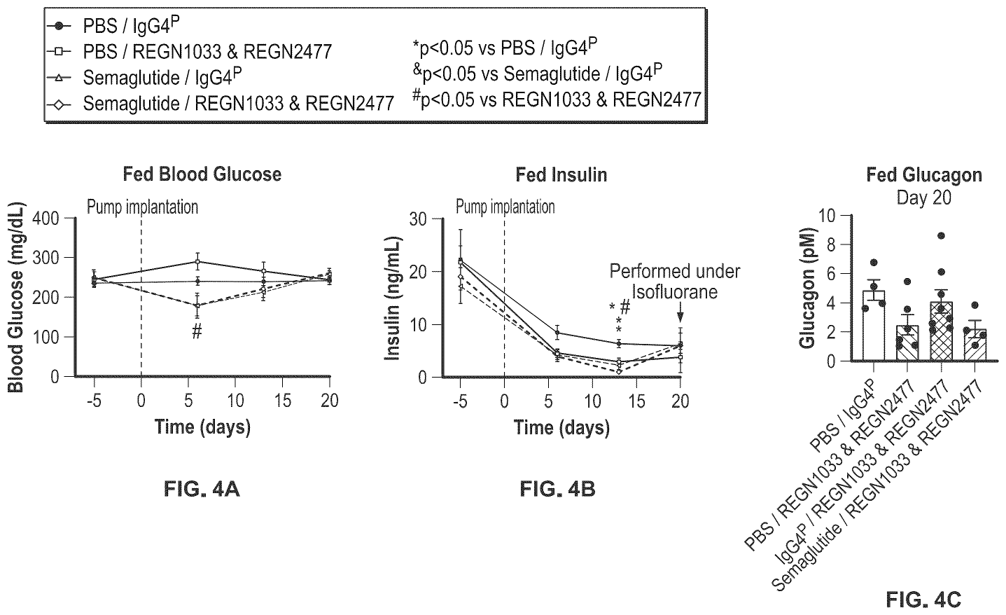

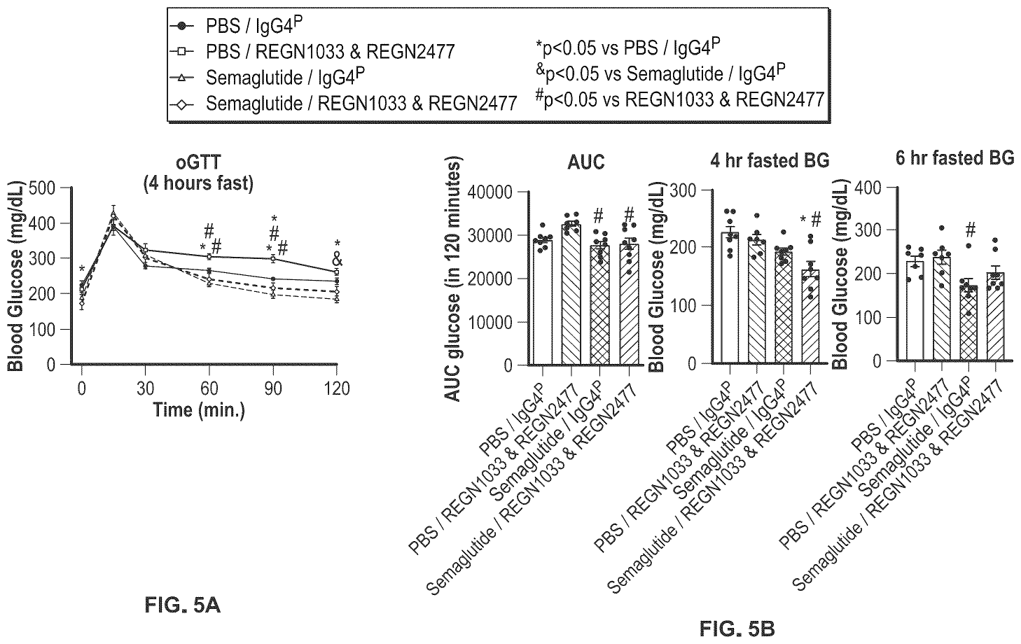

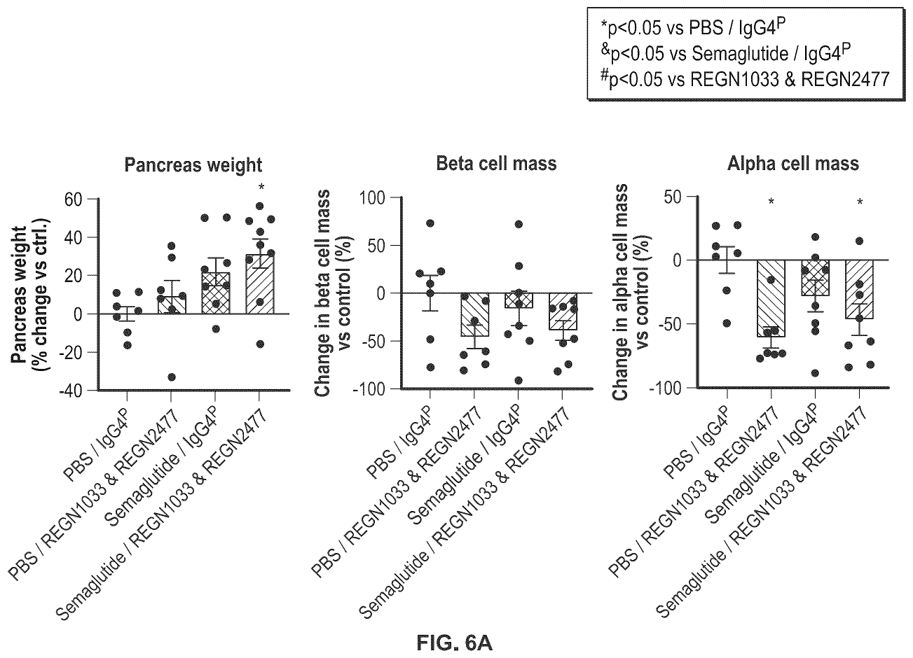

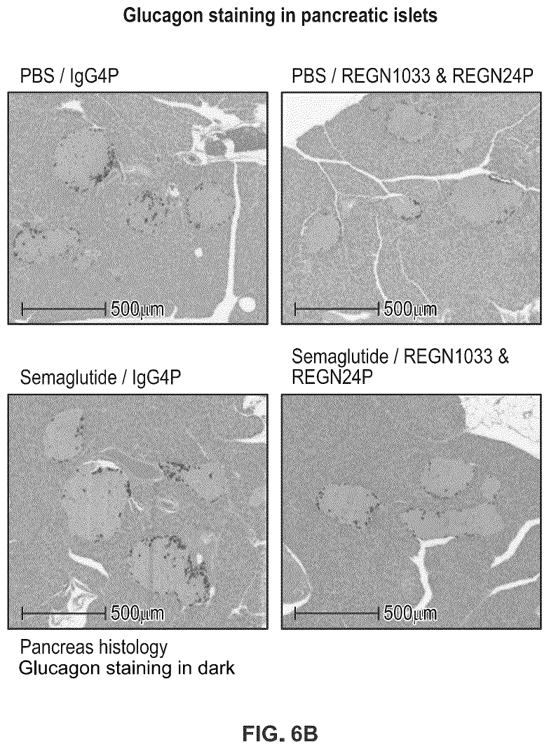

Throughout the study, no major changes in glucose metabolism were observed. All Semaglutide-treated mice (group 3 and 4) showed a temporal decrease in fed glucose at day 7, but this was no longer observed at later days (FIG. 4A). While fasting glucose was lower in Semaglutide-treated groups at later times during the study, there was no major change observed in glucose tolerance. However, a decrease in circulating insulin levels in all treatment groups compared to the control groups was observed, indicating that the glucose could be controlled with less insulin (FIG. 4B). In addition, glucagon levels appear to be lower in REGN1033 & REGN2477-treated groups (FIG. 4C). Pancreas mass was increased significantly in this study when REGN1033 & REGN2477 was combined with Semaglutide treatment (FIG. 6A). This effect is believed to be driven by Semaglutide and has been reported before with other GLP-1 agonist or compounds activating this pathway (PMID: 19509017, 22266668). To examine whether changes in pancreas mass, insulin, and glucagon were driven by changes in beta or alpha cell mass, pancreas histology was performed (FIG. 6B). While there was no significant change in beta cell mass, a trend was observed towards lower beta cell mass in groups 2 and 4 when REGN1033 & REGN2477 was given. In addition, there was a significant drop in alpha cell mass (approximately 50% as compared with the control group) in mice treated with REGN1033 & REGN2477, which could explain the reduction in glucagon seen above.

TABLE 5
Fed glucose over time

| | | | | PBS/REGN1033 & | | | Semaglutide/ | | | Semaglutide/REGN1033 & | | |
|---|---|---|---|---|---|---|---|---|---|---|---|---|
| Group | PBS/IgG4^{P} | | | REGN2477 | | | IgG4^{P} | | | REGN2477 | | |
| Day | Mean | SEM | N | Mean | SEM | N | Mean | SEM | N | Mean | SEM | N |
| −5 | 235.25 | 9.336468 | 8 | 243.7143 | [symbol] | 7 | [symbol] | [symbol] | 8 | [symbol] | [symbol] | [symbol] |
| 6 | [symbol] | 12.93864 | 8 | 290 | 21.95016 | 7 | [symbol] | 26.50522 | 8 | [symbol] | [symbol] | 8 |
| 13 | [symbol] | [symbol] | 8 | [symbol] | [symbol] | 7 | [symbol] | 22.26143 | 8 | 221.575 | 22.4136737 | 5 |
| 20 | 239.5 | [symbol] | 8 | [symbol] | 10.32993 | 7 | 259.5 | [symbol] | 8 | 259.78 | [symbol] | 8 |

[symbol]

indicates data missing or illegible when filed

TABLE 6
fed insulin over time

| | | | | PBS/REGN1033 & | | | Semaglutide/ | | | Semaglutide/REGN1033 & | | |
|---|---|---|---|---|---|---|---|---|---|---|---|---|
| Group | PBS/IgG4^{P} | | | REGN2477 | | | IgG4^{P} | | | REGN2477 | | |
| Day | Mean | SEM | N | | SEM | N | Meat | SEM | N | Mean | SEM | N |
| −5 | [symbol] | [symbol] | 5 | 21.78 | [symbol] | 7 | 16.285 | 2.462359 | [symbol] | [symbol] | 2.23657618 | 8 |
| 6 | [symbol] | 1.047137 | 8 | 6.221429 | 0.539856 | 7 | 6.2175 | [symbol] | 8 | 3.77625 | 0.83936825 | 8 |
| 13 | 3.42125 | 1.174269 | 5 | [symbol] | [symbol] | 7 | 2.29625 | [symbol] | [symbol] | [symbol] | [symbol] | 8 |
| 20 | 5.92 | 2.521493 | 8 | [symbol] | 3.004551 | 7 | [symbol] | 3.072524 | 8 | 6.0625 | 3.34560092 | 8 |
| 26 | 11.14 | 2.92048 | 7 | 4.814286 | 0.547091 | 7 | [symbol] | [symbol] | [symbol] | 5.43 | 3.99244376 | 8 |

[symbol]

indicates data missing or illegible when filed

TABLE 7
Fed glucagon levels at day 20

| | | | PBS/REGN1033 & | | | Semaglutide/ | | | Semaglutide/REGN1033 & | | |
|---|---|---|---|---|---|---|---|---|---|---|---|
| PBS/IgG4^{P} | | | REGN2477 | | | IgG4^{P} | | | REGN2477 | | |
| Mean | SEM | N | Mean | SEM | N | Mean | SEM | N | Mean | SEM | N |
| 4.871636 | 0.713498 | 4 | 2.480572 | 0.711113 | 6 | 4.105623 | 0.798253 | 8 | 2.205669 | 0.58101953 | 4 |

TABLE 8
6 hrs fasted glucose levels at day 26

| | | PBS/REGN1033 & | | Semaglutide/REGN1033 |
|---|---|---|---|---|
| | PBS/IgG4^{P} | REGN2477 | Semaglutide/IgG4^{P} | & REGN2477 |
| N | 7 | 7 | 8 | 8 |
| Mean | 228 | 237 | 172.6 | 201.5 |
| Std. Deviation | 30.2 | 41.97 | 43.05 | 41.55 |
| Std. Error of Mean | 11.42 | 15.86 | 15.22 | 14.69 |

TABLE 9
4 hrs fasted glucose levels at day 23

| | | PBS/REGN1033 & | | Semaglutide/REGN1033 |
|---|---|---|---|---|
| | PBS/IgG4^{P} | REGN2477 | Semaglutide/IgG4^{P} | & REGN2477 |
| N | 6 | 7 | 8 | 8 |
| Mean | 225 | 212.6 | 192.1 | 161.9 |
| Std. Deviation | 28.7 | 23.51 | 17.11 | 36.73 |
| Std. Error of Mean | 10.15 | 8.888 | 6.049 | 12.99 |

TABLE 10
Area under the curve of OGTT at day 23

| | | PBS/REGN1033 & | | Semaglutide/REGN1033 |
|---|---|---|---|---|
| | PBS/IgG4^{P} | REGN2477 | Semaglutide/IgG4^{P} | & REGN2477 |
| N | 8 | 7 | 8 | 9 |
| Mean | 28997 | 32633 | 27698 | 27963 |
| Std. Deviation | 1791 | 1679 | 2489 | 3809 |
| Std. Error of Mean | 633.1 | 634.6 | 879.9 | 1270 |

TABLE 11
oGTT measurement on day 23

| | | | | PBS/REGN1033 & | | | Semaglutide/ | | | Semaglutide/REGN1033 & | | |
|---|---|---|---|---|---|---|---|---|---|---|---|---|
| Group | PBS/IgG4^{P} | | | REGN2477 | | | IgG4^{P} | | | REGN2477 | | |
| Time (min) | Mean | SEM | N | Mean | SEM | N | Mean | SEM | N | Mean | SEM | N |
| 0 | 225 | 10.14713 | 8 | 212.5714 | [symbol] | 7 | 192.125 | [symbol] | 8 | [symbol] | [symbol] | 9 |
| 15 | 388.25 | [symbol] | 8 | 392.8571 | 15.00385 | 7 | 433.5 | 17.34317 | 8 | 422.333333 | [symbol] | 9 |
| 30 | [symbol] | [symbol] | 8 | 323.7143 | [symbol] | 7 | 310.75 | [symbol] | 5 | [symbol] | [symbol] | 9 |
| 60 | 266.25 | [symbol] | 8 | 305.2857 | [symbol] | 7 | 230.75 | 11.50582 | 8 | 239.444444 | [symbol] | 9 |
| 90 | 242.375 | [symbol] | 8 | [symbol] | [symbol] | 7 | [symbol] | [symbol] | 8 | 215 | [symbol] | 9 |
| 120 | 235.25 | [symbol] | 8 | [symbol] | 9.457747 | 7 | [symbol] | [symbol] | 5 | 204.111111 | [symbol] | 9 |

[symbol]

indicates data missing or illegible when filed

TABLE 12
Change in pancreas weight

| | | PBS/REGN1033 & | | Semaglutide/REGN1033 |
|---|---|---|---|---|
| | PBS/IgG4^{P} | REGN2477 | Semaglutide/IgG4^{P} | & REGN2477 |
| N | 8 | 7 | 8 | 8.682 |
| Mean | −7.143E−09 | 9.21 | 22.02 | 31.9 |
| Std. Deviation | 10.28 | 22.07 | 20.34 | 24.56 |
| Std. Error of Mean | 3.876 | 8.342 | 7.191 | 8.682 |

TABLE 13
Change in beta cell mass

| | | PBS/REGN1033 & | | Semaglutide/REGN1033 |
|---|---|---|---|---|
| | PBS/IgG4^{P} | REGN2477 | Semaglutide/IgG4^{P} | & REGN2477 |
| N | 8 | 7 | 8 | 9 |
| Mean | F | −45.74 | −16.12 | −38.83 |
| Std. Deviation | 49.35 | 32.04 | 50.64 | 29.02 |
| Std. Error of Maan | 18.65 | 12.11 | 17.9 | 10.26 |

TABLE 14
Change in alpha cell mass

| | | PBS/REGN1033 & | | Semaglutide/REGN1033 |
|---|---|---|---|---|
| | PBS/IgG4^{P} | REGN2477 | Semaglutide/IgG4^{P} | & REGN2477 |
| N | 8 | 7 | 8 | 9 |
| Mean | −1.286E−09 | −60.56 | −28.15 | −46.58 |
| Std. Deviation | 27.69 | 21.72 | 35.44 | 34.32 |
| Std. Error of Mean | 10.47 | 6.209 | 12.53 | 12.14 |

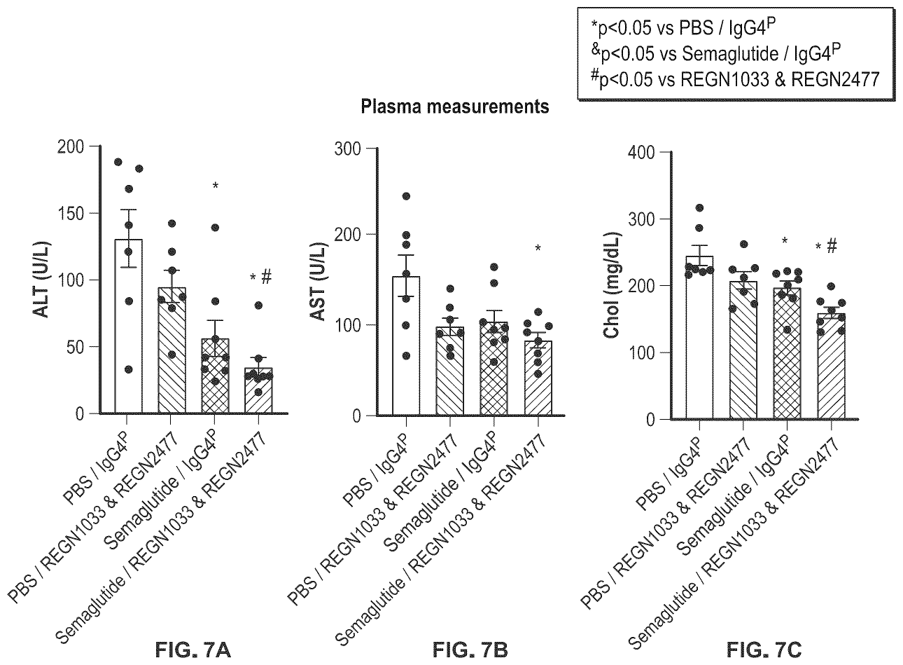

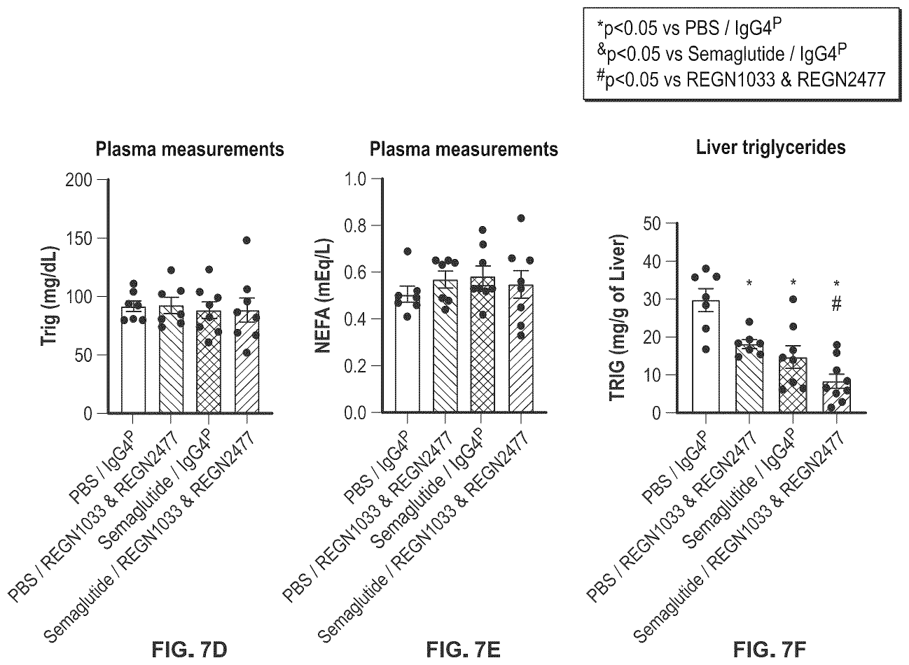

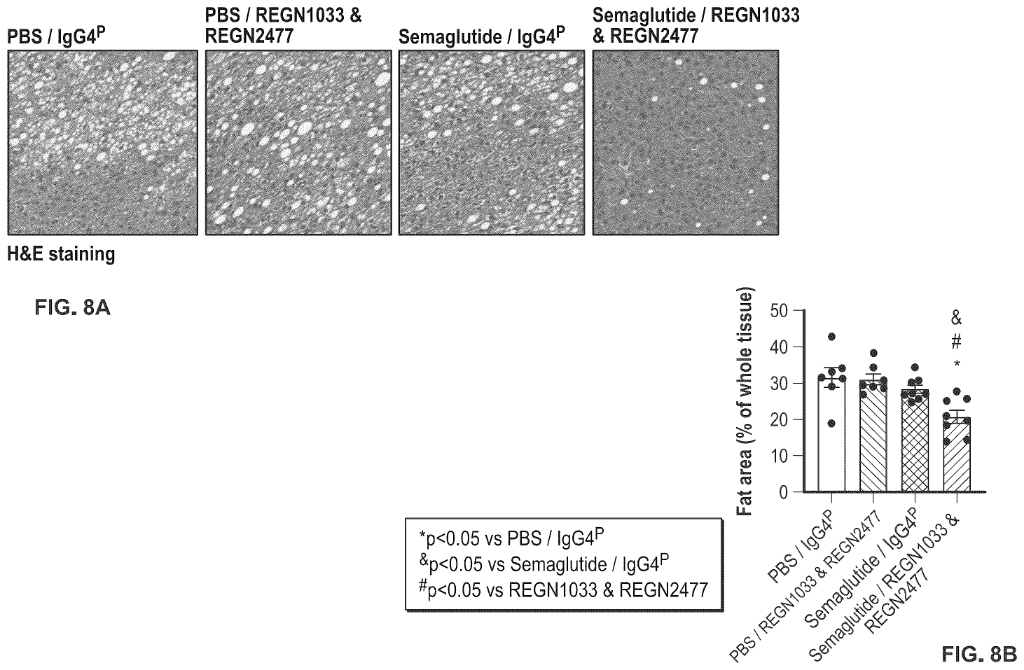

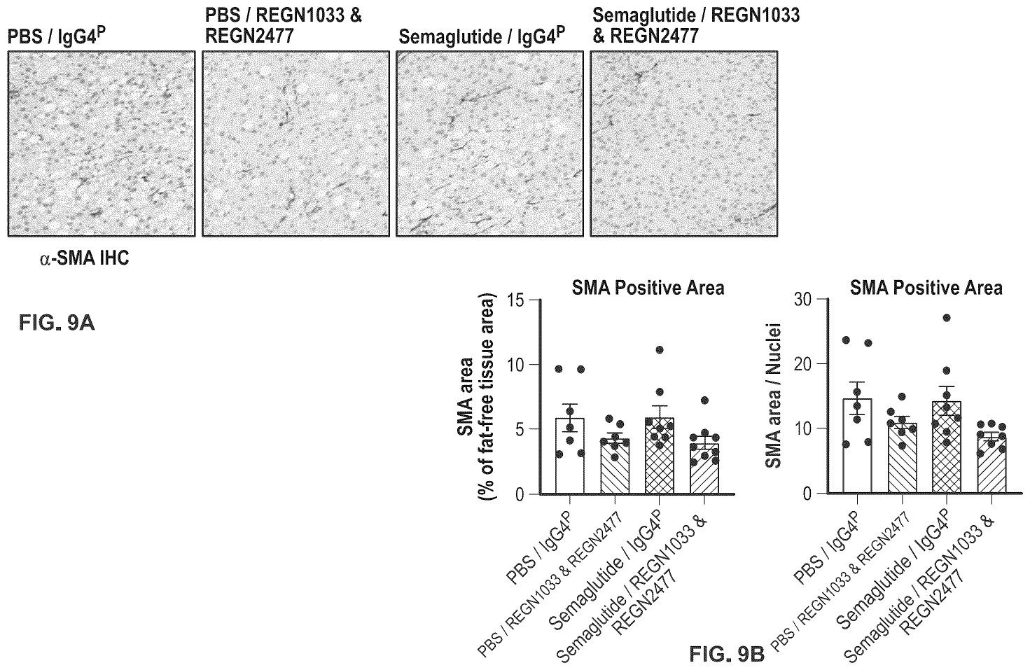

Since loss of adipose tissue is associated with improved liver phenotype, this was also evaluated. Plasma measurements of ALT and AST, markers of liver damage, showed a clear reduction with all treatments, however this was highest when REGN1033 & REGN2477 was combined with Semaglutide treatment (group 4) (FIGS. 7A and 7B). In addition, circulating cholesterol was reduced in group 4 (FIG. 7C). Correlating with the improvement of ALT and AST, all treatment groups had reduced liver triglyceride content (FIG. 7F). The combination of REGN1033 & REGN2477 with Semaglutide again seemed to have an additive effect in reducing liver triglycerides and ALT. Histological analysis of liver tissue confirmed these results on liver triglycerides. In addition, smooth-muscle actin staining, a marker for early fibrosis, was reduced in both groups treated with REGN1033 & REGN2477 (FIG. 9B), indicating that blocking Activin A and GDF8 could block liver fibrosis. There was no increase in circulating free fatty acids or triglycerides with any treatment, indicating that fat was burned and not just added into circulation.

TABLE 15
Plasma measurements - Liver panel

| | | | | PBS/REGN1033 & | | | Semaglutide/ | | | Semaglutide/REGN1033 & | | |
|---|---|---|---|---|---|---|---|---|---|---|---|---|
| | PBS/IgG4^{P} | | | REGN2477 | | | IgG4^{P} | | | REGN2477 | | |
| ALT | 131.1429 | 21.45586 | 7 | 95 | 12.00198 | 7 | [symbol] | 13.48074 | 8 | 34.75 | 7.02737 | 8 |
| AST | [symbol] | 23.03295 | 7 | 98.14255 | [symbol] | 7 | 103.625 | 12.2823 | 8 | 83 | [symbol] | 8 |
| Trig | [symbol] | 4.673707 | 7 | [symbol] | [symbol] | 7 | [symbol] | 7.210483 | 8 | [symbol] | [symbol] | 8 |
| Chol | 241.4286 | [symbol] | 7 | 204.7143 | [symbol] | 7 | 193.75 | [symbol] | 8 | 157.125 | [symbol] | 8 |
| NEFA | [symbol] | 0.033442 | 7 | [symbol] | [symbol] | 7 | 0.58375 | 0.042213 | 8 | 0.5475 | [symbol] | 8 |

[symbol]

indicates data missing or illegible when filed

TABLE 16
Liver triglycerides

| | | PBS/REGN1033 & | | Semaglutide/REGN1033 |
|---|---|---|---|---|
| | PBS/IgG4^{P} | REGN2477 | Semaglutide/IgG4^{P} | & REGN2477 |
| N | 8 | 2 | 8 | 9 |
| Mean | 260.8 | 171.4 | 153.6 | 119.6 |
| Std. Deviation | 58.29 | 21.75 | 56.12 | 50.77 |
| Std. Error of Mean | 22.03 | 8.219 | 19.84 | 16.92 |

TABLE 17
Liver fat area

| | | PBS/REGN1033 & | | Semaglutide/REGN1033 |
|---|---|---|---|---|
| | PBS/IgG4^{P} | REGN2477 | Semaglutide/IgG4^{P} | & REGN2477 |
| N | 8 | 7 | 8 | 9 |
| Mean | 31.52 | 30.99 | 28.34 | 20.68 |
| Std. Deviation | 7.101 | 3.946 | 3.16 | 5.187 |
| Std. Error of Mean | 2.684 | 1.492 | 1.117 | 1.834 |

TABLE 18
SMA positive area (% of fat-free area)

| | | PBS/REGN1033 & | | Semaglutide/REGN1033 |
|---|---|---|---|---|
| | PBS/IgG4^{P} | REGN2477 | Semaglutide/IgG4^{P} | & REGN2477 |
| N | 8 | 7 | 8 | 9 |
| Mean | 5.888 | 4.318 | 6.942 | 3.962 |
| Std. Deviation | 2.809 | 1.014 | 2.438 | 1.473 |
| Std. Error of Mean | 1.062 | 0.3834 | 0.8621 | 0.4909 |

TABLE 19
SMA positive area (normalized to nuclei amount)

| | | PBS/REGN1033 & | | Semaglutide/REGN1033 |
|---|---|---|---|---|
| | PBS/IgG4^{P} | REGN2477 | Semaglutide/IgG4^{P} | & REGN2477 |
| N | 8 | 7 | 8 | 9 |
| Mean | 14.72 | 10.84 | 14.32 | 8.79 |
| Std. Deviation | 6.82 | 2.425 | 6.237 | 1.767 |
| Std. Error of Mean | 2.502 | 0.9166 | 2.205 | 0.6249 |

Overall, these data indicate that the combination of REGN1033 & REGN2477 with Semaglutide had additive effects on fat loss and improvement of liver disease (lowering signs of liver damage (ALT/AST), lower liver triglycerides, lower steatosis and fibrosis).

### Example 2: Efficacy Study to Examine the Effect of Adding Anti-Myostatin Treatment or Adding Anti-Myostatin and Anti-Activin A Treatment Onto Treatment With GLP-1 Agonist

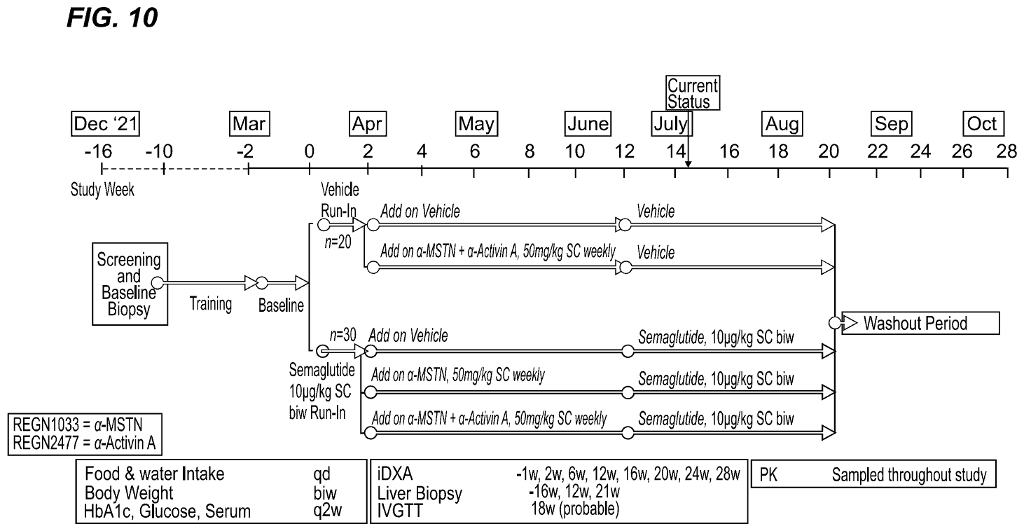

An obese NHP study of the body weight, liver and metabolic effects of adding on myostatin/activin A blockade onto GLP-1R agonism was carried out. FIG. 10 sets forth the study and timeline for obese adult male cyno non-human primate (NHP) with NASH, stratified based on body weight and % body fat, n=10 per arm. NHP have been shown to spontaneously develop obesity, metabolic syndrome, and type 2 diabetes, and their metabolic features mirror humans (Canter, et al., 2018, J Leukoc Biol 104(3):487-497).

The treatment groups were:
vehicle;
Semaglutide;
Anti-Myostatin+anti-Activin A;
Semaglutide+anti-Myostatin; and
Semaglutide+anti-Myostatin+anti-Activin A.
   REGN1033 is an anti-Myostatin antibody, and REGN2477 is an anti-Activin A antibody. The GLP-1 agonist employed was Semaglutide. Semaglutide was administered at 10 μg/kg. Anti-Myostatin antibody was administered at 50 mg/kg. Anti-Activin A antibody was administered at 50 mg/kg.

### Results

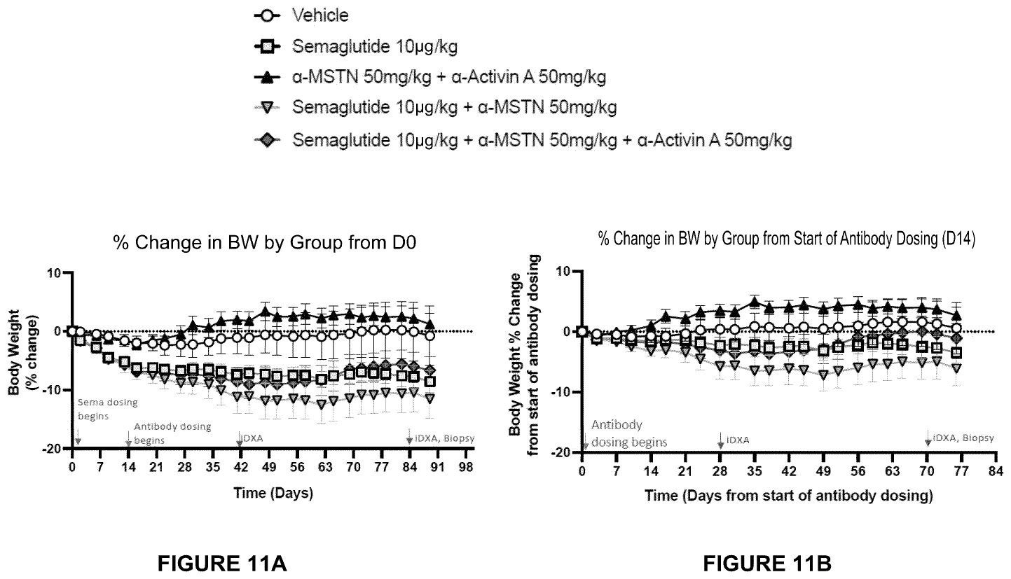

Anti-Myostatin treatment added onto Semaglutide led to increased weight loss in comparison with Semaglutide monotherapy (FIGS. 11A and 11B).

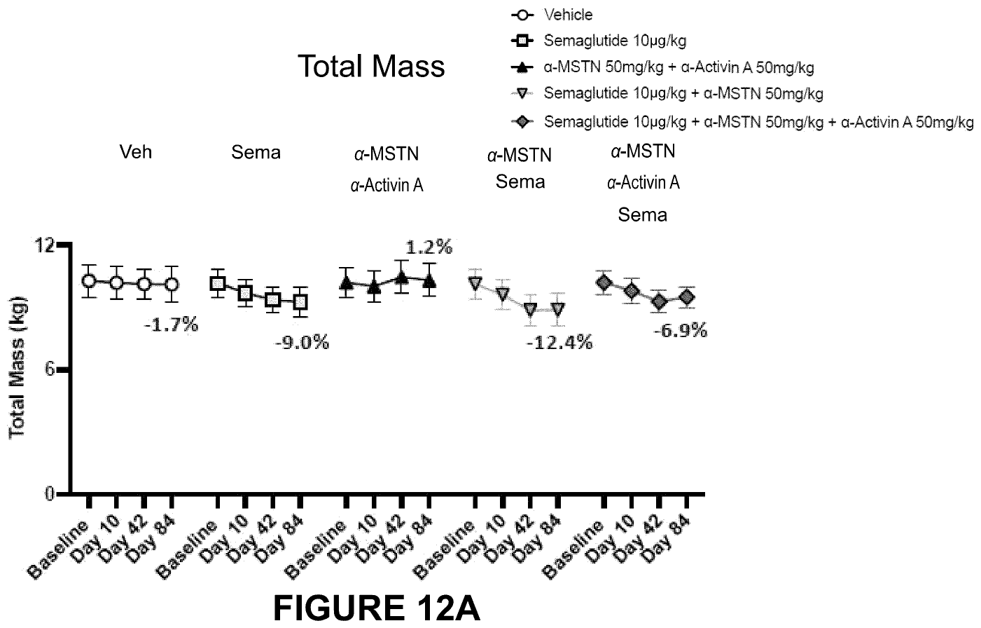

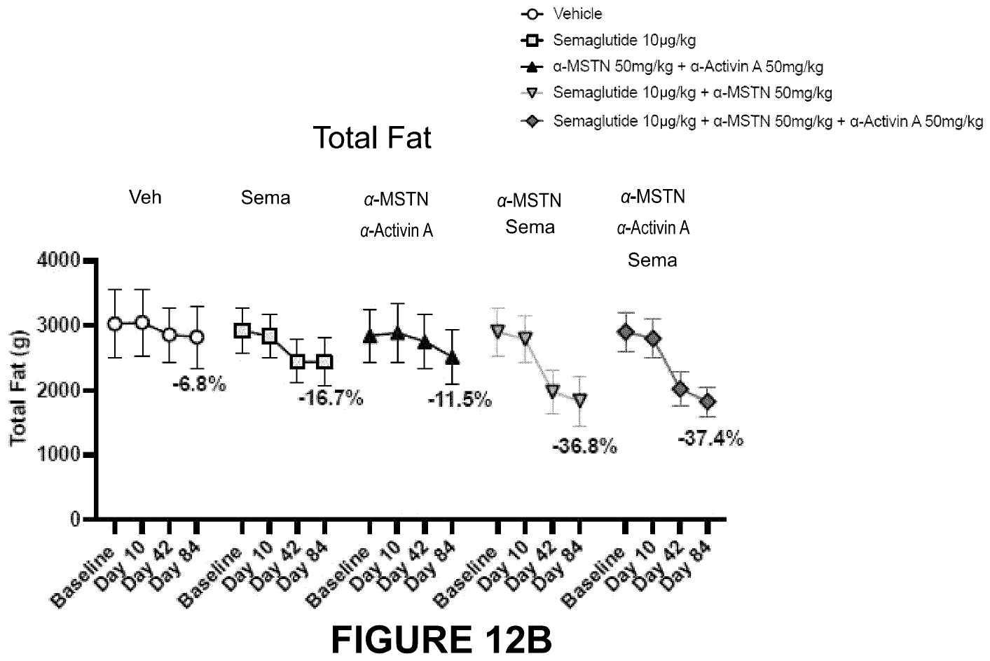

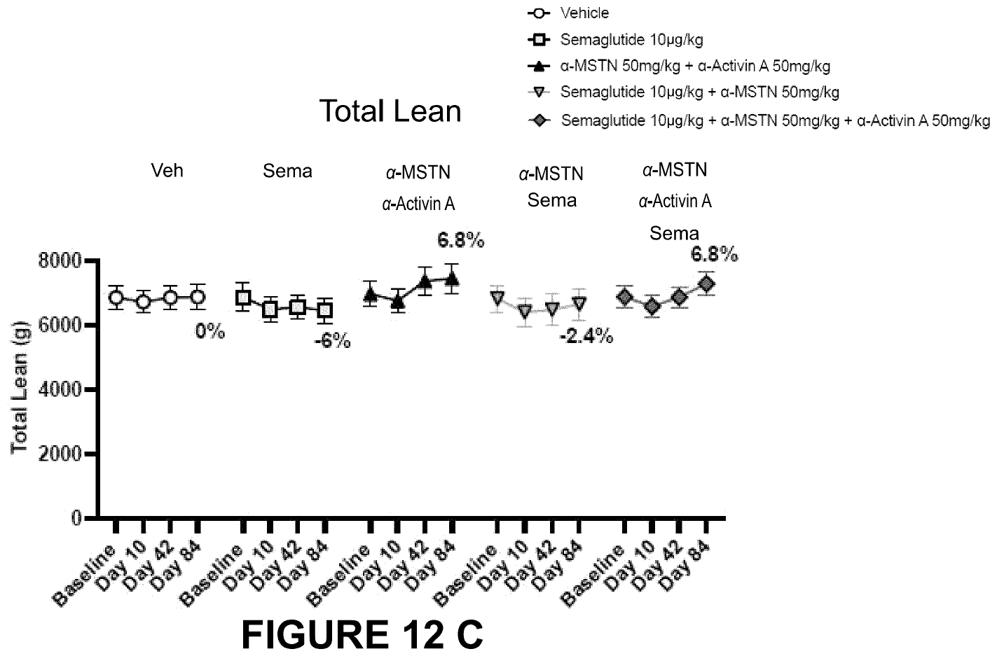

Anti-Myostatin treatment added onto Semaglutide led to increased fat loss in comparison with Semaglutide monotherapy, and anti-Activin A added to anti-Myostatin+Semaglutide treatment also increased lean mass (FIGS. 12A-12C). At week 12, the addition of anti-myostatin (REGN1033) to Semaglutide had increased the amount of fat loss over Semaglutide alone (FIG. 12B). The further addition of anti-activin A (REGN2477) also restored the lean mass loss seen with Semaglutide and further increased lean mass over baseline at week 12 (FIG. 12C).

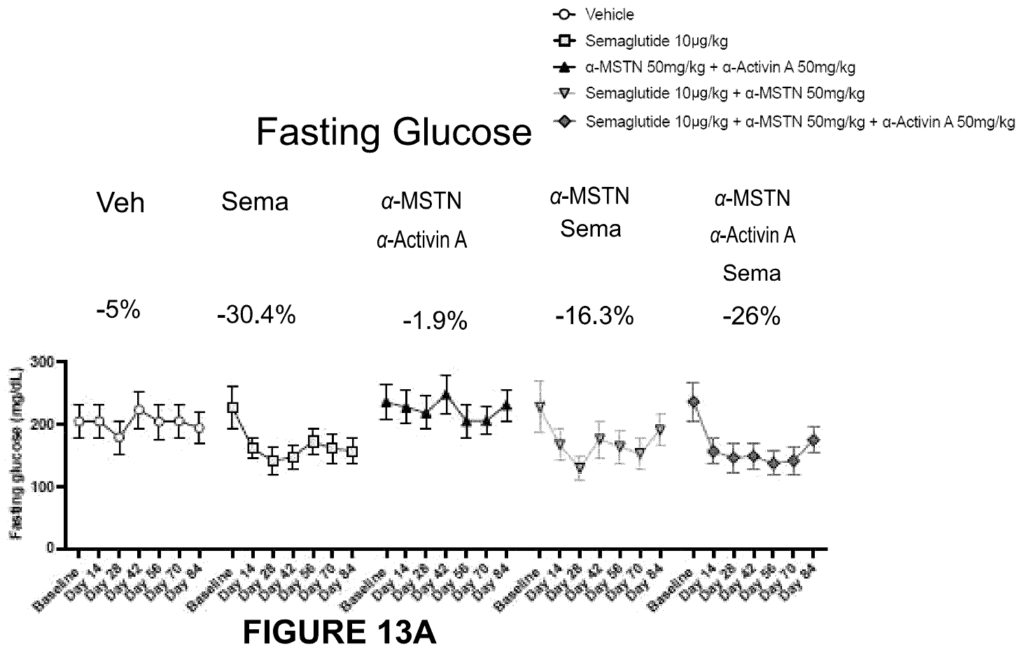

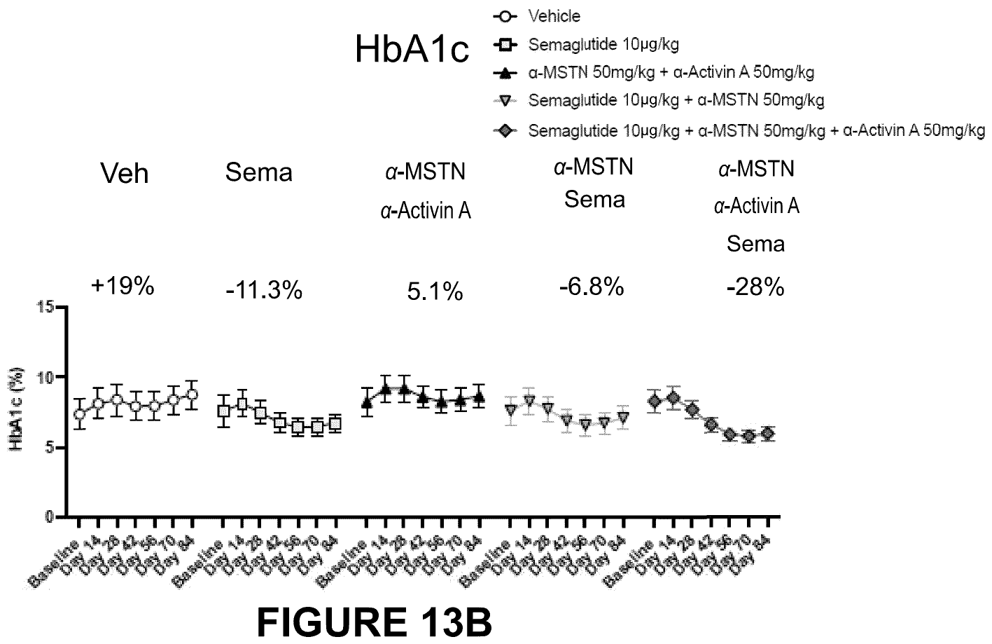

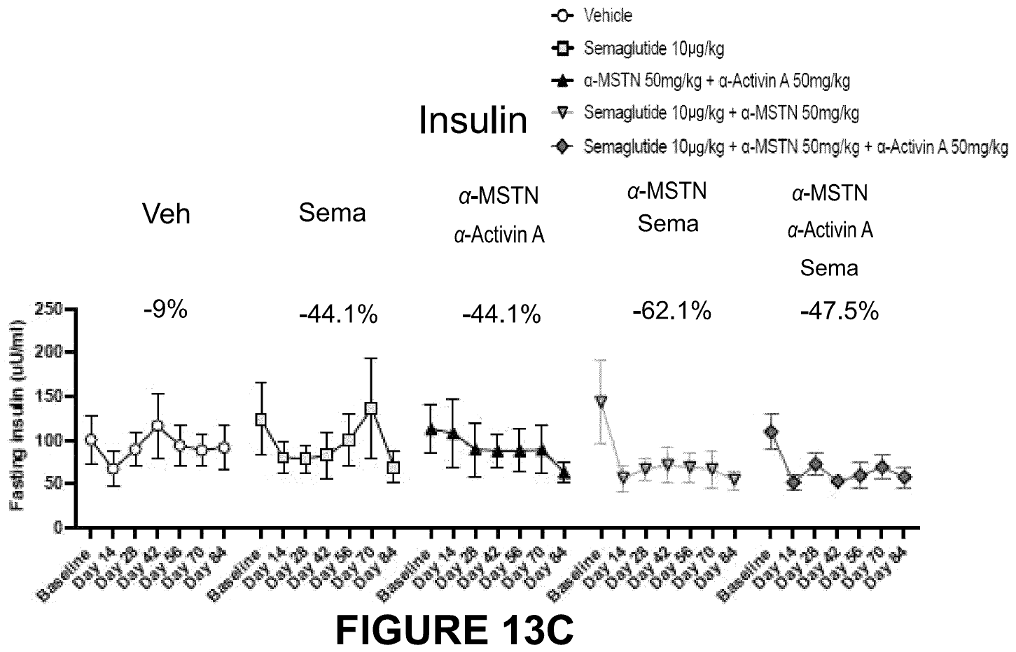

The triple combination of Semaglutide, anti-Myostatin antibody, and anti-Activin A antibody exhibited the largest reduction in HbA1c % after 12 weeks of treatment (FIGS. 13A-13C).

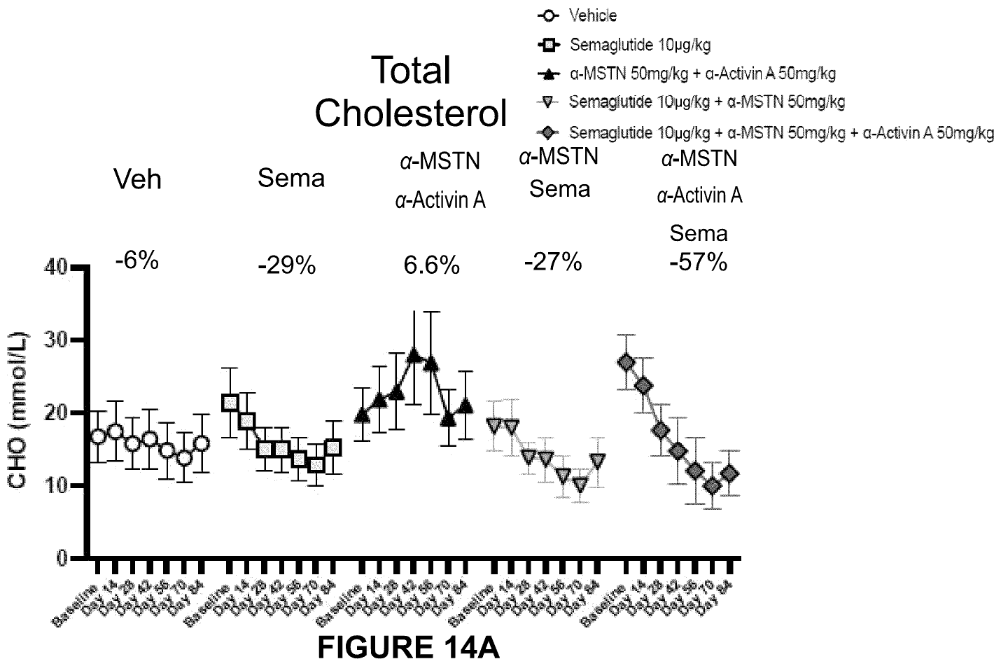

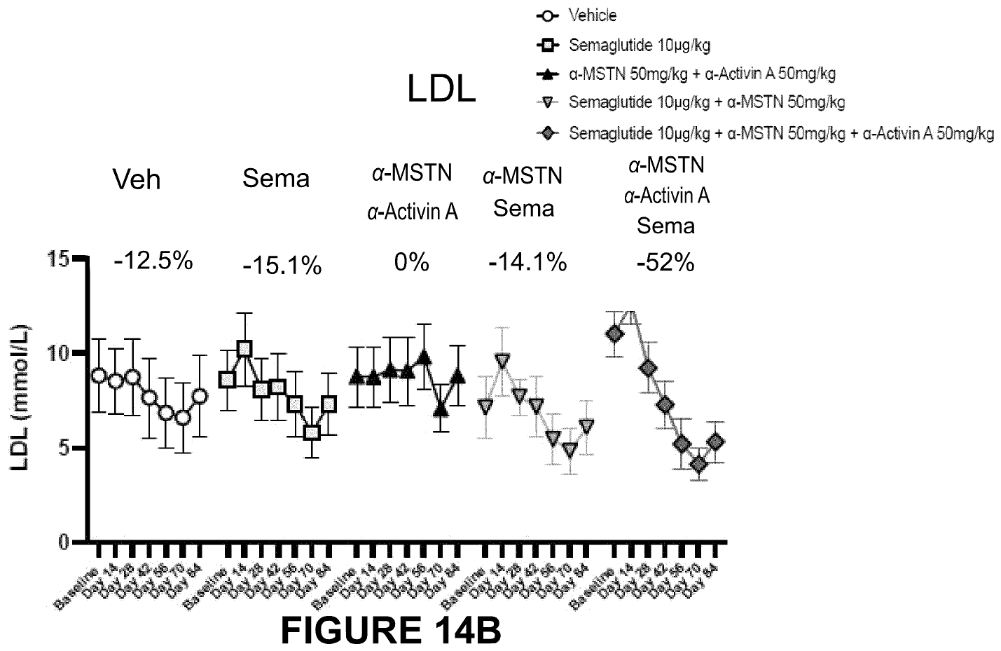

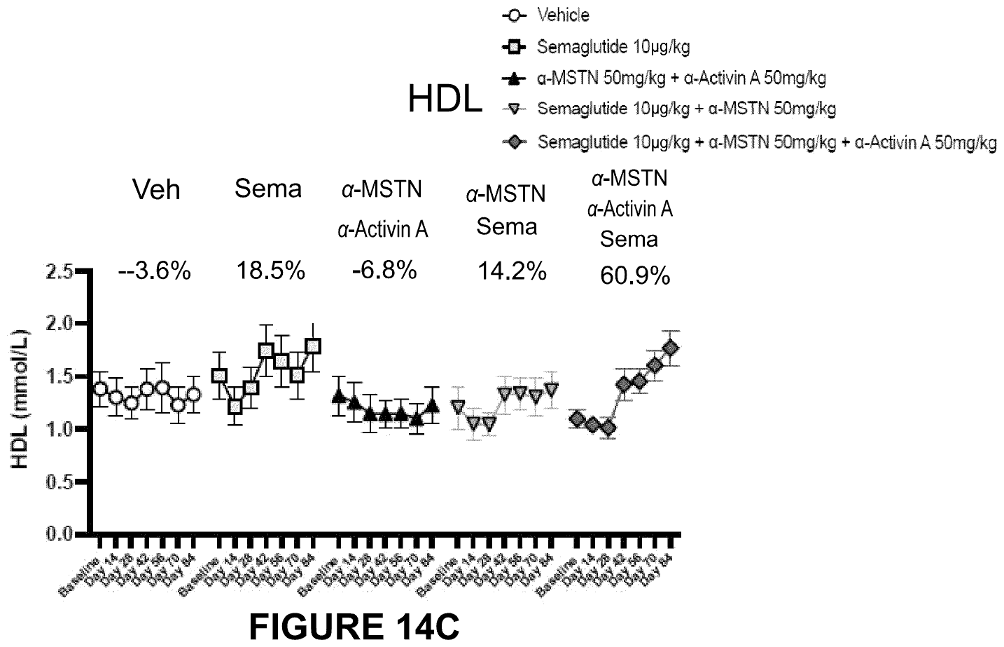

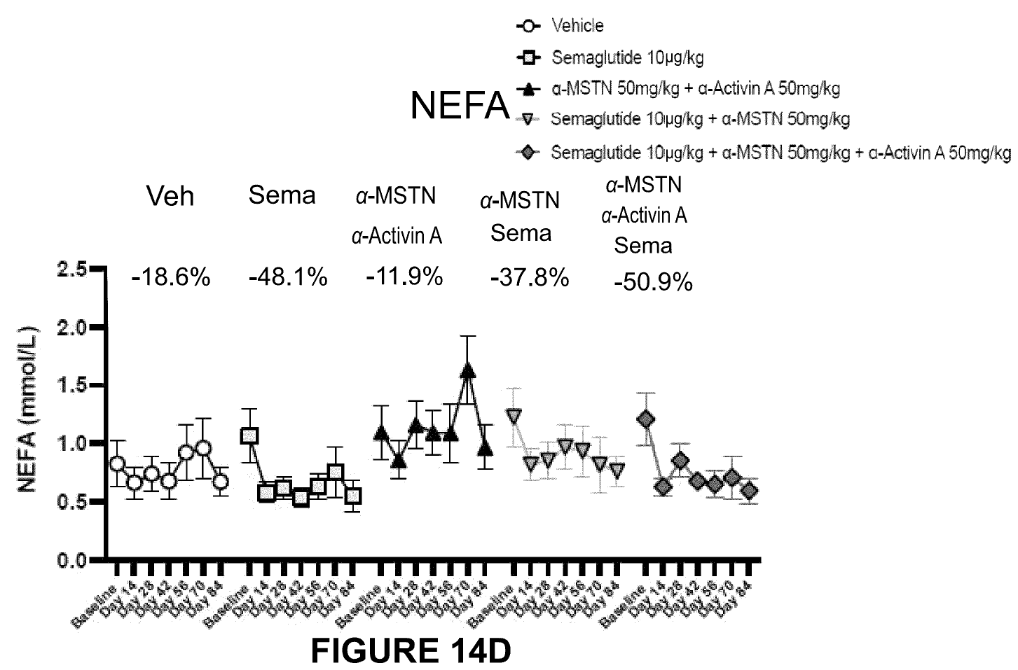

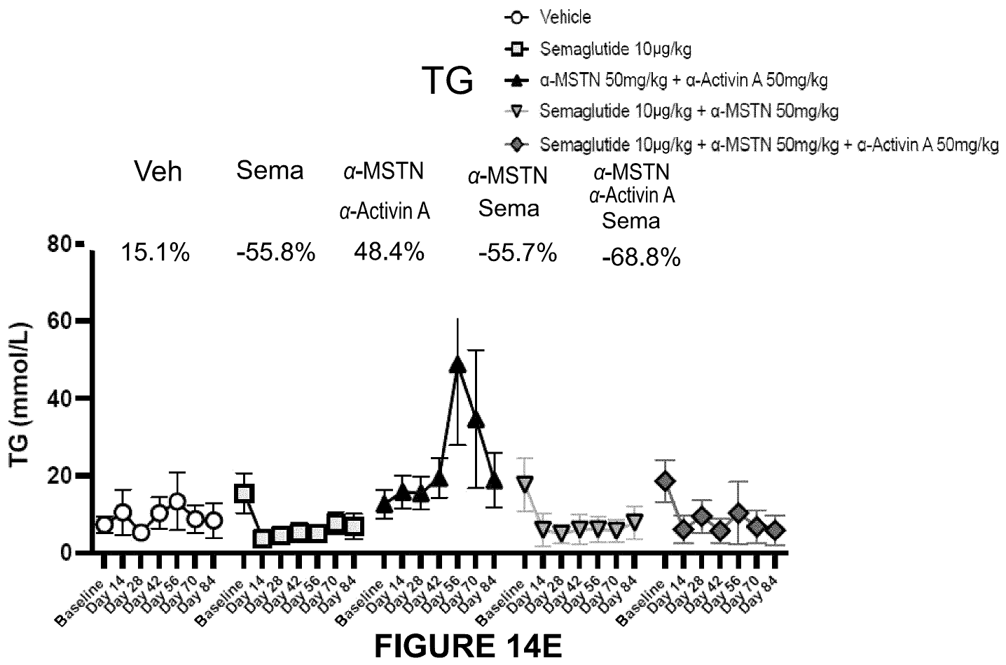

The triple combination of Semaglutide, anti-Myostatin antibody, and anti-Activin A antibody exhibited the largest reduction in LDL, the largest increase in HDL after 12 weeks of treatment (FIGS. 14A-14E).

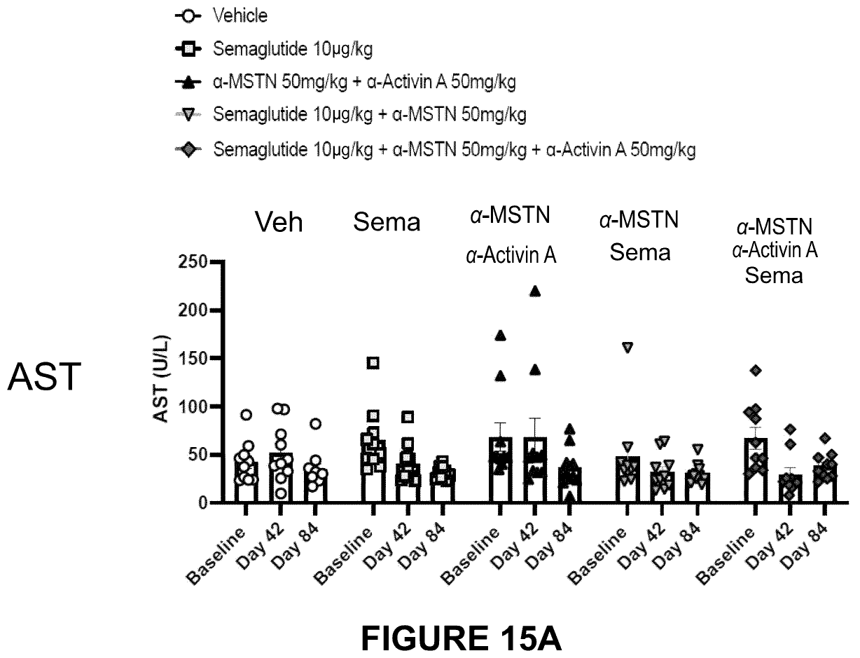

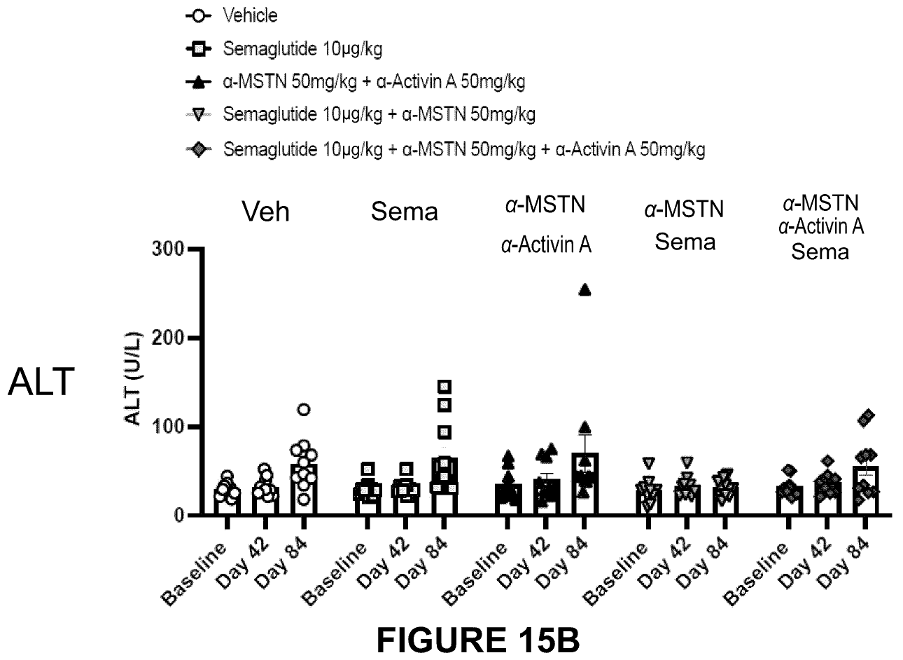

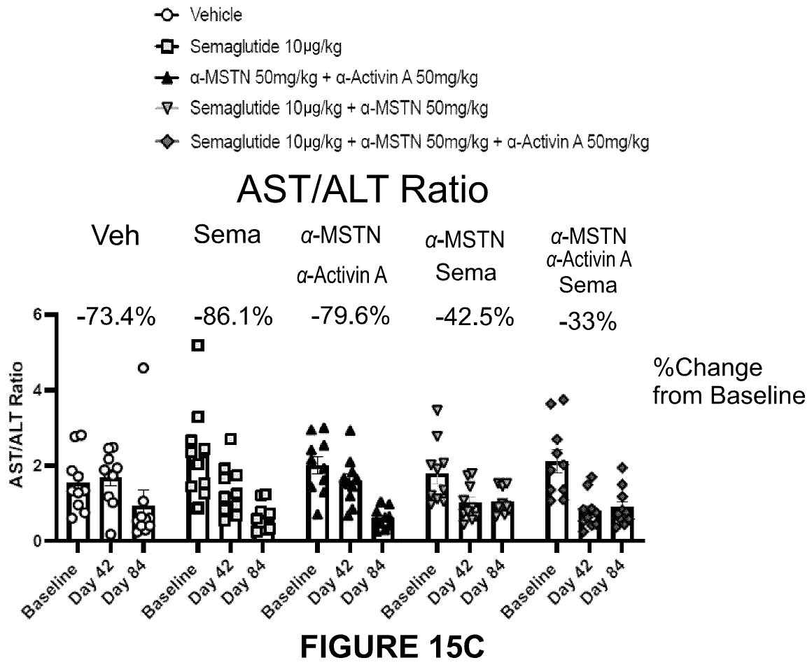

All groups had improvements in AST/ALT ratio at week 12 (FIGS. 15A and 15B).

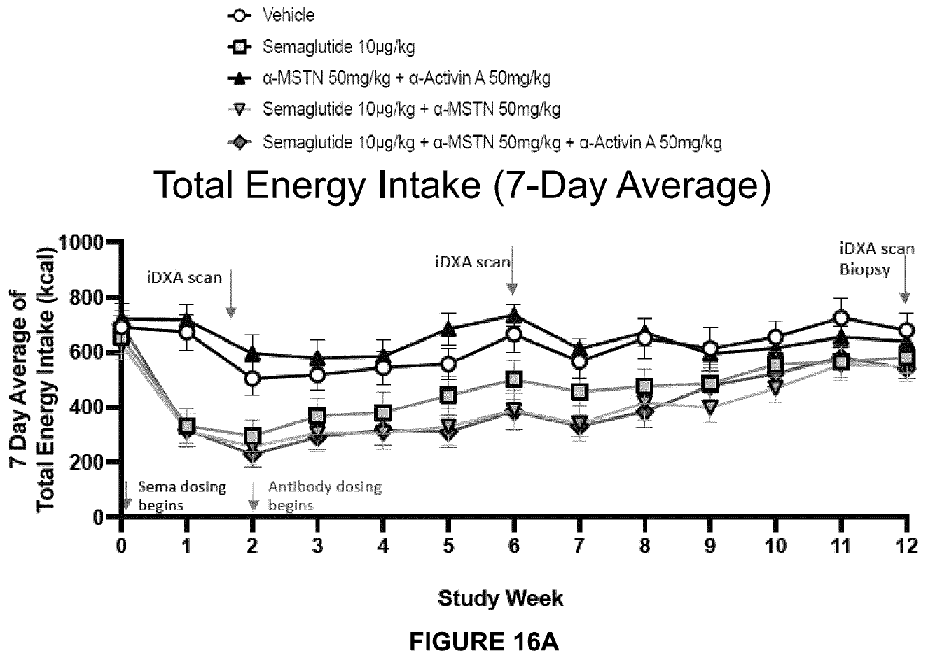

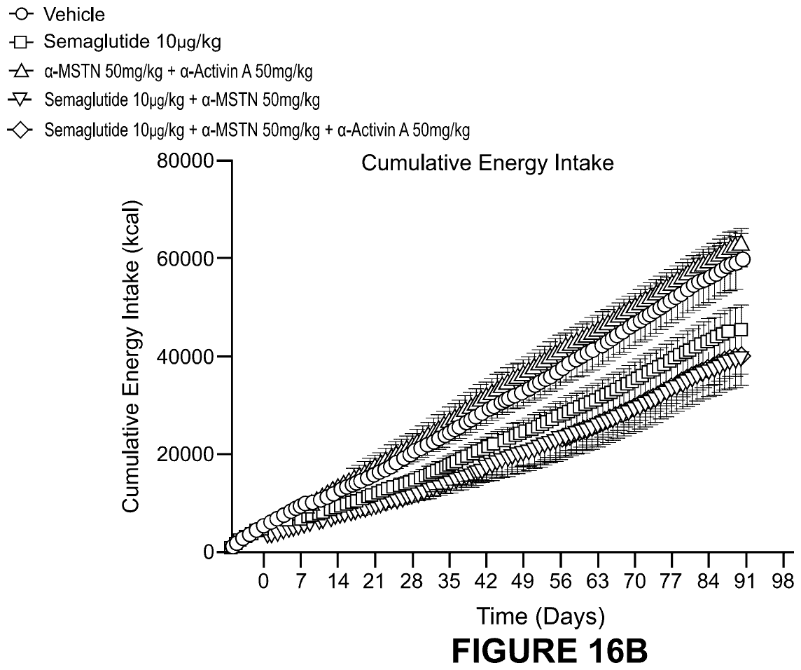

All groups given Semaglutide had lower food intake following initial dose, but the Semaglutide control group had a faster return to baseline (FIGS. 16A and 16B). Anti-Myostatin and anti-Activin A may have slightly increased food intake at the start of dosing, though this change did not widen with time, as seen by week 12. However, the addition of anti-Myostatin to Semaglutide may help delay the return to baseline of Semaglutide's appetite suppressing effects, as observed at week 12.

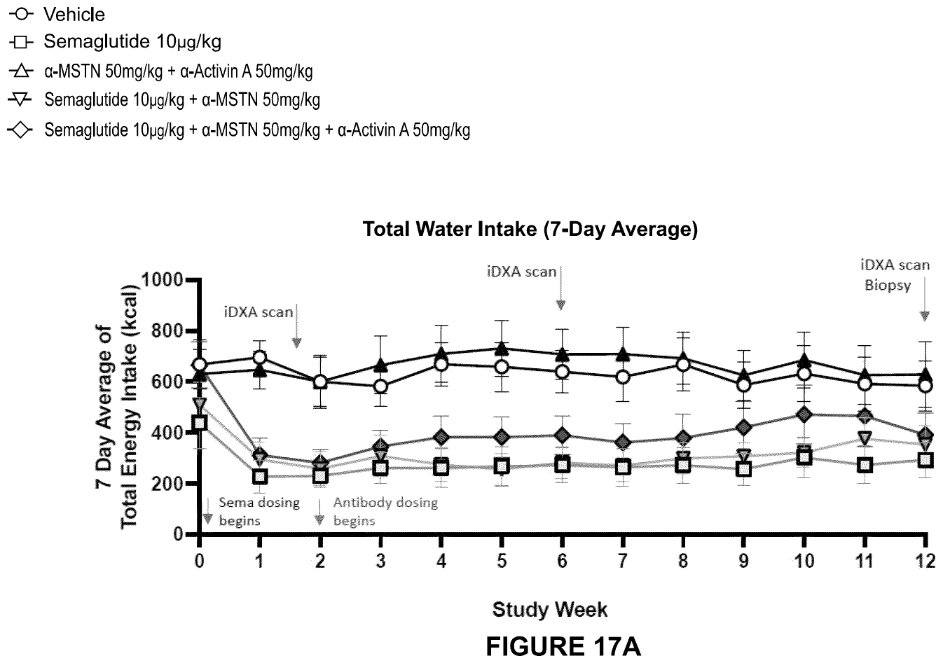

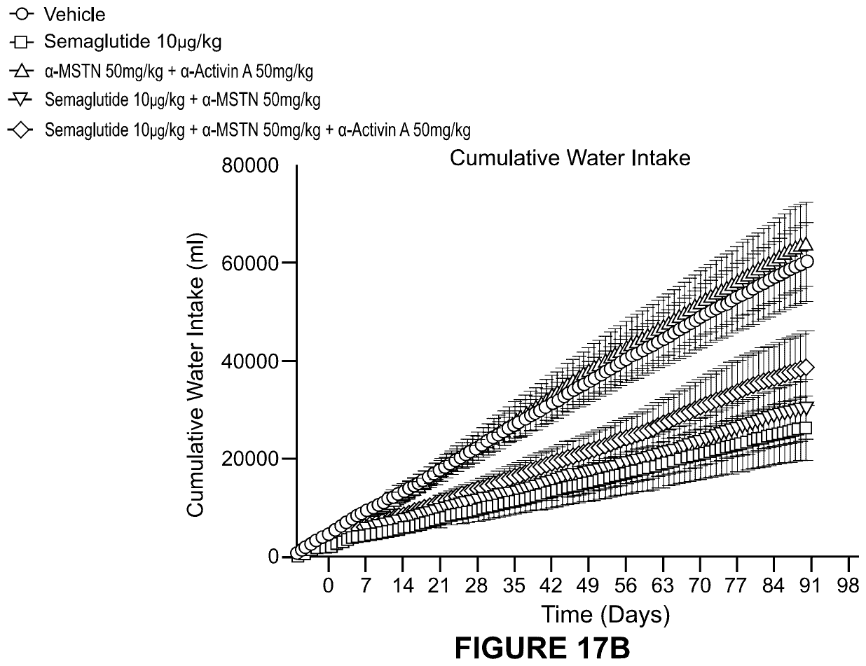

All groups given Semaglutide had lower water intake following the initial dose, but the triple combination group was not quite as suppressed as the anti-Myostatin+Semaglutide combination (FIG. 17A). All Semaglutide-treated groups had lower water intake from the start of dosing (through week 12). However, the addition of anti-activin A on top of anti-Myostatin and Semaglutide reduced this thirst suppression.

Thus, it was shown that:
Semaglutide and Anti-Myostatin have a synergistic effect on fat loss;
The addition of Anti-Activin A further increases lean mass and also further improves fasting glucose, HbA1c, LDL, and HDL;
Anti-Myostatin and Anti-Activin A alone shows some fat loss and similar lean mass gain to the triple combination, but fails to improve circulating lipids and glucose; and
Food intake was reduced in all Semaglutide-treated groups.

### Example 3. Blockade of Myostatin and Activin A in Non-Human Primates and Humans

Postmenopausal women received 10 mg/kg (anti-myostatin+anti-activin A) IV, Q2W. Thigh muscle volume was measured via MRI, and android fat mass was measured via iDXA, both over 30 weeks. Obese non-human primates received 50 mg/kg anti-myostatin+50 mg/kg anti-activin A (QW). Total lean mass was measured via iDXA, and total fat mass was measured via iDXA, both over 28 weeks.

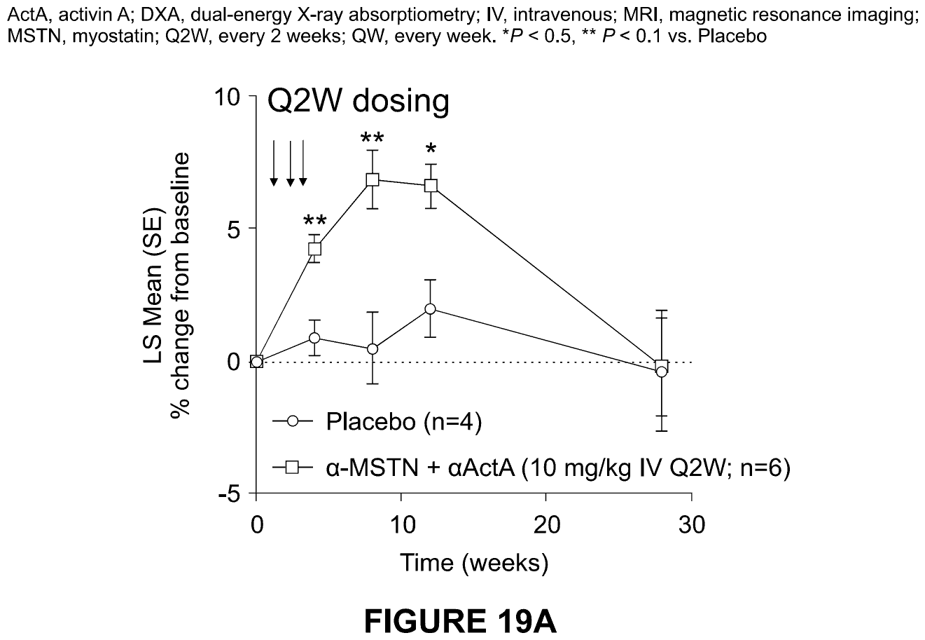

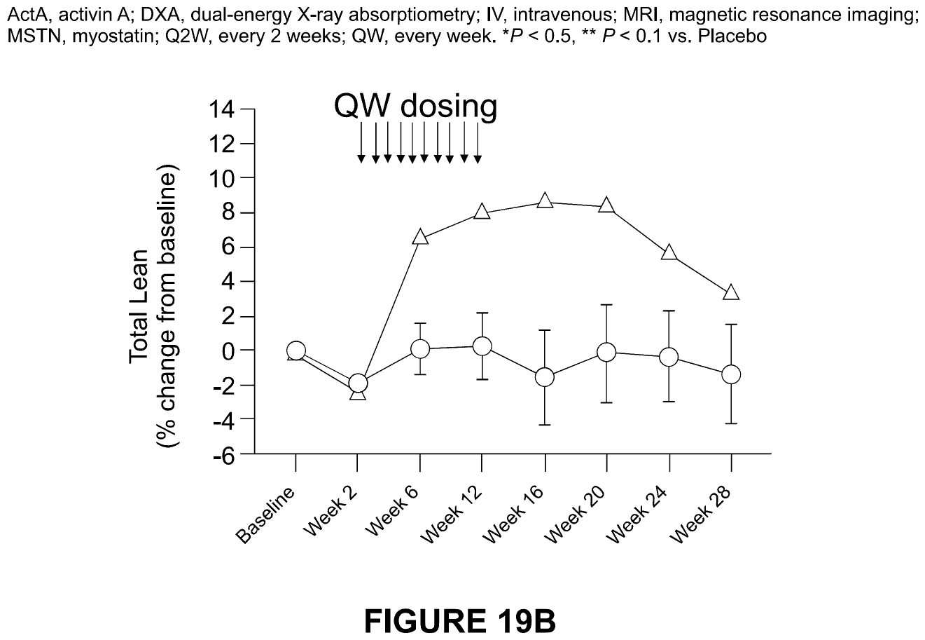

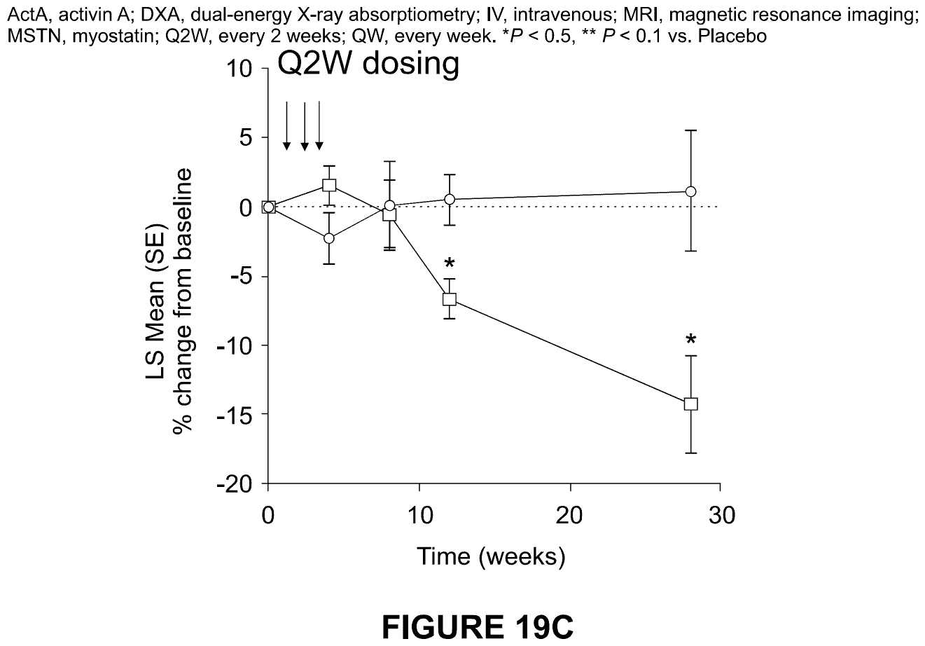

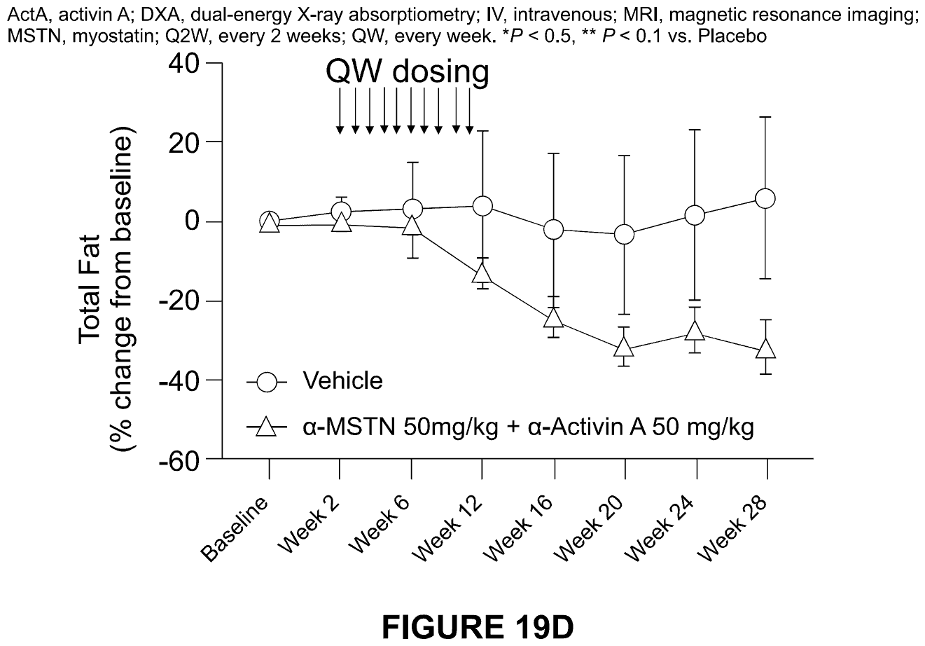

Myostatin and activin A inhibition resulted in a steady increase in thigh muscle volume (MRI) over the first ten weeks treatment in postmenopausal women (FIG. 19A), as well as in a (relatively) steady increase in total lean mass (iDXA) in obese non-human primates (FIG. 19B). At the same time, myostatin and activin A inhibition resulted in a steady decrease in android fat mass (iDXA) over 30 weeks treatment in postmenopausal women (FIG. 19C), as well as in a steady decrease in total fat mass (iDXA) over 28 weeks treatment in obese non-human primates (FIG. 19D).

Myostatin and activin A inhibition led to significant fat loss in both humans and non-human primates, but not until after significant lean mass gain. However, the effects on fat persisted after lean mass returned to baseline.

### Example 4. Addition of Blockade of Myostatin and Activin A to Semaglutide (GLP-1R Agonist) Increases Energy Expenditure in Obese Non-Human Primates

In this diet-induced obese non-human primate study, 64 male, obese monkeys naïve to human immunoglobulin were given initial selection screening with a physical examination, metabolic and safety clinical chemistry profiling, hematology, iDEXA (using a GE Lunar iDXA scanner) for body composition and a liver biopsy for nonalcoholic fatty liver disease plus fibrosis (NAS+) scoring. Animals were requested to have body fat>25%, fasting glucose≤350 mg/dl and ≥100 mg/dL, a liver NAS+score greater than 4 with a steatosis score of greater than 2. From this cohort, 55 monkeys were selected to move to diet transition from the high-fat diet on which they were maintained to a high-fat, high-fructose diet that would exacerbate any liver disease. Animals were maintained on this diet for six weeks, after which they underwent baseline screening for the study with an additional physical examination, metabolic and safety clinical chemistry profiling, hematology, iDEXA (using a GE Lunar iDXA scanner) for body composition. Food and water intake were also monitored daily. Six days of daily food and water intake were averaged for the baseline readings of those parameters.

A final cohort of fifty monkeys were selected and balanced across five groups with an n=10 each based on the following parameters: Body weight, total body fat and NAS+ score. Secondary parameters which were considered were fasting glucose, total lean mass and fasting triglycerides. The baseline characteristics of the five groups are listed in Table 20.

TABLE 20
Baseline characteristics

| | | | | Semaglutide + | Semaglutide + |
|---|---|---|---|---|---|
| | Vehicle | Semaglutide | α-MSTN/α-ActA | α-MSTN | α-MSTN/α-ActA |
| Age (yr) | 17.3 ± 1.3 | 18.3 ± 1.1 | 15.4 ± 0.8 | 17.5 ± 1.0 | 17.4 ± 1.3 |
| Body Weight (kg) | 9.9 ± 0.5 | 10.2 ± 0.7 | 9.6 ± 0.7 | 10.3 ± 0.8 | 10.2 ± 0.6 |
| Total Fat Mass (kg) | 2.9 ± 0.4 | 3.0 ± 0.4 | 2.4 ± 0.4 | 2.9 ± 0.4 | 2.9 ± 0.4 |
| Total Lean Mass (kg) | 6.7 ± 0.3 | 6.8 ± 0.4 | 7.0 ± 0.4 | 6.9 ± 0.4 | 6.9 ± 0.3 |
| Fasting Glucose | 156 ± 22 | 201 ± 36 | 191 ± 33 | 174 ± 26 | 188 ± 30 |
| (mg/dL) | | | | | |
| Fasting TG (mmol/L) | 5.8 ± 2.2 | 6.3 ± 2.4 | 9.2 ± 5.1 | 6.0 ± 2.9 | 8.5 ± 4.1 |
| NAS+ Score | 4.9 ± 0.4 | 4.9 ± 0.4 | 5.0 ± 0.4 | 5.2 ± 0.3 | 5.1 ± 0.4 |

Average ± SEM

### Compounds and Treatment Groups

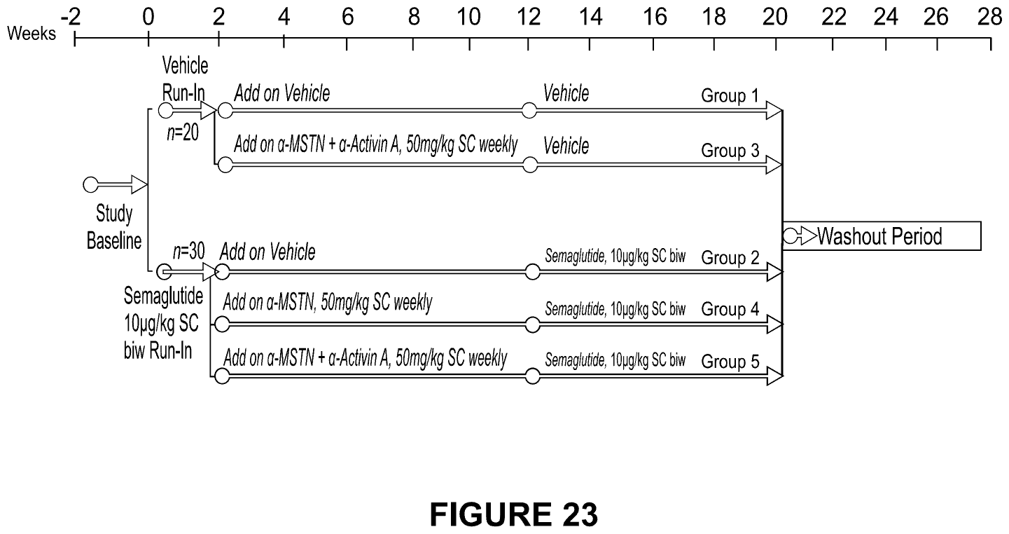

Grouping and dosing for the study are listed in Table 21, below, and a schema of the design is shown in FIG. 23. There were five groups in the study: Vehicle control, GLP-1, α-STN and α-ActA, GLP-1+α-MSTN and GLP-+α-MSTN+α-ActA. All compounds were dosed subcutaneously. GLP-1 was given twice per week for 20 weeks starting on Day 0 of the study at 10 ug/kg and no titration of GLP-1 was given due to the low dose regimen. Groups given α-MSTN and α-ActA were dosed weekly at 50 mg/kg starting Week 2 of the study until Week 12. All dosing was completed by Week 20 after which the cohort was followed for an additional eight weeks for a washout period. The number of animals listed in Table 21 represents the final number at the end of the study. There were four deaths which occurred during the study; all data from those monkeys were excluded from the averages unless otherwise noted.

TABLE 21
Study groups and dosing

| Group | Compound and Dose | Frequency | Final N |
|---|---|---|---|
| 1) Vehicle | Vehicle | Twice weekly for 20 weeks | 10 |
| 2) GLP-1 | Semaglutide 10 μg/kg | Twice weekly for 20 weeks | 9 |
| 3) α-MSTN + α-ActA | REGN1033 50 mg/kg | Once weekly for 10 weeks | 8 |
| | REGN2477 50 mg/kg | starting week 2 | |
| 4) GLP-1 + α-MSTN | Semaglutide 10 μg/kg | Twice weekly for 20 weeks | 9 |
| | REGN1033 50 mg/kg | Once weekly for 10 weeks | |
| | | starting week 2 | |
| 5) GLP-1 + α-MSTN + | Semaglutide 10 μg/kg | Twice weekly for | 10 |
| α-ActA | | 20 weeks | |
| | REGN1033 50 mg/kg + | Once weekly for 10 weeks | |
| | REGN2477 50 mg/kg | starting week 2 | |

### Liver Histology and Scoring

Liver biopsy was conducted twice during the study: once during the animal selection period and once at the end of week 12. Liver tissue samples (at ˜0.5 to 1.0 cm/sample) were obtained by biopsy from animals following an overnight fast under sedation/anesthesia (Ketamine, 5-10 mg/kg IM) under ultrasound guidance. For all samples (per time) after appropriately localizing the right lobe of liver by ultrasound, the biopsy needle is advanced slowly through the skin, muscle tissue, and the liver capsule to reach into the liver tissue and collected using a 16-18G biopsy needle under direct ultrasound guidance. The biopsy was fixed in 10% neutral buffered formalin for paraffin embedding. The paraffin embedded tissue samples were cut and stained with Hematoxylin and Eosin (H&E) and Sirius Red at the same time. The slides were evaluated for NASH (steatosis, ballooning, inflammation and fibrosis) by a KBI pathologist.

### Double-Labeled Water

Total Energy Expenditure (TEE) was be measured using the doubly labeled water method. Subjects were dosed with a 1.51 g/kg estimated total body water (TBW) of DLW. The dose was composed of 0.17 g/kg estimated TBW of 99% 2H2O (Sigma-Aldrich 151882) and 0.3 g/kg estimated TBW of 97% H218O (Sigma-Aldrich 329878) to achieve initial enrichments for their body mass. Doses were administered intravenously via a saphenous vein catheter or equivalent and syringes were weighed and flushed to ensure all compound was injected. Blood was sampled at the following timepoints: 1) Prior to dosing (baseline), 2) 4-6 hrs after dosing (equilibration) and 3) 1 W and 2 W after dosing.

Blood samples (1.5 ml) were collected and transferred to non-liquid sodium heparin anti-coagulated tubes, inverted 5 times and immediately placed on ice prior to centrifugation at 1300×g for 10 minutes at 4° C. Aliquots were stored at −80° C. and sent to Metabolic Solutions, Inc. for measurement of the labels and analysis.

In summary, obese non-human primates received either i) vehicle, ii) semaglutide, iii) anti-myostatin antibody+anti-activin A antibody, iv) semaglutide+anti-myostatin antibody, or v) semaglutide+anti-myostatin antibody+anti-activin A antibody. Energy expenditure was assessed by DLW conducted between weeks 18 and 20 of the study, at the end of the dosing period.

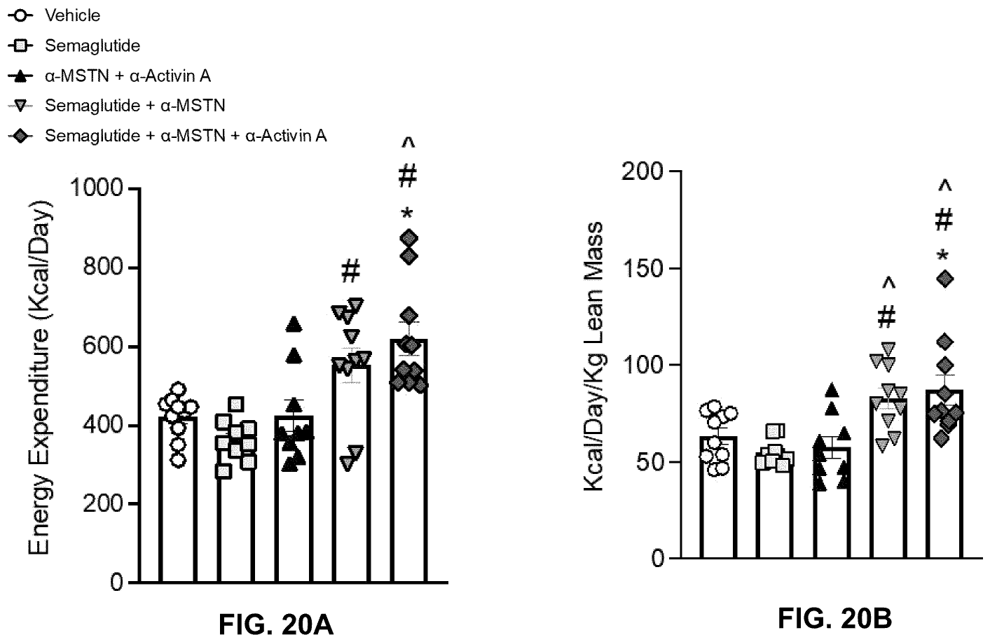

Raw energy expenditure measurements showed that the primate group receiving garetosmab (anti-activin A antibody)+semaglutide+anti-myostatin antibody expended more energy (FIG. 20A). Energy expenditure per kg lean mass measurements showed that the energy increase held true for semaglutide+trevogrumab (anti-myostatin antibody REGN1033), even when normalized per kg of lean mass (FIG. 20B).

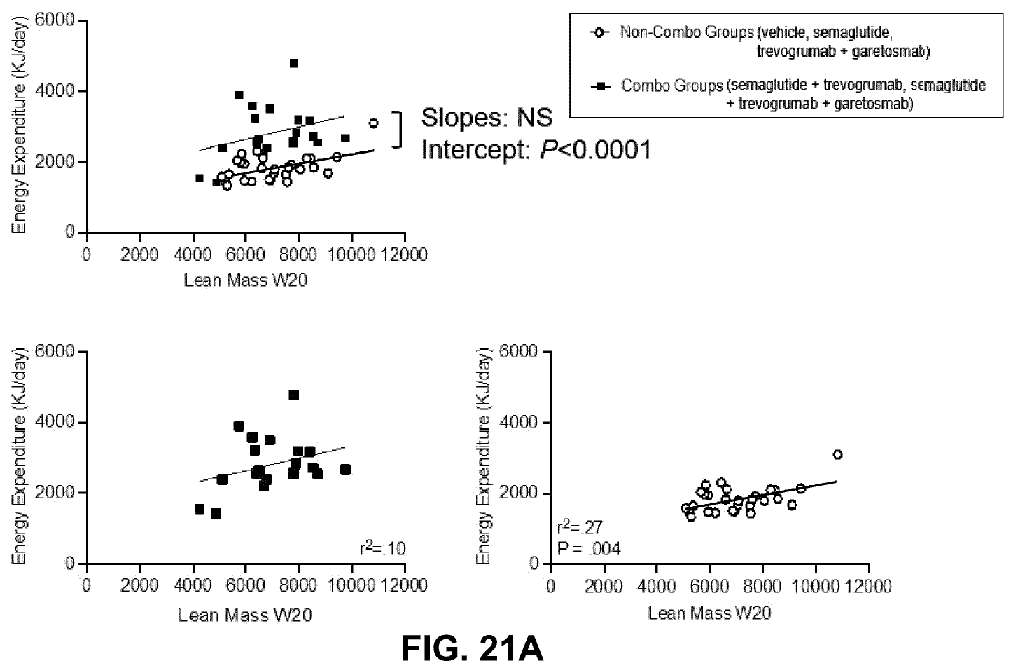

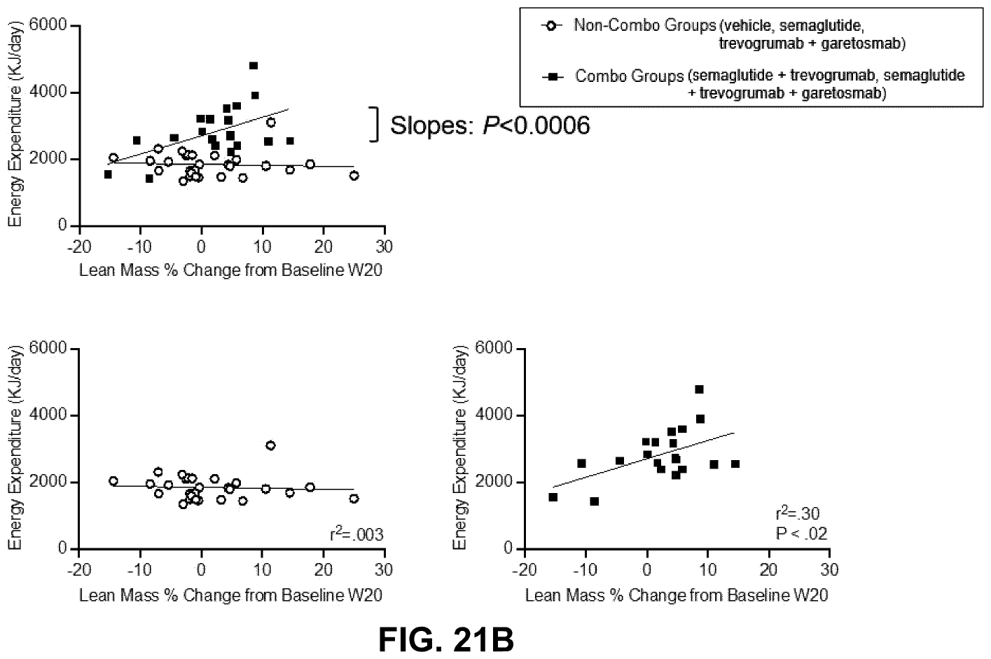

The data were graphed in order to determine whether energy expenditure correlates with lean mass or change in lean mass. The combination treatment groups (semaglutide+trevogrumab, semaglutide+trevogrumab+garetosmab (REGN2477)) expended more energy than the non-combination treatment groups (vehicle, semaglutide, trevogrumab+garetosmab) (FIG. 21A). Furthermore, the combination treatment groups (semaglutide+trevogrumab, semaglutide+trevogrumab+garetosmab) who gained more lean mass had greater energy expenditure (FIG. 21B). Thus, the combination treatment primates had higher energy expenditure per gram of lean mass and increased their energy expenditure in proportion to their lean mass gain. However, the energy expenditure “boost” only happened when both semaglutide and trevogrumab were on board.

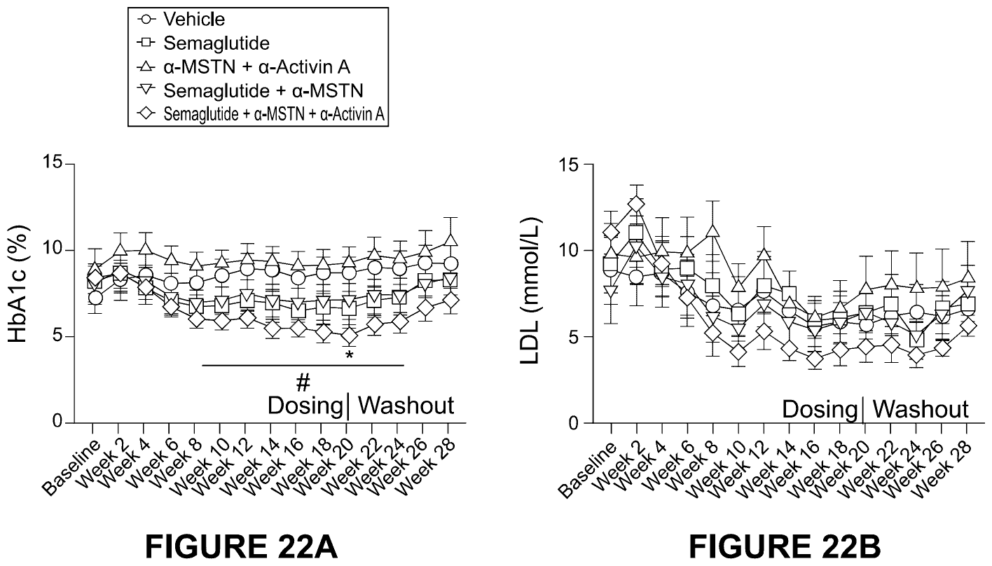

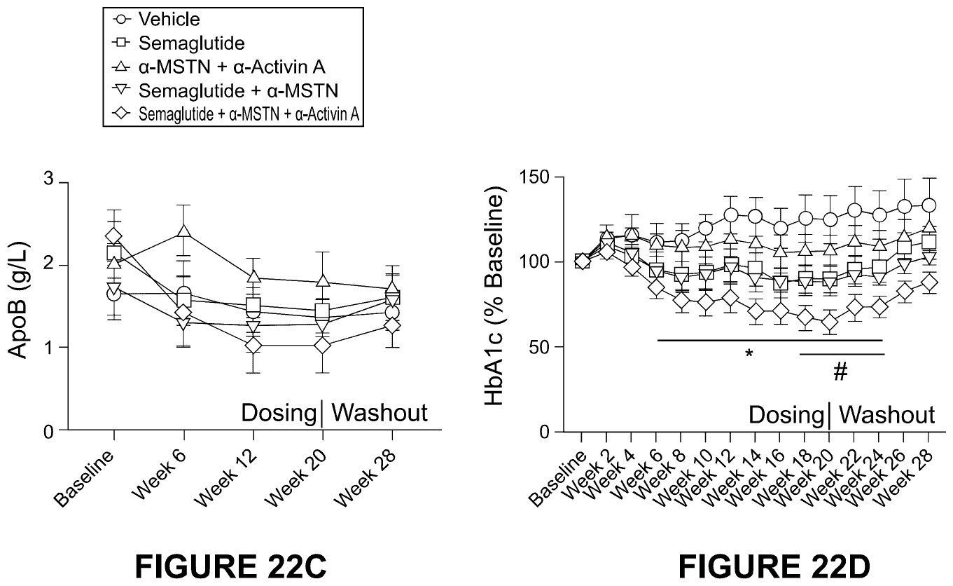

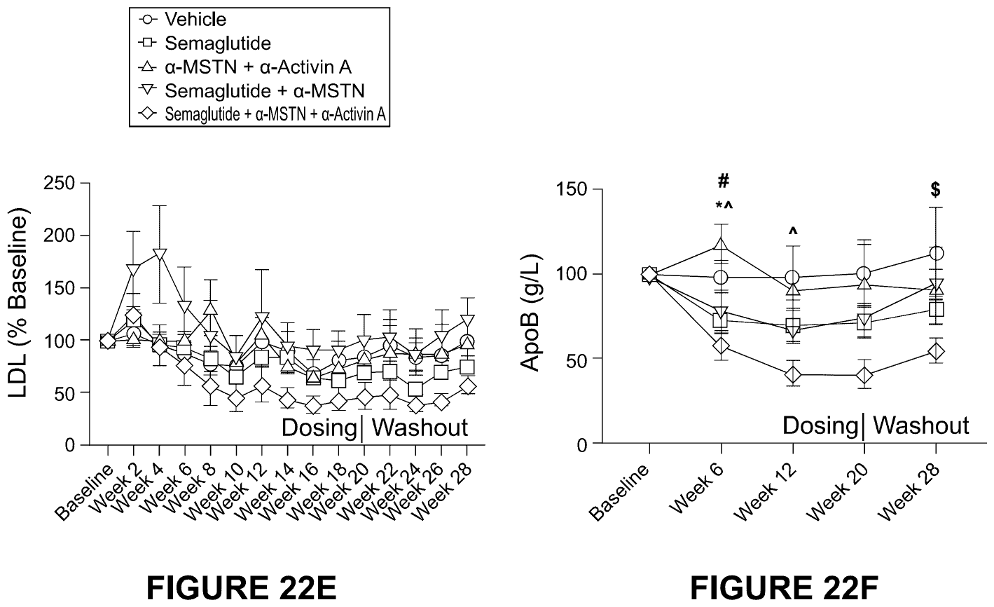

Activin A blockade on top of semaglutide and anti-myostatin antibody also induced further improvements in HbA1c and cholesterol. Measurements of HbA1c (FIG. 22A), LDL-C (FIG. 22B), ApoB (FIG. 22C), HbA1c change vs. baseline (FIG. 22D), LDL-C change vs. baseline (FIG. 22E), ApoB change vs. baseline (FIG. 22F) were made in the obese non-human primate treatment groups.

The combination treatment not only increased lean mass, but also improved HbA1c and cholesterol in the obese non-human primate subjects.

The present disclosure is not to be limited in scope by the specific embodiments described herein. Indeed, various modifications of the disclosure in addition to those described herein will become apparent to those skilled in the art from the foregoing description and the accompanying figures. Such modifications are intended to fall within the scope of the appended claims.

What is claimed is:

1. A composition comprising a Growth and Differentiation Factor-8 (GDF-8) inhibitor and a Glucagon-like peptide-1 (GLP-1) agonist.

2. A composition comprising a Growth and Differentiation Factor-8 (GDF-8) inhibitor, an Activin A inhibitor, and a Glucagon-like peptide-1 (GLP-1) agonist.

3. The composition of claim 1, wherein the GDF-8 inhibitor is a GDF8-specific binding protein.

4. The composition of claim 1, wherein the GDF-8 inhibitor is an antibody or antigen-binding fragment thereof that specifically binds GDF-8.

5. The composition of claim 4, wherein the anti-GDF8 antibody or antigen-binding fragment thereof comprises the heavy chain complementarity determining regions (HCDRs) of a heavy chain variable region (HCVR) comprising SEQ ID NO:4, and the light chain complementarity determining regions (LCDRs) of a light chain variable region (LCVR) comprising SEQ ID NO:5.

6. The composition of claim 4, wherein the anti-GDF8 antibody or antigen-binding fragment thereof comprises heavy chain complementarity determining regions (HCDR1, HCDR2, and HCDR3) comprising SEQ ID NO:6, SEQ ID NO:7, and SEQ ID NO:8, respectively, and three light chain complementarity determining regions (LCDR1, LCDR2, and LCDR3) comprising SEQ ID NO:9, TTS, and SEQ ID NO:11, respectively.

7. The composition of claim 2, wherein the Activin A inhibitor is an Activin A-specific binding protein.

8. The composition of claim 2, wherein the Activin A inhibitor is an antibody or antigen-binding fragment thereof that specifically binds Activin A.

9. The composition of claim 8, wherein the anti-Activin A antibody or antigen-binding fragment thereof comprises the heavy chain complementarity determining regions (HCDRs) of a heavy chain variable region (HCVR) comprising SEQ ID NO:12, and the light chain complementarity determining regions (LCDRs) of a light chain variable region (LCVR) comprising SEQ ID NO: 13.

10. The composition of claim 8, wherein the anti-Activin A antibody or antigen-binding fragment thereof comprises heavy chain complementarity determining regions (HCDR1, HCDR2, and HCDR3) comprising SEQ ID NO:14, SEQ ID NO: 15, and SEQ ID NO:16, respectively, and three light chain complementarity determining regions (LCDR1, LCDR2, and LCDR3) comprising SEQ ID NO:17, GAS, and SEQ ID NO:19, respectively.

11. The composition of claim 1, wherein the GLP-1 agonist is a GLP-1 receptor agonist.

12. The composition of claim 1, wherein the GLP-1 agonist is selected from the group consisting of Exenatide (long-acting), Dulaglutide, Liraglutide, Tirzepatide, and Semaglutide.

13. The composition of claim 12, wherein the GLP-1 agonist is Semaglutide.

14. The composition of claim 1, wherein the GLP-1 agonist is a GLP-1-specific binding protein.

15. The composition of claim 14, wherein the GLP-1 agonist is an antibody or antigen-binding fragment thereof that specifically binds GLP-1.

16. The composition of claim 1 for use in improving glucose control, increasing lean body mass, reducing fat mass, treating obesity, treating diabetes, and/or treating liver issues associated with increased fat mass, obesity, and/or diabetes in a subject.

17. A method for improving glucose control, increasing lean body mass, reducing fat mass, treating obesity, treating diabetes, and/or treating liver issues associated with increased fat mass, obesity, and/or diabetes in a subject, comprising administering a GDF8 inhibitor and a GLP-1 agonist to the subject.

18. A method for improving glucose control, increasing lean body mass, reducing fat mass, treating obesity, treating diabetes, and/or treating liver issues associated with increased fat mass, obesity, and/or diabetes in a subject, comprising administering a GDF8 inhibitor, an Activin A inhibitor, and a GLP-1 agonist to the subject.

19. The method of claim 17, wherein the GDF8 inhibitor and the GLP-1 agonist are administered to the subject in a single composition.

20. The method of claim 17, wherein the GDF8 inhibitor and the GLP-1 agonist are administered to the subject in at least two separate compositions.

21. The method of claim 17, wherein the GDF-8 inhibitor is a GDF8-specific binding protein.

22. The method of claim 17, wherein the GDF-8 inhibitor is an antibody or antigen-binding fragment thereof that specifically binds GDF-8.

23. The method of claim 22, wherein the anti-GDF8 antibody or antigen-binding fragment thereof comprises the heavy chain complementarity determining regions (HCDRs) of a heavy chain variable region (HCVR) comprising SEQ ID NO:4, and the light chain complementarity determining regions (LCDRs) of a light chain variable region (LCVR) comprising SEQ ID NO:5.

24. The method of claim 22, wherein the anti-GDF8 antibody or antigen-binding fragment thereof comprises heavy chain complementarity determining regions (HCDR1, HCDR2, and HCDR3) comprising SEQ ID NO:6, SEQ ID NO:7, and SEQ ID NO:8, respectively, and three light chain complementarity determining regions (LCDR1, LCDR2, and LCDR3) comprising SEQ ID NO:9, TTS, and SEQ ID NO:11, respectively.

25. The method of claim 18, wherein the Activin A inhibitor is an Activin A-specific binding protein.

26. The method of claim 18, wherein the Activin A inhibitor is an antibody or antigen-binding fragment thereof that specifically binds Activin A.

27. The method of claim 26, wherein the anti-Activin A antibody or antigen-binding fragment thereof comprises the heavy chain complementarity determining regions (HCDRs) of a heavy chain variable region (HCVR) comprising SEQ ID NO:12, and the light chain complementarity determining regions (LCDRs) of a light chain variable region (LCVR) comprising SEQ ID NO:13.

28. The method of claim 26, wherein the anti-Activin A antibody or antigen-binding fragment thereof comprises heavy chain complementarity determining regions (HCDR1, HCDR2, and HCDR3) comprising SEQ ID NO:14, SEQ ID NO:15, and SEQ ID NO:16, respectively, and three light chain complementarity determining regions (LCDR1, LCDR2, and LCDR3) comprising SEQ ID NO:17, GAS, and SEQ ID NO:19, respectively.

29. The method of claim 17, wherein the GLP-1 agonist is a GLP-1 receptor agonist.

30. The method of claim 17, wherein the GLP-1 agonist is selected from the group consisting of Exenatide (long-acting), Dulaglutide, Liraglutide, Tirzepatide, and Semaglutide.

31. The method of claim 30, wherein the GLP-1 agonist is Semaglutide.

32. The method of claim 17, wherein the GLP-1 agonist is a GLP-1-specific binding protein.

33. The method of claim 32, wherein the GLP-1 agonist is an antibody or antigen-binding fragment thereof that specifically binds GLP-1.

34. The method of claim 17, wherein the subject, at 12 weeks from administration of the inhibitor(s) and the agonist, exhibits at least one parameter change selected from the group consisting of:
i) at least about 35% decrease in fat mass;
ii) at least about 6% increase in lean mass;
iii) at least about 15% decrease in fasting glucose;
iv) at least about 6% decrease in HbA1c;
v) at least about 14% decrease in LDL;
vi) at least about 14% increase in LDL;
vii) at least about 35% decrease in NEFA; and
viii) at least about 55% decrease in TG.

35. The method of claim 34, wherein the subject, at 12 weeks from administration of the inhibitor(s) and the agonist, exhibits at least one parameter change selected from the group consisting of:
i) at least about 25% decrease in fasting glucose;
iv) at least about 25% decrease in HbA1c;
v) at least about 50% decrease in LDL;
vi) at least about 60% increase in LDL;
vii) at least about 50% decrease in NEFA; and
viii) at least about 65% decrease in TG.


SEQUENCE LISTING (28 sequences)

SEQ ID NO: 1 - protein, 109 aa, Homo sapiens
DFGLDCDEHSTESRCCRYPLTVDFEAFGWDWIIAPKRYKANYCSGECEFVFLQKYPHTHLVHQANPRGSAGPCCTPTKMSPINMLYFNGKEQIIYGKIPAMVVDRCGCS

SEQ ID NO: 2 - protein, 426 aa, Homo sapiens
MPLLWLRGFLLASCWIIVRSSPTPGSEGHSAAPDCPSCALAALPKDVPNSQPEMVEAVKKHILNMLHLKKRPDVTQPVPKAALLNAIRKLHVGKVGENGYVEIEDDIGRRAEMNELMEQTSEIITFAESGTARKTLHFEISKEGSDLSVVERAEVWLFLKVPKANRTRTKVTIRLFQQQKHPQGSLDTGEEAEEVGLKGERSELLLSEKVVDARKSTWHVFPVSSSIQRLLDQGKSSLDVRIACEQCQESGASLVLLGKKKKKEEEGEGKKKGGGEGGAGADEEKEQSHRPFLMLQARQSEDHPHRRRRRGLECDGKVNICCKKQFFVSFKDIGWNDWIIAPSGYHANYCEGECPSHIAGTSGSSLSFHSTVINHYRMRGHSPFANLKSCCVPTKLRPMSMLYYDDGQNIIKKDIQNMIVEECGCS

SEQ ID NO: 3 - protein, 411 aa, Homo sapiens
MDGLPGRALGAACLLLLAAGWLGPEAWGSPTPPPTPAAQPPPPPPGSPGGSQDTCTSCGGFRRPEELGRVDGDFLEAVKRHILSRLQMRGRPNITHAVPKAAMVTALRKLHAGKVREDGRVEIPHLDGHASPGADGQERVSEIISFAETDGLASSRVRLYFFISNEGNQNLFVVQASLWLYLKLLPYVLEKGSRRKVRVKVYFQEQGHGDRWNMVEKRVDLKRSGWHTFPLTEAIQALFERGERRLNLDVQCDSCQELAVVPVFVDPGEESHRPFVVVQARLGDSRHRIRKRGLECDGRTNLCCRQQFFIDFRLIGWNDWIIAPTSYYGNYCEGSCPAYLAGVPGSASSFHTAVVNQYRMRGLNPGTVNSCCIPTKLSTMSMLYFDDEYNIVKRDVPNMIVEECGCASPGK

SEQ ID NO: 4 - protein, 120 aa, synthetic construct
EVQVLESGGDLVQPGGSLRLSCAASGFTFSAYAMTWVRQAPGKGLEWVSAISGSGGSAYYADSVKGRFTISRDNSKNTVYLQMNSLRAEDTAVYYCAKDGAWKMSGLDVWGQGTTVIVSS

SEQ ID NO: 5 - protein, 107 aa, synthetic construct
DIQMTQSPASLSASVGDRVTITCRASQDISDYLAWYQQKPGKIPRLLIYTTSTLQSGVPSRFRGSGSGTDFTLTISSLQPEDVATYYCQKYDSAPLTFGGGTKVEIK

SEQ ID NO: 6 - protein, 8 aa, synthetic construct
GFTFSAYA

SEQ ID NO: 7 - protein, 8 aa, synthetic construct
ISGSGGSA

SEQ ID NO: 8 - protein, 13 aa, synthetic construct
AKDGAWKMSGLDV

SEQ ID NO: 9 - protein, 6 aa, synthetic construct
QDISDY

SEQ ID NO: 10 - skipped

SEQ ID NO: 11 - protein, 9 aa, synthetic construct
QKYDSAPLT

SEQ ID NO: 12 - protein, 126 aa, synthetic construct
QVQLQESGPGLVKPSETLSLTCTVSGGSFSSHFWSWIRQPPGKGLEWIGYILYTGGTSFNPSLKSRVSMSVGTSKNQFSLKLSSVTAADTAVYYCARARSGITFTGIIVPGSFDIWGQGTMVTVSS

SEQ ID NO: 13 - protein, 108 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPWTFGQGTKVEIK

SEQ ID NO: 14 - protein, 8 aa, synthetic construct
GGSFSSHF

SEQ ID NO: 15 - protein, 7 aa, synthetic construct
ILYTGGT

SEQ ID NO: 16 - protein, 20 aa, synthetic construct
ARARSGITFTGIIVPGSFDI

SEQ ID NO: 17 - protein, 7 aa, synthetic construct
QSVSSSY

SEQ ID NO: 18 - skipped

SEQ ID NO: 19 - protein, 9 aa, synthetic construct
QQYGSSPWT

SEQ ID NO: 20 - protein, 463 aa, Homo sapiens
MAGAPGPLRLALLLLGMVGRAGPRPQGATVSLWETVQKWREYRRQCQRSLTEDPPPATDLFCNRTFDEYACWPDGEPGSFVNVSCPWYLPWASSVPQGHVYRFCTAEGLWLQKDNSSLPWRDLSECEESKRGERSSPEEQLLFLYIIYTVGYALSFSALVIASAILLGFRHLHCTRNYIHLNLFASFILRALSVFIKDAALKWMYSTAAQQHQWDGLLSYQDSLSCRLVFLLMQYCVAANYYWLLVEGVYLYTLLAFSVLSEQWIFRLYVSIGWGVPLLFVVPWGIVKYLYEDEGCWTRNSNMNYWLIIRLPILFAIGVNFLIFVRVICIVVSKLKANLMCKTDIKCRLAKSTLTLIPLLGTHEVIFAFVMDEHARGTLRFIKLFTELSFTSFQGLMVAILYCFVNNEVQLEFRKSWERWRLEHLHIQRDSSMKPLKCPTSSLSSGATAGSSMYTATCQASCS

SEQ ID NO: 21 - protein, 125 aa, synthetic construct
EVQLVESGGGLVQPGRSLRLSCKASGFAFDDFAMHWVRQAPGKGLEWVSGIVWNSGDIGYADSVKGRFTISRDNAKNSLYLQLNSLRTEDTALYFCVKDMVRGLMGFNYYGMDVWGQGTTVTVSS

SEQ ID NO: 22 - protein, 107 aa, synthetic construct
EIVLTQSPATLSLSPGERATLSCRASQTISTYLVWYRQRPGQAPSLLIYDASNRATDIPARFSGSGSGTDFTLTISSLEPEDFAVYYCQQRSNWPITFGQGTRLEIK

SEQ ID NO: 23 - protein, 8 aa, synthetic construct
GFAFDDFA

SEQ ID NO: 24 - protein, 8 aa, synthetic construct
IVWNSGDI

SEQ ID NO: 25 - protein, 18 aa, synthetic construct
VKDMVRGLMGFNYYGMDV

SEQ ID NO: 26 - protein, 6 aa, synthetic construct
QTISTY

SEQ ID NO: 27 - skipped

SEQ ID NO: 28 - protein, 9 aa, synthetic construct
QQRSNWPIT